(19) United States
(12) Patent Application Publication
(10) Pub. No.: US 2019/0369114 A1
(43) Pub. Date: Dec. 5, 2019

(54) HIGHLY MULTIPLEXED AND MASS SPECTROMETRY BASED METHODS TO MEASURING 72 HUMAN PROTEINS

(71) Applicant: Cedars-Sinai Medical Center, Los Angeles, CA (US)

(72) Inventors: Jennifer VAN EYK, Los Angeles, CA (US); Qin FU, Beverly Hills, CA (US); Vidya VENKATRAMAN, Los Angeles, CA (US); Irina TCHERNYSHYOV, Baltimore, MD (US)

(73) Assignee: Cedars-Sinai Medical Center, Los Angeles, CA (US)

(21) Appl. No.: 16/515,458

(22) Filed: Jul. 18, 2019

Related U.S. Application Data
(63) Continuation of application No. PCT/US2018/014570, filed on Jan. 19, 2018.
(60) Provisional application No. 62/448,319, filed on Jan. 19, 2017.

(51) Int. Cl. G01N 33/68; C12Q 1/37

(52) U.S. Cl. CPC G01N 33/6848; C12Q 1/37; C12Y 304/21004; G01N 2560/00; G01N 2800/60; G01N 2800/50; G01N 2800/32

(57) ABSTRACT

Sample preparation for proteomic analysis of complex biological samples by mass spectrometry is a tedious and time-consuming process with many steps where technical variations can be introduced and propagated. We describe an automated trypsin digestion workflow that yields uniformly-processed samples in less than 5 hours. Reproducible quantitation of hundreds of peptides from numerous proteins was seen across replicates, days, instruments, and laboratory sites, demonstrating the broad applicability of this approach.

### CROSS-REFERENCE TO RELATED APPLICATION

This application claims the benefit under 35 U.S.C. § 119(e) of U.S. Provisional Patent Application No. 62/448,319 filed on Jan. 19, 2017, which is incorporated herein by reference in its entirety.

### STATEMENT REGARDING FEDERALLY SPONSORED RESEARCH OR DEVELOPMENT

This invention was made with government support under Grant No. HL111362 awarded by National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jan. 19, 2018, is named SequenceListing-065472-000652WO00_ST25.txt and is 791,213 bytes in size.

### FIELD OF THE INVENTION

Methods for obtaining a biomarker signature for a subject using mass spectrometry. A protein panel for determining the state of health of a subject, diagnosing disease in a subject, and determining risk of developing a disease in a subject.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The traditional biomarker development pipeline consists of two steps: shotgun proteomics for global protein discovery to create an in-depth protein inventory; and targeted proteomics for verification and validation aiming for high precision and high throughput protein quantitation. Liquid chromatography (LC) coupled with selected reaction monitoring mass spectrometry (SRM-MS) allows precise quantitation of proteotypic peptides (as surrogates for the corresponding protein) in 10s to 1000s of complex samples derived, for example, from bodily fluids, biopsies, or cultured cells, and is the method of choice for targeted biomarker validation. An emerging method, data-independent acquisition (DIA) mass spectrometry (MS), provides global and quantitative information simultaneously. Regardless of the MS approach, sample preparation, which mainly centers on enzymatic cleavage of proteins into a peptide mixture, is fundamental. However, throughput, reproducibility, time, and cost remain longstanding barriers to the necessary large-scale MS sample processing. The development of a fast, highly accurate, and completely hands-free MS protein sample preparation workflow would make the entire pathway from biomarker discovery to biomarker validation more robust.

Sample preparation for LC-MS/MS analysis is a multi-step process involving i) protein solubilization and denaturation, ii) disulfide bond reduction and cysteine-blocking to ensure consistent cysteine masses, iii) digestion of proteins into peptides with a site-specific protease (most often trypsin), and iv) clean-up to remove salts, denaturing agents, and other interfering compounds (typically by solid-phase extraction). Previous studies have shown that sample preparation, particularly the trypsin digestion step, is a major source for variability in the LC-MS/MS analysis. Optimal digestion conditions depend on both general and protein-specific factors including the trypsin-to-substrate ratio, buffer composition, protein structure, and the sequence and modifications (e.g., post translational modifications) adjacent to cleavage sites. It is, therefore, essential to have a highly controlled and standardized digestion method to meet the precision and reproducibility standards essential for reliable biomarker verification. To enhance precision and accuracy, each sample preparation step must have accurate liquid transfers, be initiated and stopped at a consistent time, be performed at a controlled temperature and have good mixing for uniform reactions. Automation provides a good solution for these requirements. In this communication, we describe an automated proteomics sample preparation workflow that yields reproducible quantitation data on complex proteomic samples and has been implemented in two different laboratories.

### SUMMARY OF THE INVENTION

The following embodiments and aspects thereof are described and illustrated in conjunction with systems, compositions and methods which are meant to be exemplary and illustrative, not limiting in scope.

Various embodiments of the present invention provide a method of obtaining a biomarker signature for a subject, the method comprising: obtaining a sample from the subject; treating the sample with a protease to obtain a digested sample; and measuring in the digested sample a representation of one or more biomarkers to obtain the biomarker signature for the subject, wherein the measuring is performed using a mass spectrometer and a mass spectrometry technique, and the representation of the one or more biomarkers is obtained using a mass spectrometry assay, wherein the mass spectrometry assay comprises a quantitation of two or more Q1/Q3 mass value pairs according to Table 5, wherein each Q1/Q3 mass value pair is used to quantify a corresponding peptide and protein according to Table 5. In some embodiments, the protease is trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof. In some embodiments, the protease is trypsin. In some embodiments, the mass spectrometer is a triple quadrupole mass spectrometer. In some embodiments, each Q1/Q3 mass value pair are correlated to the one or more peptides according to Table 5. In some embodiments, the one or more peptides are correlated to one or more proteins according to Table 5. In some embodiments, the one or more biomarkers are the one or more proteins according to Table 5. In some embodiments, the mass spectrometry technique is selected reaction monitoring (SRM) or multiple reaction monitoring (MRM). In some embodiments, the mass spectrometry technique is liquid chromatography-selected reaction monitoring-mass spectrometry (LC-SRM-MS). In some embodiments, the mass spectrometry technique is data-independent acquisition mass spectrometry (DIA MS). In some embodiments, the method further comprises adding a stable-isotope labeled peptide standard to the sample. In some embodiments, the method further comprises comparing the biomarker signature from the subject to a biomarker signature from a reference sample. In some embodiments, the method further comprises making an assessment of the subject based on the comparison, wherein the assessment is a diagnosis of a disease. In some embodiments, the sample is plasma, serum, cerebrospinal fluid (CSF), a tissue extract, or a biopsy sample. In some embodiments, the reference sample is obtained from a control subject, wherein the control subject does not have the disease. In some embodiments, the reference sample is obtained from the subject before the subject is treated for the disease. In some embodiments, the reference sample is from a subject that has been successfully treated for the disease.

Various embodiments of the present invention provide a method of treating a subject for a disease, the method comprising: (a) making an assessment of the subject based on a biomarker signature for the subject, wherein the assessment is a diagnosis of the disease; and (b) treating the subject based on the assessment, wherein the biomarker signature for the subject is obtained by a method comprising, obtaining a sample from the subject; treating the sample with a protease to obtain a digested sample; and measuring in the digested sample a representation of one or more biomarkers to obtain the biomarker signature for the subject, wherein the measuring is performed using a mass spectrometer and a mass spectrometry technique, and the representation of the one or more biomarkers is obtained using a mass spectrometry assay, wherein the mass spectrometry assay comprises a quantitation of two or more Q1/Q3 mass value pairs according to Table 5, wherein each Q1/Q3 mass value pair is used to quantify a corresponding peptide and protein according to Table 5. In some embodiments, the method further comprises adding a stable-isotope labeled peptide standard to the sample.

Various embodiments of the present invention provide a method of obtaining a biomarker signature for a subject, the method comprising: obtaining a sample from the subject; treating the sample with a protease to obtain a digested sample; and measuring in the digested sample a representation of one or more biomarkers to obtain the biomarker signature for the subject, wherein the measuring is performed using a mass spectrometer and a mass spectrometry technique, and the representation of the one or more biomarkers is obtained using a mass spectrometry assay, wherein the mass spectrometry assay comprises a quantitation of two or more Q1 mass values according to Table 5, wherein each Q1 mass value is used to quantify a corresponding peptide and protein according to Table 5. In some embodiments, the protease is trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof. In some embodiments, the protease is trypsin. In some the mass spectrometer is a quadrupole time-of-flight (QTOF) mass spectrometer or a hybrid quadrupole-orbitrap (QOrbitrap) mass spectrometer. In some embodiments, each Q1 mass value is correlated to the one or more peptides according to Table 5. In some embodiments, the one or more peptides are correlated to one or more proteins according to Table 5. In some embodiments, the one or more biomarkers are the one or more proteins according to Table 5. In some embodiments, the mass spectrometry technique is parallel reaction monitoring (PRM). In some embodiments, the mass spectrometry technique is liquid chromatography-parallel reaction monitoring-mass spectrometry (LC-PRM-MS). In some embodiments, the method further comprises adding a stable-isotope labeled peptide standard to the sample. In some embodiments, the method further comprises comparing the biomarker signature from the subject to a biomarker signature from a reference sample. In some embodiments, the method further comprises making an assessment of the subject based on the comparison, wherein the assessment is a diagnosis of a disease. In some embodiments, the sample is plasma, serum, cerebrospinal fluid (CSF), a tissue extract, or a biopsy sample. In some embodiments, the reference sample is obtained from a control subject, wherein the control subject does not have the disease. In some embodiments, the reference sample is obtained from the subject before the subject is treated for the disease. In some embodiments, the reference sample is from a subject that has been successfully treated for the disease.

Various embodiments of the present invention provide a method of treating a subject for a disease, the method comprising: (a) making an assessment of the subject based on a biomarker signature for the subject, wherein the assessment is a diagnosis of the disease; and (b) treating the subject based on the assessment, wherein the biomarker signature for the subject is obtained by a method comprising: obtaining a sample from the subject; treating the sample with a protease to obtain a digested sample; and measuring in the digested sample a representation of one or more biomarkers to obtain the biomarker signature for the subject, wherein the measuring is performed using a mass spectrometer and a mass spectrometry technique, and the representation of the one or more biomarkers is obtained using a mass spectrometry assay, wherein the mass spectrometry assay comprises a quantitation of two or more Q1 mass values according to Table 5, wherein each Q1 mass value is used to quantify a corresponding peptide and protein according to Table 5. In some embodiments, the method further comprises adding a stable-isotope labeled peptide standard to the sample.

Various embodiments of the present invention provide a method for assessing the efficacy of a treatment, comprising: comparing a biomarker signature from a subject to a biomarker signature from a reference sample, wherein a change in the biomarker signature from the subject relative to the biomarker signature from the reference sample is indicative of the efficacy of the treatment, wherein the treatment is according to a method of treating a subject for a disease, the method comprising: (a) making an assessment of the subject based on the biomarker signature, wherein the assessment is a diagnosis of the disease; and (b) treating the subject based on the assessment, wherein the biomarker signature for the subject is obtained by a method comprising, obtaining a sample from the subject; treating the sample with a protease to obtain a digested sample; and measuring in the digested sample a representation of one or more biomarkers to obtain the biomarker signature for the subject, wherein the measuring is performed using a mass spectrometer and a mass spectrometry technique, and the representation of the one or more biomarkers is obtained using a mass spectrometry assay, wherein the mass spectrometry assay comprises a quantitation of two or more Q1/Q3 mass value pairs according to Table 5, wherein each Q1/Q3 mass value pair is used to quantify a corresponding peptide and protein according to Table 5. In some embodiments, the reference sample is obtained from a control subject, wherein the control subject does not have the disease. In some embodiments, the reference sample is obtained from the subject before the subject is treated for the disease. In some embodiments, the reference sample is from a subject that has been successfully treated for the disease. In some embodiments, the method further comprises adding a stable-isotope labeled peptide standard to the sample.

Various embodiments of the present invention provide a method for assessing the efficacy of a treatment, comprising: comparing a biomarker signature from a subject to a biomarker signature from a reference sample, wherein a change in the biomarker signature from the subject relative to the biomarker signature from the reference sample is indicative of the efficacy of the treatment, wherein the treatment is according to a method of treating a subject for a disease, the method comprising: (a) making an assessment of the subject based on the biomarker signature, wherein the assessment is a diagnosis of the disease; and (b) treating the subject based on the assessment, wherein the biomarker signature for the subject is obtained by a method comprising: obtaining a sample from the subject; treating the sample with a protease to obtain a digested sample; and measuring in the digested sample a representation of one or more biomarkers to obtain the biomarker signature for the subject, wherein the measuring is performed using a mass spectrometer and a mass spectrometry technique, and the representation of the one or more biomarkers is obtained using a mass spectrometry assay, wherein the mass spectrometry assay comprises a quantitation of two or more Q1 mass values according to Table 5, wherein each Q1 mass value is used to quantify a corresponding peptide and protein according to Table 5. In some embodiments, the reference sample is obtained from a control subject, wherein the control subject does not have the disease. In some embodiments, the reference sample is obtained from the subject before the subject is treated for the disease. In some embodiments, the reference sample is from a subject that has been successfully treated for the disease. In some embodiments, the method further comprises adding a stable-isotope labeled peptide standard to the sample.

In various embodiments, the present invention provides a protein panel for assessing and/or determining the state of health of a subject, the protein panel comprising one or more proteins listed in Table 15. In some embodiments, the one or more proteins are biomarkers for one or more functions or combination thereof. In some embodiments, the functions are inflammatory response, lipid metabolism, redox signaling, immune response, or endothelial dysfunction. In some embodiments, the functions are lipid metabolism, immune response, coagulation, inflammatory response, or redox signaling. In some embodiments, the function is inflammatory response and the proteins are any one or more of Alpha-2-macroglobulin (UniProt Accession No. P01023) (SEQ ID NO: 15), Angiotensinogen (UniProt Accession No. P01019) (SEQ ID NO: 14), Alpha-2-HS-glycoprotein (UniProt Accession No. P02765) (SEQ ID NO: 35), Apolipoprotein A-I (UniProt Accession No. P02647) (SEQ ID NO: 21), Apolipoprotein E (UniProt Accession No. P02649) (SEQ ID NO: 22), Complement C3 (UniProt Accession No. P01024) (SEQ ID NO: 16), Complement C5 (UniProt Accession No. P01031) (SEQ ID NO: 17), Complement factor H (UniProt Accession No. P08603) (SEQ ID NO: 56), C-reactive protein (UniProt Accession No. P02741) (SEQ ID NO: 28), Coagulation factor XII (UniProt Accession No. P00748) (SEQ ID NO: 9), Prothrombin (UniProt Accession No. P00734) (SEQ ID NO: 4), Vitamin D-binding protein (UniProt Accession No. P02774) (SEQ ID NO: 37), Gelsolin (UniProt Accession No. P06396) (SEQ ID NO: 51), Haptoglobin (UniProt Accession No. P00738) (SEQ ID NO: 6), Ig mu chain C region (UniProt Accession No. P01871) (SEQ ID NO: 19), Plasma kallikrein (UniProt Accession No. P03952) (SEQ ID NO: 40), Apolipoprotein(a) (UniProt Accession No. P08519) (SEQ ID NO: 55), Lumican (UniProt Accession No. P51884) (SEQ ID NO: 69), Alpha-1-acid glycoprotein 1 (UniProt Accession No. P02763) (SEQ ID NO: 34), Plasminogen (UniProt Accession No. P00747) (SEQ ID NO: 8), Serum paraoxonase/arylesterase 1 (UniProt Accession No. P27169) (SEQ ID NO: 65), Alpha-1-antitrypsin (UniProt Accession No. P01009) (SEQ ID NO: 12), Alpha-1-antichymotrypsin (UniProt Accession No. P01011) (SEQ ID NO: 13), Plasma protease C1 inhibitor (UniProt Accession No. P05155) (SEQ ID NO: 48), Vitronectin (UniProt Accession No. P04004) (SEQ ID NO: 42) or combinations thereof. In some embodiments, the function is lipid metabolism and the proteins are any one or more of Angiotensinogen (UniProt Accession No. P01019) (SEQ ID NO: 14), Alpha-2-HS-glycoprotein (UnitProt Accession No. P02765) (SEQ ID NO: 35), Serum albumin (UniProt Accession No. P02768) (SEQ ID NO: 36), Apolipoprotein A-I (UniProt Accession No. P02647) (SEQ ID NO: 21), Apolipoprotein A-II (UniProt Accession No. P02652) (SEQ ID NO: 23), Apolipoprotein A-IV (UniProt Accession No. P06727) (SEQ ID NO: 52), Apolipoprotein B-100 (UniProt Accession No. P04114) (SEQ ID NO: 43), Apolipoprotein C-I (UniProt Accession No. P02654) (SEQ ID NO: 24), Apolipoprotein C-III (UniProt Accession No. P02656) (SEQ ID NO: 26), Apolipoprotein E (UniProt Accession No. P02649) (SEQ ID NO: 22), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Complement factor H (UniProt Accession No. P08603) (SEQ ID NO: 56), Clusterin (UniProt Accession No. P10909) (SEQ ID NO: 61), C-reactive protein (UniProt Accession No. P02741) (SEQ ID NO: 28), Prothrombin (UniProt Accession No. P00734) (SEQ ID NO: 4), Vitamin D-binding protein (UniProt Accession No. P02774) (SEQ ID NO: 37), Haptoglobin (UniProt Accession No. P00738) (SEQ ID NO. 6), Plasma kallikrein (UniProt Accession No. P03952) (SEQ ID NO: 40), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), sex hormone binding globulin (UniProt Accession No. P04278) (SEQ ID NO: 46) or combinations thereof. In some embodiments, the function is redox signaling and the proteins are any one or more of Angiotensinogen (UniProt Accession No. P01019) (SEQ ID NO: 14), Serum albumin (UniProt Accession No. P02768) (SEQ ID NO: 36), Apolipoprotein A-I (UniProt Accession No. P02647) (SEQ ID NO: 21), Apolipoprotein A-IV (UniProt Accession No. P06727) (SEQ ID NO: 52), Apolipoprotein C-II (UniProt Accession No. P02655) (SEQ ID NO: 25), Apolipoprotein E (UniProt Accession No. P02649) (SEQ ID NO: 22), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Complement factor H (UniProt Accession No. P08603) (SEQ ID NO: 56), C-reactive protein (UniProt Accession No. P02741) (SEQ ID NO: 28), Prothrombin (UniProt Accession No. P00734) (SEQ ID NO: 4), Gelsolin (UniProt Accession No. P06396) (SEQ ID NO: 51), Hemoglobin subunit alpha (UniProt Accession No. P69905) (SEQ ID NO: 70), Haptoglobin (UniProt Accession No. P00738) (SEQ ID NO: 6), Inter-alpha-trypsin inhibitor heavy chain H4 (UniProt Accession No. Q14624) (SEQ ID NO: 71), Serum paraoxonase/lactonase 3 (UniProt Accession No. Q15166) (SEQ ID NO: 72), Peroxiredoxin-2 (UniProt Accession No. P32119) (SEQ ID NO: 66), Alpha-1-antitrypsin (UniProt Accession No. P01009) (SEQ ID NO: 12), Alpha-1-antichymotrypsin (UniProt Accession No. P01011) (SEQ ID NO: 13), Serotransferrin (UniProt Accession No. P02787) (SEQ ID NO: 38), or combinations thereof. In some embodiments, the function is immune response and the proteins are any one or more of Complement C1q subcomponent subunit B (UniProt Accession No. P02746) (SEQ ID NO: 29), Complement C1q subcomponent subunit C (UniProt Accession No. P02747) (SEQ ID NO: 30), Complement C1r subcomponent (UniProt Accession No. P00736) (SEQ ID NO: 50), Complement C1s subcomponent (UniProt Accession No. P09871) (SEQ ID NO: 58), Complement C3 (UniProt Accession No. P01024) (SEQ ID NO: 16), Complement C4-A (UniProt Accession No. P0C0L4) (SEQ ID NO: 59), Complement C5 (UniProt Accession No. P01031) (SEQ ID NO: 17), Complement component C8 alpha chain (UniProt Accession No. P07357) (SEQ ID NO: 53), Complement component C9 (UniProt Accession No. P02748) (SEQ ID NO: 31), Complement factor B (UniProt Accession No. P00751) (SEQ ID NO: 10), Plasma protease C1 inhibitor (UniProt Accession No. P05155) (SEQ ID NO: 48), or combinations thereof. In some embodiments, the function is endothelial dysfunction and the proteins are any one or more of Angiotensinogen (UniProt Accession No. P01019) (SEQ ID NO: 14), C-reactive protein (UniProt Accession No. P02741) (SEQ ID NO: 28), Lumican (UniProt Accession No. P51884) (SEQ ID NO: 69), Plasminogen (UniProt Accession No. P00747) (SEQ ID NO: 8), Peroxiredoxin-2 (UniProt Accession No. P32119) (SEQ ID NO: 66), Alpha-1-antitrypsin (UniProt Accession No. P01009) (SEQ ID NO: 12), Alpha-1-anti chymotrypsin (UniProt Accession No. P01011) (SEQ ID NO: 13), Antithrombin-III (UniProt Accession No. P01008) (SEQ ID NO: 11), or combinations thereof. In some embodiments, the function is coagulation and the proteins are any one or more of Serum albumin (UniProt Accession No. P02768) (SEQ ID NO: 36), Apolipoprotein E (UniProt Accession No. P02649) (SEQ ID NO: 22), Complement C3 (UniProt Accession No. P01024) (SEQ ID NO: 16), Complement C4-A (UniProt Accession No. P0C0L4) (SEQ ID NO: 59), Complement C4-B (UniProt Accession No. P0C0L5) (SEQ ID NO: 60), Complement C5 (UniProt Accession No. P01031) (SEQ ID NO: 17), Complement component C9 (UniProt Accession No. P02748) (SEQ ID NO: 31), Complement factor B (UniProt Accession No. P00751) (SEQ ID NO: 10), Complement factor H (UniProt Accession No.) (SEQ ID NO: 56), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Fibrinogen (UniProt Accession No. P02671) (SEQ ID NO: 27), Haptoglobin (UniProt Accession No. P00738) (SEQ ID NO: 6), Integrin alpha-Iib (UniProt Accession No. P08514) (SEQ ID NO: 54), Plasma kallikrein (UniProt Accession No. P03952) (SEQ ID NO: 40), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Peroxiredoxin-2 (UniProt Accession No. P32119) (SEQ ID NO: 66), Plasma serine protease inhibitor (UniProt Accession No. P05154) (SEQ ID NO: 47), Antithrombin-III (UniProt Accession No. P01008) (SEQ ID NO: 11), Heparin cofactor 2 (UniProt Accession No. P05546) (SEQ ID NO: 50), Alpha-2-antiplasmin (UniProt Accession No. P08697) (SEQ ID NO: 57), Vitronectin (UniProt Accession No. P04004) (SEQ ID NO: 42), von Willebrand factor (UniProt Accession No. P04275) (SEQ ID NO: 45), or combinations thereof. In some embodiments, the function is lipid metabolism and the proteins are any one or more of Angiotensinogen (UniProt Accession No. P01019) (SEQ ID NO: 14), Alpha-2-HS-glycoprotein (UnitProt Accession No. P02765) (SEQ ID NO: 35), Serum albumin (UniProt Accession No. P02768) (SEQ ID NO: 36), Apolipoprotein A-I (UniProt Accession No. P02647) (SEQ ID NO: 21), Apolipoprotein A-II (UniProt Accession No. P02652) (SEQ ID NO: 23), Apolipoprotein A-IV (UniProt Accession No. P06727) (SEQ ID NO: 52), Apolipoprotein B-100 (UniProt Accession No. P04114) (SEQ ID NO: 43), Apolipoprotein C-I (UniProt Accession No. P02654) (SEQ ID NO: 24), Apolipoprotein C-III (UniProt Accession No. P02656) (SEQ ID NO: 26), Apolipoprotein E (UniProt Accession No. P02649) (SEQ ID NO: 22), Zinc-alpha-2-glycoprotein (UniProt Accession No. P25311) (SEQ ID NO: 64), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Complement factor H (UniProt Accession No. P08603) (SEQ ID NO: 56), Clusterin (UniProt Accession No. P10909) (SEQ ID NO: 61), C-reactive protein (UniProt Accession No. P02741) (SEQ ID NO: 28), Prothrombin (UniProt Accession No. P00734) (SEQ ID NO: 4), Vitamin D-binding protein (UniProt Accession No. P02774) (SEQ ID NO: 37), Haptoglobin (UniProt Accession No. P00738) (SEQ ID NO. 6), Plasma kallikrein (UniProt Accession No. P03952) (SEQ ID NO: 40), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), Alpha-1-antitrypsin (UniProt Accession No. P01009) (SEQ ID NO: 12), sex hormone binding globulin (UniProt Accession No. P04278) (SEQ ID NO: 46) or combinations thereof. In some embodiments, the function is inflammatory response and the proteins are any one or more of Alpha-2-macroglobulin (UniProt Accession No. P01023) (SEQ ID NO: 15), Angiotensinogen (UniProt Accession No. P01019) (SEQ ID NO: 14), Alpha-2-HS-glycoprotein (UniProt Accession No. P02765) (SEQ ID NO: 35), Apolipoprotein A-I (UniProt Accession No. P02647) (SEQ ID NO: 21), Apolipoprotein E (UniProt Accession No. P02649) (SEQ ID NO: 22), Complement C3 (UniProt Accession No. P01024) (SEQ ID NO: 16), Complement C5 (UniProt Accession No. P01031) (SEQ ID NO: 17), Complement factor H (UniProt Accession No. P08603) (SEQ ID NO: 56), C-reactive protein (UniProt Accession No. P02741) (SEQ ID NO: 28), C-reactive protein (UniProt Accession No. P02741) (SEQ ID NO: 28), Coagulation factor XII (UniProt Accession No. P00748) (SEQ ID NO: 9), Prothrombin (UniProt Accession No. P00734) (SEQ ID NO: 4), Vitamin D-binding protein (UniProt Accession No. P02774) (SEQ ID NO: 37), Gelsolin (UniProt Accession No. P06396) (SEQ ID NO: 51), Haptoglobin (UniProt Accession No. P00738) (SEQ ID NO: 6), Ig mu chain C region (UniProt Accession No. P01871) (SEQ ID NO: 19), Plasma kallikrein (UniProt Accession No. P03952) (SEQ ID NO: 40), Apolipoprotein(a) (UniProt Accession No. P08519) (SEQ ID NO: 55), Lumican (UniProt Accession No. P51884) (SEQ ID NO: 69), Alpha-1-acid glycoprotein 1 (UniProt Accession No. P02763) (SEQ ID NO: 34), Plasminogen (UniProt Accession No. P00747) (SEQ ID NO: 8), Serum paraoxonase/arylesterase 1 (UniProt Accession No. P27169) (SEQ ID NO: 65), Alpha-1-antitrypsin (UniProt Accession No. P01009) (SEQ ID NO: 12), Alpha-1-antichymotrypsin (UniProt Accession No. P01011) (SEQ ID NO: 13), Plasma protease C1 inhibitor (UniProt Accession No. P05155) (SEQ ID NO: 48), Vitronectin (UniProt Accession No. P04004) (SEQ ID NO: 42) or combinations thereof. In some embodiments, the function is immune response and the proteins are any one or more of Angiotensinogen (UniProt Accession No. P01019) (SEQ ID NO: 14), Alpha-2-HS-glycoprotein (UniProt Accession No. P02765) (SEQ ID NO: 35), Apolipoprotein A-I (UniProt Accession No. P02647) (SEQ ID NO: 21), Apolipoprotein A-II (UniProt Accession No. P02652) (SEQ ID NO: 23), Apolipoprotein E (UniProt Accession No. P02649) (SEQ ID NO: 22), Complement C1q subcomponent subunit B (UniProt Accession No. P02746) (SEQ ID NO: 29), Complement C1q subcomponent subunit C (UniProt Accession No. P02747) (SEQ ID NO: 30), Complement C1r subcomponent (UniProt Accession No. P00736) (SEQ ID NO: 50), Complement C1s subcomponent (UniProt Accession No. P09871) (SEQ ID NO: 58), Complement C3 (UniProt Accession No. P01024) (SEQ ID NO: 16), Complement C4-A (UniProt Accession No. P0C0L4) (SEQ ID NO: 59), Complement C4-B (UniProt Accession No. P0C0L5) (SEQ ID NO: 60), Complement C5 (UniProt Accession No. P01031) (SEQ ID NO: 17), Complement component C8 alpha chain (UniProt Accession No. P07357) (SEQ ID NO: 53), Complement component C9 (UniProt Accession No. P02748) (SEQ ID NO: 31), Complement factor B (UniProt Accession No. P00751) (SEQ ID NO: 10), Complement factor H (UniProt Accession No. P08603) (SEQ ID NO: 56), C-reactive protein (UniProt Accession No. P02741) (SEQ ID NO: 28), Coagulation factor X (UniProt Accession No. P00742) (SEQ ID NO: 7), Gelsolin (UniProt Accession No. P06396) (SEQ ID NO: 51), Ig alpha-1 chain C region (UniProt Accession No. P01876) (SEQ ID NO: 20), Ig gamma-3 chain C region (UniProt Accession No. P01860) (SEQ ID NO: 18), Ig mu chain C region (UniProt Accession No. P01871) (SEQ ID NO: 19), Lumican (UniProt Accession No. P51884) (SEQ ID NO: 69), Serum paraoxonase/arylesterase 1 (UniProt Accession No. P27169) (SEQ ID NO: 65), Plasma protease C1 inhibitor (UniProt Accession No. P05155) (SEQ ID NO: 48), Vitronectin (UniProt Accession No. P04004) (SEQ ID NO:), von Willebrand factor (UniProt Accession No. P04275) (SEQ ID NO: 45), or combinations thereof. In some embodiments, the one or more proteins are biomarkers for one or more diseases or combination thereof. In some embodiments, the disease is cardiovascular disease. In some embodiments, the disease is cardiovascular disease and the proteins are any one or more of Angiotensinogen (UniProt Accession No. P01019) (SEQ ID NO: 14), Apolipoprotein A-I (UniProt Accession No. P02647) (SEQ ID NO: 21), Apolipoprotein B-100 (UniProt Accession No. P04114) (SEQ ID NO: 43), Apolipoprotein E (UniProt Accession No. P02649) (SEQ ID NO: 22), Complement C3 (UniProt Accession No. P01024) (SEQ ID NO: 16), Complement C4-A (UniProt Accession No. P0C0L4) (SEQ ID NO: 59), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Gelsolin (UniProt Accession No. P06396) (SEQ ID NO: 51), Integrin alpha-Iib (UniProt Accession No. P08514) (SEQ ID NO: 54), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Antithrombin-III (UniProt Accession No. P01008) (SEQ ID NO: 11), Plasma protease C1 inhibitor (UniProt Accession No. P05155) (SEQ ID NO: 48), von Willebrand factor (UniProt Accession No. P04275) (SEQ ID NO: 45), or combinations thereof. In some embodiments, the disease is cardiovascular disease and the proteins are any one or more of Angiotensinogen (UniProt Accession No. P01019) (SEQ ID NO: 14), Apolipoprotein A-I (UniProt Accession No. P02647) ((SEQ ID NO: 21), Apolipoprotein B-100 (UniProt Accession No. P04114) (SEQ ID NO: 43), Apolipoprotein E (UniProt Accession No. P02649) (SEQ ID NO: 22), Complement C3 (UniProt Accession No. P01024) (SEQ ID NO: 16), Complement C4-A (UniProt Accession No. P0C0L4) (SEQ ID NO: 59), Complement C4-B (UniProt Accession No. P0C0L5) (SEQ ID NO: 60), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Gelsolin (UniProt Accession No. P06396) (SEQ ID NO: 51), Integrin alpha-Iib (UniProt Accession No. P08514) (SEQ ID NO: 54), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Antithrombin-III (UniProt Accession No. P01008) (SEQ ID NO: 11), Alpha-2-antiplasmin (UniProt Accession No. P08697) (SEQ ID NO: 57), Plasma protease C1 inhibitor (UniProt Accession No. P05155) (SEQ ID NO: 48), von Willebrand factor (UniProt Accession No. P04275) (SEQ ID NO: 45), or combinations thereof. In some embodiments, the cardiovascular disease is selected from congestive heart failure, arrhythmia, pericarditis, acute myocardial infarction, infarcted myocardium, coronary artery disease, coronary heart disease, ischemic heart disease, cardiomyopathy, stroke, hypertensive heart disease, heart failure, pulmonary heart disease, ischemic syndrome, coronary microvascular disease, cardiac dysrhythmias, rheumatic heart disease, aortic aneurysms, cardiomyopathy, atrial fibrillation, congenital heart disease, endocarditis, inflammatory heart disease, inflammatory cardiomegaly, myocarditis, valvular heart disease, cerebrovascular disease, peripheral artery disease or any combination thereof. In some embodiments, the disease is atherosclerosis and the proteins are any one or more of Angiotensinogen (UniProt Accession No. P01019) (SEQ ID NO: 14), Alpha-2-HS-glycoprotein (UniProt Accession No. P02765) (SEQ ID NO: 35), Apolipoprotein A-I (UniProt Accession No. P02647) (SEQ ID NO: 21), Apolipoprotein A-II (Uniprot Accession No. P02652) (SEQ ID NO: 23), Apolipoprotein A-IV (UniProt Accession No. P06727) (SEQ ID NO: 52), Apolipoprotein B-100 (UniProt Accession No. P04114) (SEQ ID NO: 43), Apolipoprotein C-I (UniProt Accession No. P02654) (SEQ ID NO: 24), Apolipoprotein C-II (UniProt Accession No. P02655) (SEQ ID NO: 25), Apolipoprotein (UniProt Accession No. P02656) (SEQ ID NO. 26), Apolipoprotein E (UniProt Accession No. P02649) (SEQ ID NO: 22), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Prothrombin (UniProt Accession No. P00734) (SEQ ID NO: 4), Hemoglobin subunit alpha (UniProt Accession No. P69905) (SEQ ID NO: 70), Haptoglobin (UniProt Accession No. P00738) (SEQ ID NO: 6), Integrin alpha-Iib (UniProt Accession No. P08514) (SEQ ID NO: 54), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), Peroxiredoxin-2 (UniProt Accession No. P32119) (SEQ ID NO: 66), Antithrombin-III (UniProt Accession No. P01008) (SEQ ID NO. 11), Heparin cofactor 2 (UniProt Accession No. P05546) (SEQ ID NO: 50), von Willebrand factor (UniProt Accession No. P04275) (SEQ ID NO: 45) or combinations thereof. In some embodiments, the disease is renal failure and the proteins are any one or more of Angiotensinogen (UniProt Accession No. P01019) (SEQ ID NO: 14), Serum albumin (UniProt Accession No. P02768) (SEQ ID NO: 36), Apolipoprotein B-100 (UniProt Accession No. P04114) (SEQ ID NO: 43), Complement C3 (UniProt Accession No. P01024) (SEQ ID NO: 16), Complement C4-A (UniProt Accession No. P0C0L4) (SEQ ID NO: 59), Complement C5 (UniProt Accession No. P01031) (SEQ ID NO: 17), Complement factor B (UniProt Accession No. P00751) (SEQ ID NO: 10), C-reactive protein (UniProt Accession No. P02741) (SEQ ID NO: 28), Coagulation factor X (UniProt Accession No. P00742) (SEQ ID NO: 7), Prothrombin (UniProt Accession No. P00734) (SEQ ID NO: 4), Hemoglobin subunit alpha (UniProt Accession No. P69905) (SEQ ID NO: 70), Antithrombin-III (UniProt Accession No. P01008) (SEQ ID NO: 11), Serotransferrin (UniProt Accession No. P02787) (SEQ ID NO: 38), or combinations thereof. In some embodiments, the disease is renal failure and the proteins are any one or more of Angiotensinogen (UniProt Accession No. P01019) (SEQ ID NO: 14), Serum albumin (UniProt Accession No. P02768) (SEQ ID NO: 36), Apolipoprotein B-100 (UniProt Accession No. P04114) (SEQ ID NO: 43), Complement C3 (UniProt Accession No. P01024) (SEQ ID NO: 16), Complement C4-A (UniProt Accession No. P0C0L4) (SEQ ID NO: 59), Complement C5 (UniProt Accession No. P01031) (SEQ ID NO: 17), Complement factor B (UniProt Accession No. P00751) (SEQ ID NO: 10), C-reactive protein (UniProt Accession No. P02741) (SEQ ID NO: 28), Coagulation factor X (UniProt Accession No. P00742) (SEQ ID NO: 7), Prothrombin (UniProt Accession No. P00734) (SEQ ID NO: 4), Hemoglobin subunit alpha (UniProt Accession No. P69905) (SEQ ID NO: 70), Antithrombin-III (UniProt Accession No. P01008) (SEQ ID NO: 11), Serotransferrin (UniProt Accession No. P02787) (SEQ ID NO: 38), or combinations thereof. In some embodiments, the disease is liver disease and the proteins are any one or more of Alpha-1B-glycoprotein (UniProt Accession No. P04217) (SEQ ID NO: 44), Alpha-2-macroglobulin (UniProt Accession No. P01023) (SEQ ID NO: 15), Afamin (UniProt Accession No. P43652) (SEQ ID NO: 68), Angiotensinogen (UniProt Accession No. P01019) (SEQ ID NO: 14), Alpha-2-HS-glycoprotein (UniProt Accession No. P02765) (SEQ ID NO: 35), Serum albumin (UniProt Accession No. P02768) (SEQ ID NO: 36), Protein AMBP (UniProt Accession No. P02760) (SEQ ID NO: 33), Apolipoprotein A-I (UniProt Accession No. P02647) (SEQ ID NO: 21), Apolipoprotein A-II (UniProt Accession No. P02652) (SEQ ID NO: 23), Apolipoprotein B-100 (UniProt Accession No. P04114) (SEQ ID NO: 43), Apolipoprotein C-I (UniProt Accession No. P02654) (SEQ ID NO: 24), Apolipoprotein E (UniProt Accession No. P02649) (SEQ ID NO: 22), Apolipoprotein L1 (UniProt Accession No. 014791) (SEQ ID NO: 1), Complement C1q subcomponent subunit C (UniProt Accession No. P02747) (SEQ ID NO: 30), Complement C1r subcomponent (UniProt Accession No. P00736) (SEQ ID NO: 5), Complement C1s subcomponent (UniProt Accession No. P09871) (SEQ ID NO: 58), Complement C3 (UniProt Accession No. P01024) (SEQ ID NO: 16), Complement C4-A (UniProt Accession No. P0C0L4) (SEQ ID NO: 59), Complement C4-B (UniProt Accession No. P0C0L5) (SEQ ID NO: 60), C4b-binding protein alpha chain (UniProt Accession No. P04003) (SEQ ID NO: 41), Complement C5 (UniProt Accession No. P01031) (SEQ ID NO: 17), Complement component C8 alpha chain (UniProt Accession No. P07357) (SEQ ID NO: 53), Complement component C9 (UniProt Accession No. P02748) (SEQ ID NO: 31), Complement factor B (UniProt Accession No. P00751) (SEQ ID NO: 10), Complement factor H (UniProt Accession No. P08603) (SEQ ID NO: 56), Clusterin (UniProt Accession No. P10909) (SEQ ID NO: 61), Ceruloplasmin (UniProt Accession No. P00450) (SEQ ID NO: 2), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Fibrinogen (UniProt Accession No. P02671) (SEQ ID NO: 27), Vitamin D-binding protein (UniProt Accession No. P02774) (SEQ ID NO: 37), Gelsolin (UniProt Accession No. P06396) (SEQ ID NO: 51), Hemoglobin subunit alpha (UniProt Accession No. P69905) (SEQ ID NO: 70), Haptoglobin (UniProt Accession No. P0073 8) (SEQ ID NO: 6), Hemopexin (UniProt Accession No. P02790) (SEQ ID NO: 39), Integrin alpha-Iib (UniProt Accession No. P08514) (SEQ ID NO: 54), Inter-alpha-trypsin inhibitor heavy chain H1 (UniProt Accession No. P19827) (SEQ ID NO: 63), Inter-alpha-trypsin inhibitor heavy chain H2 (UniProt Accession No. P19823) (SEQ ID NO: 62), Inter-alpha-trypsin inhibitor heavy chain H4 (UniProt Accession No. Q14624) (SEQ ID NO: 71), Plasma kallikrein (UniProt Accession No. P03952) (SEQ ID NO: 40), Apolipoprotein(a) (UniProt Accession No. P08519) (SEQ ID NO: 55), Leucine-rich alpha-2-glycoprotein (UniProt Accession No. P02750) (SEQ ID NO: 32), Lumican (UniProt Accession No. P51884) (SEQ ID NO: 69), Alpha-1-acid glycoprotein 1 (UniProt Accession No. P02763) (SEQ ID NO: 34), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), Peroxiredoxin-2 (UniProt Accession No. P32119) (SEQ ID NO: 66), Serum amyloid A-4 protein (UniProt Accession No. P35542) (SEQ ID NO: 67), Alpha-1-antitrypsin (UniProt Accession No. P01009) (SEQ ID NO: 12), Alpha-1-antichymotrypsin (UniProt Accession No. P01011) (SEQ ID NO: 13), Antithrombin-III (UniProt Accession No. P01008) (SEQ ID NO: 11), Heparin cofactor 2 (UniProt Accession No. P05546) (SEQ ID NO: 50), Plasma protease C1 inhibitor (UniProt Accession No. P05155) (SEQ ID NO: 48), Serotransferrin (UniProt Accession No. P02787) (SEQ ID NO: 38), Vitronectin (UniProt Accession No. P04004) (SEQ ID NO: 42), von Willebrand factor (UniProt Accession No. P04275) (SEQ ID NO: 45), or combinations thereof. In some embodiments, the disease is vascular disease and the proteins are any one or more of Angiotensinogen (UniProt Accession No. P01019) (SEQ ID NO: 14), Alpha-2-HS-glycoprotein (UniProt Accession No. P02765) (SEQ ID NO: 35), Apolipoprotein A-I UniProt Accession No. P02647) (SEQ ID NO: 21), Apolipoprotein A-II (UniProt Accession No.) (SEQ ID NO: 23), Apolipoprotein A-IV (UniProt Accession No. P06727) (SEQ ID NO: 52), Apolipoprotein B-100 (UniProt Accession No. P04114) (SEQ ID NO: 43), Apolipoprotein C-I (UniProt Accession No. P02654) (SEQ ID NO: 24), Apolipoprotein C-II (UniProt Accession No. P02655) (SEQ ID NO: 25), Apolipoprotein C-III (UniProt Accession No. P02656) (SEQ ID NO: 26), Apolipoprotein E (UniProt Accession No. P02649) (SEQ ID NO: 22), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Prothrombin (UniProt Accession No. P00734) (SEQ ID NO: 4), Hemoglobin subunit alpha (UniProt Accession No. P69905) (SEQ ID NO: 70), Haptoglobin (UniProt Accession No. P00738) (SEQ ID NO: 6), Integrin alpha-Iib (UniProt Accession No. P08514) (SEQ ID NO: 54), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), Peroxiredoxin-2 (UniProt Accession No. P32119) (SEQ ID NO: 66), Antithrombin-III (UniProt Accession No. P01008) (SEQ ID NO: 11), Heparin cofactor 2 (UniProt Accession No. P05546) (SEQ ID NO: 50), von Willebrand factor (UniProt Accession No. P04275) (SEQ ID NO: 45), or combinations thereof. In some embodiments, the disease is lung damage and the proteins are any one or more of Alpha-2-HS-glycoprotein (UniProt Accession No. P02765) (SEQ ID NO: 35), Serum albumin (UniProt Accession No. P02768) (SEQ ID NO: 36), Protein AMBP (UniProt Accession No. P02760) (SEQ ID NO: 33), Zinc-alpha-2-glycoprotein (UniProt Accession No. P25311) (SEQ ID NO: 64), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Clusterin (UniProt Accession No. P10909) (SEQ ID NO: 61), Vitamin D-binding protein (UniProt Accession No. P02774) (SEQ ID NO: 37), Hemoglobin subunit alpha (UniProt Accession No. P69905) (SEQ ID NO: 70), Plasma protease C1 inhibitor (UniProt Accession No. P05155) (SEQ ID NO; 48), Serotransferrin (UniProt Accession No. P02787) (SEQ ID NO: 38), von Willebrand factor (UniProt Accession No. P04275) (SEQ ID NO: 45), or combinations thereof. In some embodiments, the disease is lung damage and the proteins are any one or more of Alpha-2-HS-glycoprotein (UniProt Accession No. P02765) (SEQ ID NO: 35), Serum albumin (UniProt Accession No. P02768) (SEQ ID NO: 36), Protein AMBP (UniProt Accession No. P02760) (SEQ ID NO: 33), Zinc-alpha-2-glycoprotein (UniProt Accession No. P25311) (SEQ ID NO: 64), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Clusterin (UniProt Accession No. P10909) (SEQ ID NO: 61), C-reactive protein (UniProt Accession No. P02741) (SEQ ID NO: 28), Coagulation factor XII (UniProt Accession No. P00748 (SEQ ID NO: 9) Vitamin D-binding protein (UniProt Accession No. P02774) (SEQ ID NO: 37), Hemoglobin subunit alpha (UniProt Accession No. P69905) (SEQ ID NO: 70), Plasma protease C1 inhibitor (UniProt Accession No. P05155) (SEQ ID NO; 48), Serotransferrin (UniProt Accession No. P02787) (SEQ ID NO: 38), von Willebrand factor (UniProt Accession No. P04275) (SEQ ID NO: 45), or combinations thereof.

In various embodiments, the present invention provides a method for obtaining a protein biomarker signature for a subject, the method comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; and correlating the one or more peptides to one or more proteins so as to obtain the protein biomarker signature for the subject, wherein the one or more proteins are listed in Table 15. In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 5. In some embodiments, the mass spectrometry data comprises one or more Q1/Q3 mass value pairs, wherein the Q1/Q3 mass value pairs are correlated to the one or more peptides, and the one or more peptides are correlated to the one or more proteins according to Table 5. In some embodiments, the one or more peptides are correlated to the one or more proteins according to Table 7. In some embodiments, the mass spectrometry data comprises one or more precursor ions and one or more product ions, wherein the precursor ions and the product ions are correlated to the one or more peptides, and the one or more peptides are correlated to the one or more proteins according to Table 7. In some embodiments, the one or more peptides are correlated to the one or more proteins according to Table 8. In some embodiments, the mass spectrometry data comprises one or more precursor ions and one or more product ions, wherein the precursor ions and the product ions are correlated to the one or more peptides, and the one or more peptides are correlated to the one or more proteins according to Table 8. In some embodiments, the mass spectrometry is parallel reaction monitoring (PRM) mass spectrometry. In some embodiments, the one or more peptides are correlated to the one or more proteins according to Table 9. In some embodiments, the mass spectrometry data comprises one or more precursor ions and one or more product ions, wherein the precursor ions and the product ions are correlated to the one or more peptides, and the one or more peptides are correlated to the one or more proteins according to Table 9. In some embodiments, the one or more peptides are correlated to the one or more proteins according to Table 10. In some embodiments, the mass spectrometry data comprises one or more precursor ions and one or more product ions, wherein the precursor ions and the product ions are correlated to the one or more peptides, and the one or more peptides are correlated to the one or more proteins according to Table 10. In some embodiments, the mass spectrometry is data dependent acquisition (DDA) mass spectrometry. In some embodiments, the one or more peptides are correlated to the one or more proteins according to Table 11. In some embodiments, the mass spectrometry data comprises one or more precursor ions, wherein the precursor ions are correlated to the one or more peptides, and the one or more peptides are correlated to the one or more proteins according to Table 11. In some embodiments, the one or more peptides are correlated to the one or more proteins according to Table 12. In some embodiments, the mass spectrometry data comprises one or more precursor ions and one or more product ions, wherein the precursor ions and the product ions are correlated to the one or more peptides, and the one or more peptides are correlated to the one or more proteins according to Table 12. In some embodiments, the mass spectrometry is data independent acquisition (DIA) mass spectrometry. In some embodiments, the one or more peptides are correlated to the one or more proteins according to Table 13. In some embodiments, the the mass spectrometry data comprises one or more precursor ions and one or more product ions, wherein the precursor ions and the product ions are correlated to the one or more peptides, and the one or more peptides are correlated to the one or more proteins according to Table 13. In some embodiments, the one or more peptides are correlated to the one or more proteins according to Table 14. In some embodiments, the mass spectrometry data comprises one or more precursor ions and one or more product ions, wherein the precursor ions and the product ions are correlated to the one or more peptides, and the one or more peptides are correlated to the one or more proteins according to Table 14. In some embodiments, the one or more proteins are biomarkers for one or more functions or combination thereof. In some embodiments, the functions are inflammatory response, lipid metabolism, redox signaling, immune response, or endothelial dysfunction. In some embodiments, the functions are lipid metabolism, immune response, coagulation, inflammatory response, or redox signaling. In some embodiments, the function is inflammatory response and the proteins are any one or more of Alpha-2-macroglobulin (UniProt Accession No. P01023) (SEQ ID NO: 15), Angiotensinogen (UniProt Accession No. P01019) (SEQ ID NO: 14), Alpha-2-HS-glycoprotein (UniProt Accession No. P02765) (SEQ ID NO: 35), Apolipoprotein A-I (UniProt Accession No. P02647) (SEQ ID NO: 21), Apolipoprotein E (UniProt Accession No. P02649) (SEQ ID NO: 22), Complement C3 (UniProt Accession No. P01024) (SEQ ID NO: 16), Complement C5 (UniProt Accession No. P01031) (SEQ ID NO: 17), Complement factor H (UniProt Accession No. P08603) (SEQ ID NO: 56), C-reactive protein (UniProt Accession No. P02741) (SEQ ID NO: 28), Coagulation factor XII (UniProt Accession No. P00748) (SEQ ID NO: 9), Prothrombin (UniProt Accession No. P00734) (SEQ ID NO: 4), Vitamin D-binding protein (UniProt Accession No. P02774) (SEQ ID NO: 37), Gelsolin (UniProt Accession No. P06396) (SEQ ID NO: 51), Haptoglobin (UniProt Accession No. P00738) (SEQ ID NO: 6), Ig mu chain C region (UniProt Accession No. P01871) (SEQ ID NO: 19), Plasma kallikrein (UniProt Accession No. P03952) (SEQ ID NO: 40), Apolipoprotein(a) (UniProt Accession No. P08519) (SEQ ID NO: 55), Lumican (UniProt Accession No. P51884) (SEQ ID NO: 69), Alpha-1-acid glycoprotein 1 (UniProt Accession No. P02763) (SEQ ID NO: 34), Plasminogen (UniProt Accession No. P00747) (SEQ ID NO: 8), Serum paraoxonase/arylesterase 1 (UniProt Accession No. P27169) (SEQ ID NO: 65), Alpha-1-antitrypsin (UniProt Accession No. P01009) (SEQ ID NO: 12), Alpha-1-antichymotrypsin (UniProt Accession No. P01011) (SEQ ID NO: 13), Plasma protease C1 inhibitor (UniProt Accession No. P05155) (SEQ ID NO: 48), Vitronectin (UniProt Accession No. P04004) (SEQ ID NO: 42) or combinations thereof. In some embodiments, the function is lipid metabolism and the proteins are any one or more of Angiotensinogen (UniProt Accession No. P01019) (SEQ ID NO: 14), Alpha-2-HS-glycoprotein (UnitProt Accession No. P02765) (SEQ ID NO: 35), Serum albumin (UniProt Accession No. P02768) (SEQ ID NO: 36), Apolipoprotein A-I (UniProt Accession No. P02647) (SEQ ID NO: 21), Apolipoprotein A-II (UniProt Accession No. P02652) (SEQ ID NO: 23), Apolipoprotein A-IV (UniProt Accession No. P06727) (SEQ ID NO: 52), Apolipoprotein B-100 (UniProt Accession No. P04114) (SEQ ID NO: 43), Apolipoprotein C-I (UniProt Accession No. P02654) (SEQ ID NO: 24), Apolipoprotein C-III (UniProt Accession No. P02656) (SEQ ID NO: 26), Apolipoprotein E (UniProt Accession No. P02649) (SEQ ID NO: 22), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Complement factor H (UniProt Accession No. P08603) (SEQ ID NO: 56), Clusterin (UniProt Accession No. P10909) (SEQ ID NO: 61), C-reactive protein (UniProt Accession No. P02741) (SEQ ID NO: 28), Prothrombin (UniProt Accession No. P00734) (SEQ ID NO: 4), Vitamin D-binding protein (UniProt Accession No. P02774) (SEQ ID NO: 37), Haptoglobin (UniProt Accession No. P00738) (SEQ ID NO. 6), Plasma kallikrein (UniProt Accession No. P03952) (SEQ ID NO: 40), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), sex hormone binding globulin (UniProt Accession No. P04278) (SEQ ID NO: 46) or combinations thereof. In some embodiments, the function is redox signaling and the proteins are any one or more of Angiotensinogen (UniProt Accession No. P01019) (SEQ ID NO: 14), Serum albumin (UniProt Accession No. P02768) (SEQ ID NO: 36), Apolipoprotein A-I (UniProt Accession No. P02647) (SEQ ID NO: 21), Apolipoprotein A-IV (UniProt Accession No. P06727) (SEQ ID NO: 52), Apolipoprotein C-II (UniProt Accession No. P02655) (SEQ ID NO: 25), Apolipoprotein E (UniProt Accession No. P02649) (SEQ ID NO: 22), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Complement factor H (UniProt Accession No. P08603) (SEQ ID NO: 56), C-reactive protein (UniProt Accession No. P02741) (SEQ ID NO: 28), Prothrombin (UniProt Accession No. P00734) (SEQ ID NO: 4), Gelsolin (UniProt Accession No. P06396) (SEQ ID NO: 51), Hemoglobin subunit alpha (UniProt Accession No. P69905) (SEQ ID NO: 70), Haptoglobin (UniProt Accession No. P00738) (SEQ ID NO: 6), Inter-alpha-trypsin inhibitor heavy chain H4 (UniProt Accession No. Q14624) (SEQ ID NO: 71), Serum paraoxonase/lactonase 3 (UniProt Accession No. Q15166) (SEQ ID NO: 72), Peroxiredoxin-2 (UniProt Accession No. P32119) (SEQ ID NO: 66), Alpha-1-antitrypsin (UniProt Accession No. P01009) (SEQ ID NO: 12), Alpha-1-antichymotrypsin (UniProt Accession No. P01011) (SEQ ID NO: 13), Serotransferrin (UniProt Accession No. P02787) (SEQ ID NO: 38), or combinations thereof. In some embodiments, the function is immune response and the proteins are any one or more of Complement C1q subcomponent subunit B (UniProt Accession No. P02746) (SEQ ID NO: 29), Complement C1q subcomponent subunit C (UniProt Accession No. P02747) (SEQ ID NO: 30), Complement C1r subcomponent (UniProt Accession No. P00736) (SEQ ID NO: 50), Complement C1s subcomponent (UniProt Accession No. P09871) (SEQ ID NO: 58), Complement C3 (UniProt Accession No. P01024) (SEQ ID NO: 16), Complement C4-A (UniProt Accession No. P0C0L4) (SEQ ID NO: 59), Complement C5 (UniProt Accession No. P01031) (SEQ ID NO: 17), Complement component C8 alpha chain (UniProt Accession No. P07357) (SEQ ID NO: 53), Complement component C9 (UniProt Accession No. P02748) (SEQ ID NO: 31), Complement factor B (UniProt Accession No. P00751) (SEQ ID NO: 10), Plasma protease C1 inhibitor (UniProt Accession No. P05155) (SEQ ID NO: 48), or combinations thereof. In some embodiments, the function is endothelial dysfunction and the proteins are any one or more of Angiotensinogen (UniProt Accession No. P01019) (SEQ ID NO: 14), C-reactive protein (UniProt Accession No. P02741) (SEQ ID NO: 28), Lumican (UniProt Accession No. P51884) (SEQ ID NO: 69), Plasminogen (UniProt Accession No. P00747) (SEQ ID NO: 8), Peroxiredoxin-2 (UniProt Accession No. P32119) (SEQ ID NO: 66), Alpha-1-antitrypsin (UniProt Accession No. P01009) (SEQ ID NO: 12), Alpha-1-antichymotrypsin (UniProt Accession No. P01011) (SEQ ID NO: 13), Antithrombin-III (UniProt Accession No. P01008) (SEQ ID NO: 11), or combinations thereof. In some embodiments, the function is coagulation and the proteins are any one or more of Serum albumin (UniProt Accession No. P02768) (SEQ ID NO: 36), Apolipoprotein E (UniProt Accession No. P02649) (SEQ ID NO: 22), Complement C3 (UniProt Accession No. P01024) (SEQ ID NO: 16), Complement C4-A (UniProt Accession No. P0C0L4) (SEQ ID NO: 59), Complement C4-B (UniProt Accession No. P0C0L5) (SEQ ID NO: 60), Complement C5 (UniProt Accession No. P01031) (SEQ ID NO: 17), Complement component C9 (UniProt Accession No. P02748) (SEQ ID NO: 31), Complement factor B (UniProt Accession No. P00751) (SEQ ID NO: 10), Complement factor H (UniProt Accession No.) (SEQ ID NO: 56), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Fibrinogen (UniProt Accession No. P02671) (SEQ ID NO: 27), Haptoglobin (UniProt Accession No. P00738) (SEQ ID NO: 6), Integrin alpha-Iib (UniProt Accession No. P08514) (SEQ ID NO: 54), Plasma kallikrein (UniProt Accession No. P03952) (SEQ ID NO: 40), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Peroxiredoxin-2 (UniProt Accession No. P32119) (SEQ ID NO: 66), Plasma serine protease inhibitor (UniProt Accession No. P05154) (SEQ ID NO: 47), Antithrombin-III (UniProt Accession No. P01008) (SEQ ID NO: 11), Heparin cofactor 2 (UniProt Accession No. P05546) (SEQ ID NO: 50), Alpha-2-antiplasmin (UniProt Accession No. P08697) (SEQ ID NO: 57), Vitronectin (UniProt Accession No. P04004) (SEQ ID NO: 42), von Willebrand factor (UniProt Accession No. P04275) (SEQ ID NO: 45), or combinations thereof. In some embodiments, the function is lipid metabolism and the proteins are any one or more of Angiotensinogen (UniProt Accession No. P01019) (SEQ ID NO: 14), Alpha-2-HS-glycoprotein (UnitProt Accession No. P02765) (SEQ ID NO: 35), Serum albumin (UniProt Accession No. P02768) (SEQ ID NO: 36), Apolipoprotein A-I (UniProt Accession No. P02647) (SEQ ID NO: 21), Apolipoprotein A-II (UniProt Accession No. P02652) (SEQ ID NO: 23), Apolipoprotein A-IV (UniProt Accession No. P06727) (SEQ ID NO: 52), Apolipoprotein B-100 (UniProt Accession No. P04114) (SEQ ID NO: 43), Apolipoprotein C-I (UniProt Accession No. P02654) (SEQ ID NO: 24), Apolipoprotein C-III (UniProt Accession No. P02656) (SEQ ID NO: 26), Apolipoprotein E (UniProt Accession No. P02649) (SEQ ID NO: 22), Zinc-alpha-2-glycoprotein (UniProt Accession No. P25311) (SEQ ID NO: 64), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Complement factor H (UniProt Accession No. P08603) (SEQ ID NO: 56), Clusterin (UniProt Accession No. P10909) (SEQ ID NO: 61), C-reactive protein (UniProt Accession No. P02741) (SEQ ID NO: 28), Prothrombin (UniProt Accession No. P00734) (SEQ ID NO: 4), Vitamin D-binding protein (UniProt Accession No. P02774) (SEQ ID NO: 37), Haptoglobin (UniProt Accession No. P00738) (SEQ ID NO. 6), Plasma kallikrein (UniProt Accession No. P03952) (SEQ ID NO: 40), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), Alpha-1-antitrypsin (UniProt Accession No. P01009) (SEQ ID NO: 12), sex hormone binding globulin (UniProt Accession No. P04278) (SEQ ID NO: 46) or combinations thereof. In some embodiments, the function is inflammatory response and the proteins are any one or more of Alpha-2-macroglobulin (UniProt Accession No. P01023) (SEQ ID NO: 15), Angiotensinogen (UniProt Accession No. P01019) (SEQ ID NO: 14), Alpha-2-HS-glycoprotein (UniProt Accession No. P02765) (SEQ ID NO: 35), Apolipoprotein A-I (UniProt Accession No. P02647) (SEQ ID NO: 21), Apolipoprotein E (UniProt Accession No. P02649) (SEQ ID NO: 22), Complement C3 (UniProt Accession No. P01024) (SEQ ID NO: 16), Complement C5 (UniProt Accession No. P01031) (SEQ ID NO: 17), Complement factor H (UniProt Accession No. P08603) (SEQ ID NO: 56), C-reactive protein (UniProt Accession No. P02741) (SEQ ID NO: 28), C-reactive protein (UniProt Accession No. P02741) (SEQ ID NO: 28), Coagulation factor XII (UniProt Accession No. P00748) (SEQ ID NO: 9), Prothrombin (UniProt Accession No. P00734) (SEQ ID NO: 4), Vitamin D-binding protein (UniProt Accession No. P02774) (SEQ ID NO: 37), Gelsolin (UniProt Accession No. P06396) (SEQ ID NO: 51), Haptoglobin (UniProt Accession No. P00738) (SEQ ID NO: 6), Ig mu chain C region (UniProt Accession No. P01871) (SEQ ID NO: 19), Plasma kallikrein (UniProt Accession No. P03952) (SEQ ID NO: 40), Apolipoprotein(a) (UniProt Accession No. P08519) (SEQ ID NO: 55), Lumican (UniProt Accession No. P51884) (SEQ ID NO: 69), Alpha-1-acid glycoprotein 1 (UniProt Accession No. P02763) (SEQ ID NO: 34), Plasminogen (UniProt Accession No. P00747) (SEQ ID NO: 8), Serum paraoxonase/arylesterase 1 (UniProt Accession No. P27169) (SEQ ID NO: 65), Alpha-1-antitrypsin (UniProt Accession No. P01009) (SEQ ID NO: 12), Alpha-1-antichymotrypsin (UniProt Accession No. P01011) (SEQ ID NO: 13), Plasma protease C1 inhibitor (UniProt Accession No. P05155) (SEQ ID NO: 48), Vitronectin (UniProt Accession No. P04004) (SEQ ID NO: 42) or combinations thereof. In some embodiments, the function is immune response and the proteins are any one or more of Angiotensinogen (UniProt Accession No. P01019) (SEQ ID NO: 14), Alpha-2-HS-glycoprotein (UniProt Accession No. P02765) (SEQ ID NO: 35), Apolipoprotein A-I (UniProt Accession No. P02647) (SEQ ID NO: 21), Apolipoprotein A-II (UniProt Accession No. P02652) (SEQ ID NO: 23), Apolipoprotein E (UniProt Accession No. P02649) (SEQ ID NO: 22), Complement C1q subcomponent subunit B (UniProt Accession No. P02746) (SEQ ID NO: 29), Complement C1q subcomponent subunit C (UniProt Accession No. P02747) (SEQ ID NO: 30), Complement C1r subcomponent (UniProt Accession No. P00736) (SEQ ID NO: 50), Complement C1s subcomponent (UniProt Accession No. P09871) (SEQ ID NO: 58), Complement C3 (UniProt Accession No. P01024) (SEQ ID NO: 16), Complement C4-A (UniProt Accession No. P0C0L4) (SEQ ID NO: 59), Complement C4-B (UniProt Accession No. P0C0L5) (SEQ ID NO: 60), Complement C5 (UniProt Accession No. P01031) (SEQ ID NO: 17), Complement component C8 alpha chain (UniProt Accession No. P07357) (SEQ ID NO: 53), Complement component C9 (UniProt Accession No. P02748) (SEQ ID NO: 31), Complement factor B (UniProt Accession No. P00751) (SEQ ID NO: 10), Complement factor H (UniProt Accession No. P08603) (SEQ ID NO: 56), C-reactive protein (UniProt Accession No. P02741) (SEQ ID NO: 28), Coagulation factor X (UniProt Accession No. P00742) (SEQ ID NO: 7), Gelsolin (UniProt Accession No. P06396) (SEQ ID NO: 51), Ig alpha-1 chain C region (UniProt Accession No. P01876) (SEQ ID NO: 20), Ig gamma-3 chain C region (UniProt Accession No. P01860) (SEQ ID NO: 18), Ig mu chain C region (UniProt Accession No. P01871) (SEQ ID NO: 19), Lumican (UniProt Accession No. P51884) (SEQ ID NO: 69), Serum paraoxonase/arylesterase 1 (UniProt Accession No. P27169) (SEQ ID NO: 65), Plasma protease C1 inhibitor (UniProt Accession No. P05155) (SEQ ID NO: 48), Vitronectin (UniProt Accession No. P04004) (SEQ ID NO:), von Willebrand factor (UniProt Accession No. P04275) (SEQ ID NO: 45), or combinations thereof. In some embodiments, the one or more proteins are biomarkers for one or more diseases or combination thereof. In some embodiments, the disease is cardiovascular disease. In some embodiments, the disease is cardiovascular disease and the proteins are any one or more of Angiotensinogen (UniProt Accession No. P01019) (SEQ ID NO: 14), Apolipoprotein A-I (UniProt Accession No. P02647) (SEQ ID NO: 21), Apolipoprotein B-100 (UniProt Accession No. P04114) (SEQ ID NO: 43), Apolipoprotein E (UniProt Accession No. P02649) (SEQ ID NO: 22), Complement C3 (UniProt Accession No. P01024) (SEQ ID NO: 16), Complement C4-A (UniProt Accession No. P0C0L4) (SEQ ID NO: 59), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Gelsolin (UniProt Accession No. P06396) (SEQ ID NO: 51), Integrin alpha-Iib (UniProt Accession No. P08514) (SEQ ID NO: 54), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Antithrombin-III (UniProt Accession No. P01008) (SEQ ID NO: 11), Plasma protease C1 inhibitor (UniProt Accession No. P05155) (SEQ ID NO: 48), von Willebrand factor (UniProt Accession No. P04275) (SEQ ID NO: 45), or combinations thereof. In some embodiments, the disease is cardiovascular disease and the proteins are any one or more of Angiotensinogen (UniProt Accession No. P01019) (SEQ ID NO: 14), Apolipoprotein A-I (UniProt Accession No. P02647) ((SEQ ID NO: 21), Apolipoprotein B-100 (UniProt Accession No. P04114) (SEQ ID NO: 43), Apolipoprotein E (UniProt Accession No. P02649) (SEQ ID NO: 22), Complement C3 (UniProt Accession No. P01024) (SEQ ID NO: 16), Complement C4-A (UniProt Accession No. P0C0L4) (SEQ ID NO: 59), Complement C4-B (UniProt Accession No. P0C0L5) (SEQ ID NO: 60), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Gelsolin (UniProt Accession No. P06396) (SEQ ID NO: 51), Integrin alpha-Iib (UniProt Accession No. P08514) (SEQ ID NO: 54), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Antithrombin-III (UniProt Accession No. P01008) (SEQ ID NO: 11), Alpha-2-antiplasmin (UniProt Accession No. P08697) (SEQ ID NO: 57), Plasma protease C1 inhibitor (UniProt Accession No. P05155) (SEQ ID NO: 48), von Willebrand factor (UniProt Accession No. P04275) (SEQ ID NO: 45), or combinations thereof. In some embodiments, the cardiovascular disease is selected from congestive heart failure, arrhythmia, pericarditis, acute myocardial infarction, infarcted myocardium, coronary artery disease, coronary heart disease, ischemic heart disease, cardiomyopathy, stroke, hypertensive heart disease, heart failure, pulmonary heart disease, ischemic syndrome, coronary microvascular disease, cardiac dysrhythmias, rheumatic heart disease, aortic aneurysms, cardiomyopathy, atrial fibrillation, congenital heart disease, endocarditis, inflammatory heart disease, inflammatory cardiomegaly, myocarditis, valvular heart disease, cerebrovascular disease, peripheral artery disease or any combination thereof. In some embodiments, the disease is atherosclerosis and the proteins are any one or more of Angiotensinogen (UniProt Accession No. P01019) (SEQ ID NO: 14), Alpha-2-HS-glycoprotein (UniProt Accession No. P02765) (SEQ ID NO: 35), Apolipoprotein A-I (UniProt Accession No. P02647) (SEQ ID NO: 21), Apolipoprotein A-II (Uniprot Accession No. P02652) (SEQ ID NO: 23), Apolipoprotein A-IV (UniProt Accession No. P06727) (SEQ ID NO: 52), Apolipoprotein B-100 (UniProt Accession No. P04114) (SEQ ID NO: 43), Apolipoprotein C-I (UniProt Accession No. P02654) (SEQ ID NO: 24), Apolipoprotein C-II (UniProt Accession No. P02655) (SEQ ID NO: 25), Apolipoprotein (UniProt Accession No. P02656) (SEQ ID NO. 26), Apolipoprotein E (UniProt Accession No. P02649) (SEQ ID NO: 22), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Prothrombin (UniProt Accession No. P00734) (SEQ ID NO: 4), Hemoglobin subunit alpha (UniProt Accession No. P69905) (SEQ ID NO: 70), Haptoglobin (UniProt Accession No. P00738) (SEQ ID NO: 6), Integrin alpha-Iib (UniProt Accession No. P08514) (SEQ ID NO: 54), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), Peroxiredoxin-2 (UniProt Accession No. P32119) (SEQ ID NO: 66), Antithrombin-III (UniProt Accession No. P01008) (SEQ ID NO. 11), Heparin cofactor 2 (UniProt Accession No. P05546) (SEQ ID NO: 50), von Willebrand factor (UniProt Accession No. P04275) (SEQ ID NO: 45) or combinations thereof. In some embodiments, the disease is renal failure and the proteins are any one or more of Angiotensinogen (UniProt Accession No. P01019) (SEQ ID NO: 14), Serum albumin (UniProt Accession No. P02768) (SEQ ID NO: 36), Apolipoprotein B-100 (UniProt Accession No. P04114) (SEQ ID NO: 43), Complement C3 (UniProt Accession No. P01024) (SEQ ID NO: 16), Complement C4-A (UniProt Accession No. P0C0L4) (SEQ ID NO: 59), Complement C5 (UniProt Accession No. P01031) (SEQ ID NO: 17), Complement factor B (UniProt Accession No. P00751) (SEQ ID NO: 10), C-reactive protein (UniProt Accession No. P02741) (SEQ ID NO: 28), Coagulation factor X (UniProt Accession No. P00742) (SEQ ID NO: 7), Prothrombin (UniProt Accession No. P00734) (SEQ ID NO: 4), Hemoglobin subunit alpha (UniProt Accession No. P69905) (SEQ ID NO: 70), Antithrombin-III (UniProt Accession No. P01008) (SEQ ID NO: 11), Serotransferrin (UniProt Accession No. P02787) (SEQ ID NO: 38), or combinations thereof. In some embodiments, the disease is renal failure and the proteins are any one or more of Angiotensinogen (UniProt Accession No. P01019) (SEQ ID NO: 14), Serum albumin (UniProt Accession No. P02768) (SEQ ID NO: 36), Apolipoprotein B-100 (UniProt Accession No. P04114) (SEQ ID NO: 43), Complement C3 (UniProt Accession No. P01024) (SEQ ID NO: 16), Complement C4-A (UniProt Accession No. P0C0L4) (SEQ ID NO: 59), Complement C5 (UniProt Accession No. P01031) (SEQ ID NO: 17), Complement factor B (UniProt Accession No. P00751) (SEQ ID NO: 10), C-reactive protein (UniProt Accession No. P02741) (SEQ ID NO: 28), Coagulation factor X (UniProt Accession No. P00742) (SEQ ID NO: 7), Prothrombin (UniProt Accession No. P00734) (SEQ ID NO: 4), Hemoglobin subunit alpha (UniProt Accession No. P69905) (SEQ ID NO: 70), Antithrombin-III (UniProt Accession No. P01008) (SEQ ID NO: 11), Serotransferrin (UniProt Accession No. P02787) (SEQ ID NO: 38), or combinations thereof. In some embodiments, wherein the disease is liver disease and the proteins are any one or more of Alpha-1B-glycoprotein (UniProt Accession No. P04217) (SEQ ID NO: 44), Alpha-2-macroglobulin (UniProt Accession No. P01023) (SEQ ID NO: 15), Afamin (UniProt Accession No. P43652) (SEQ ID NO: 68), Angiotensinogen (UniProt Accession No. P01019) (SEQ ID NO: 14), Alpha-2-HS-glycoprotein (UniProt Accession No. P02765) (SEQ ID NO: 35), Serum albumin (UniProt Accession No. P02768) (SEQ ID NO: 36), Protein AMBP (UniProt Accession No. P02760) (SEQ ID NO: 33), Apolipoprotein A-I (UniProt Accession No. P02647) (SEQ ID NO: 21), Apolipoprotein A-II (UniProt Accession No. P02652) (SEQ ID NO: 23), Apolipoprotein B-100 (UniProt Accession No. P04114) (SEQ ID NO: 43), Apolipoprotein C-I (UniProt Accession No. P02654) (SEQ ID NO: 24), Apolipoprotein E (UniProt Accession No. P02649) (SEQ ID NO: 22), Apolipoprotein L1 (UniProt Accession No. 014791) (SEQ ID NO: 1), Complement C1q subcomponent subunit C (UniProt Accession No. P02747) (SEQ ID NO: 30), Complement C1r subcomponent (UniProt Accession No. P00736) (SEQ ID NO: 5), Complement C1s subcomponent (UniProt Accession No. P09871) (SEQ ID NO: 58), Complement C3 (UniProt Accession No. P01024) (SEQ ID NO: 16), Complement C4-A (UniProt Accession No. P0C0L4) (SEQ ID NO: 59), Complement C4-B (UniProt Accession No. P0C0L5) (SEQ ID NO: 60), C4b-binding protein alpha chain (UniProt Accession No. P04003) (SEQ ID NO: 41), Complement C5 (UniProt Accession No. P01031) (SEQ ID NO: 17), Complement component C8 alpha chain (UniProt Accession No. P07357) (SEQ ID NO: 53), Complement component C9 (UniProt Accession No. P02748) (SEQ ID NO: 31), Complement factor B (UniProt Accession No. P00751) (SEQ ID NO: 10), Complement factor H (UniProt Accession No. P08603) (SEQ ID NO: 56), Clusterin (UniProt Accession No. P10909) (SEQ ID NO: 61), Ceruloplasmin (UniProt Accession No. P00450) (SEQ ID NO: 2), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Fibrinogen (UniProt Accession No. P02671) (SEQ ID NO: 27), Vitamin D-binding protein (UniProt Accession No. P02774) (SEQ ID NO: 37), Gelsolin (UniProt Accession No. P06396) (SEQ ID NO: 51), Hemoglobin subunit alpha (UniProt Accession No. P69905) (SEQ ID NO: 70), Haptoglobin (UniProt Accession No. P0073 8) (SEQ ID NO: 6), Hemopexin (UniProt Accession No. P02790) (SEQ ID NO: 39), Integrin alpha-Iib (UniProt Accession No. P08514) (SEQ ID NO: 54), Inter-alpha-trypsin inhibitor heavy chain H1 (UniProt Accession No. P19827) (SEQ ID NO: 63), Inter-alpha-trypsin inhibitor heavy chain H2 (UniProt Accession No. P19823) (SEQ ID NO: 62), Inter-alpha-trypsin inhibitor heavy chain H4 (UniProt Accession No. Q14624) (SEQ ID NO: 71), Plasma kallikrein (UniProt Accession No. P03952) (SEQ ID NO: 40), Apolipoprotein(a) (UniProt Accession No. P08519) (SEQ ID NO: 55), Leucine-rich alpha-2-glycoprotein (UniProt Accession No. P02750) (SEQ ID NO: 32), Lumican (UniProt Accession No. P51884) (SEQ ID NO: 69), Alpha-1-acid glycoprotein 1 (UniProt Accession No. P02763) (SEQ ID NO: 34), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), Peroxiredoxin-2 (UniProt Accession No. P32119) (SEQ ID NO: 66), Serum amyloid A-4 protein (UniProt Accession No. P35542) (SEQ ID NO: 67), Alpha-1-antitrypsin (UniProt Accession No. P01009) (SEQ ID NO: 12), Alpha-1-antichymotrypsin (UniProt Accession No. P01011) (SEQ ID NO: 13), Antithrombin-III (UniProt Accession No. P01008) (SEQ ID NO: 11), Heparin cofactor 2 (UniProt Accession No. P05546) (SEQ ID NO: 50), Plasma protease C1 inhibitor (UniProt Accession No. P05155) (SEQ ID NO: 48), Serotransferrin (UniProt Accession No. P02787) (SEQ ID NO: 38), Vitronectin (UniProt Accession No. P04004) (SEQ ID NO: 42), von Willebrand factor (UniProt Accession No. P04275) (SEQ ID NO: 45), or combinations thereof. In some embodiments, the disease is vascular disease and the proteins are any one or more of Angiotensinogen (UniProt Accession No. P01019) (SEQ ID NO: 14), Alpha-2-HS-glycoprotein (UniProt Accession No. P02765) (SEQ ID NO: 35), Apolipoprotein A-I UniProt Accession No. P02647) (SEQ ID NO: 21), Apolipoprotein A-II (UniProt Accession No.) (SEQ ID NO: 23), Apolipoprotein A-IV (UniProt Accession No. P06727) (SEQ ID NO: 52), Apolipoprotein B-100 (UniProt Accession No. P04114) (SEQ ID NO: 43), Apolipoprotein C-I (UniProt Accession No. P02654) (SEQ ID NO: 24), Apolipoprotein C-11 (UniProt Accession No. P02655) (SEQ ID NO: 25), Apolipoprotein (UniProt Accession No. P02656) (SEQ ID NO: 26), Apolipoprotein E (UniProt Accession No. P02649) (SEQ ID NO: 22), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Prothrombin (UniProt Accession No. P00734) (SEQ ID NO: 4), Hemoglobin subunit alpha (UniProt Accession No. P69905) (SEQ ID NO: 70), Haptoglobin (UniProt Accession No. P00738) (SEQ ID NO: 6), Integrin alpha-Iib (UniProt Accession No. P08514) (SEQ ID NO: 54), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), Peroxiredoxin-2 (UniProt Accession No. P32119) (SEQ ID NO: 66), Antithrombin-III (UniProt Accession No. P01008) (SEQ ID NO: 11), Heparin cofactor 2 (UniProt Accession No. P05546) (SEQ ID NO: 50), von Willebrand factor (UniProt Accession No. P04275) (SEQ ID NO: 45), or combinations thereof. In some embodiments, the disease is lung damage and the proteins are any one or more of Alpha-2-HS-glycoprotein (UniProt Accession No. P02765) (SEQ ID NO: 35), Serum albumin (UniProt Accession No. P02768) (SEQ ID NO: 36), Protein AMBP (UniProt Accession No. P02760) (SEQ ID NO: 33), Zinc-alpha-2-glycoprotein (UniProt Accession No. P25311) (SEQ ID NO: 64), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Clusterin (UniProt Accession No. P10909) (SEQ ID NO: 61), Vitamin D-binding protein (UniProt Accession No. P02774) (SEQ ID NO: 37), Hemoglobin subunit alpha (UniProt Accession No. P69905) (SEQ ID NO: 70), Plasma protease C1 inhibitor (UniProt Accession No. P05155) (SEQ ID NO; 48), Serotransferrin (UniProt Accession No. P02787) (SEQ ID NO: 38), von Willebrand factor (UniProt Accession No. P04275) (SEQ ID NO: 45), or combinations thereof. In some embodiments, the disease is lung damage and the proteins are any one or more of Alpha-2-HS-glycoprotein (UniProt Accession No. P02765) (SEQ ID NO: 35), Serum albumin (UniProt Accession No. P02768) (SEQ ID NO: 36), Protein AMBP (UniProt Accession No. P02760) (SEQ ID NO: 33), Zinc-alpha-2-glycoprotein (UniProt Accession No. P25311) (SEQ ID NO: 64), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Clusterin (UniProt Accession No. P10909) (SEQ ID NO: 61), C-reactive protein (UniProt Accession No. P02741) (SEQ ID NO: 28), Coagulation factor XII (UniProt Accession No. P00748 (SEQ ID NO: 9) Vitamin D-binding protein (UniProt Accession No. P02774) (SEQ ID NO: 37), Hemoglobin subunit alpha (UniProt Accession No. P69905) (SEQ ID NO: 70), Plasma protease C1 inhibitor (UniProt Accession No. P05155) (SEQ ID NO; 48), Serotransferrin (UniProt Accession No. P02787) (SEQ ID NO: 38), von Willebrand factor (UniProt Accession No. P04275) (SEQ ID NO: 45), or combinations thereof. In some embodiments, the method further comprises comparing the biomarker signature from the subject to a biomarker signature from a reference sample. In some embodiments, the method further comprises making an assessment of the subject based on the comparison, wherein the assessment is a diagnosis of the disease in the subject. In some embodiments, the method further comprises treating the subject based on the diagnosis. In some embodiments, the reference sample is obtained from a control subject, wherein the control subject does not have a disease. In some embodiments, the reference sample is obtained from the subject before the subject is treated for the disease. In some embodiments, wherein the reference sample is from a subject that has been successfully treated for the disease. In some embodiments, the reference sample is from a subject that has the disease.

In various embodiments, the present invention provides a method for assessing and/or determining the state of health of a subject, the method comprising: obtaining a sample from a subject, contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; and correlating the one or more peptides to one or more proteins, wherein the one or more proteins are listed in Table 15; and; comparing the presence or level of the one or more proteins in the sample from the subject to the presence or level of the one or more proteins in a reference sample so as to assess and/or determine the state of health of the subject. In some embodiments, the presence of one or more proteins in the sample from the subject relative to the reference sample is indicative of a poor state of health. In some embodiments, an increase in the level of one or more proteins in the sample from the subject relative to the reference sample is indicative of a poor state of health. In some embodiments, a decrease in the level of one or more proteins in the sample from the subject relative to the reference sample is indicative of a poor state of health. In some embodiments, a change in the level of one or more proteins in the sample from the subject relative to the reference sample is indicative of a poor state of health. In some embodiments, the absence of one or more proteins in the sample for the subject relative to the reference sample is indicative of wellness. In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 5. In some embodiments, the mass spectrometry data comprises one or more Q1/Q3 mass value pairs, wherein the Q1/Q3 mass value pairs are correlated to the one or more peptides, and the one or more peptides are correlated to the one or more proteins according to Table 5. In some embodiments, the one or more peptides are correlated to the one or more proteins according to Table 7. In some embodiments, the mass spectrometry data comprises one or more precursor ions and one or more product ions, wherein the precursor ions and the product ions are correlated to the one or more peptides, and the one or more peptides are correlated to the one or more proteins according to Table 7. In some embodiments, the one or more peptides are correlated to the one or more proteins according to Table 8. In some embodiments, the mass spectrometry data comprises one or more precursor ions and one or more product ions, wherein the precursor ions and the product ions are correlated to the one or more peptides, and the one or more peptides are correlated to the one or more proteins according to Table 8. In some embodiments, the mass spectrometry is parallel reaction monitoring (PRM) mass spectrometry. In some embodiments, the one or more peptides are correlated to the one or more proteins according to Table 9. In some embodiments, the mass spectrometry data comprises one or more precursor ions and one or more product ions, wherein the precursor ions and the product ions are correlated to the one or more peptides, and the one or more peptides are correlated to the one or more proteins according to Table 9. In some embodiments, the one or more peptides are correlated to the one or more proteins according to Table 10. In some embodiments, the mass spectrometry data comprises one or more precursor ions and one or more product ions, wherein the precursor ions and the product ions are correlated to the one or more peptides, and the one or more peptides are correlated to the one or more proteins according to Table 10. In some embodiments, the mass spectrometry is data dependent acquisition (DDA) mass spectrometry. In some embodiments, the one or more peptides are correlated to the one or more proteins according to Table 11. In some embodiments, the mass spectrometry data comprises one or more precursor ions, wherein the precursor ions are correlated to the one or more peptides, and the one or more peptides are correlated to the one or more proteins according to Table 11. In some embodiments, the one or more peptides are correlated to the one or more proteins according to Table 12. In some embodiments, the mass spectrometry data comprises one or more precursor ions and one or more product ions, wherein the precursor ions and the product ions are correlated to the one or more peptides, and the one or more peptides are correlated to the one or more proteins according to Table 12. In some embodiments, the mass spectrometry is data independent acquisition (DIA) mass spectrometry. In some embodiments, the one or more peptides are correlated to the one or more proteins according to Table 13. In some embodiments, the mass spectrometry data comprises one or more precursor ions and one or more product ions, wherein the precursor ions and the product ions are correlated to the one or more peptides, and the one or more peptides are correlated to the one or more proteins according to Table 13. In some embodiments, the one or more peptides are correlated to the one or more proteins according to Table 14. In some embodiments, the mass spectrometry data comprises one or more precursor ions and one or more product ions, wherein the precursor ions and the product ions are correlated to the one or more peptides, and the one or more peptides are correlated to the one or more proteins according to Table 14. In various embodiments the present invention provides a method for diagnosing a disease in a subject, comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins so as to obtain the protein biomarker signature for the subject, wherein the one or more proteins are listed in Table 15: and comparing the protein biomarker signature from the subject to one or more reference protein biomarker signatures, wherein a change in the protein biomarker signature from the subject relative to one or more reference biomarker signatures is indicative of the disease in the subject In some embodiments, the method further comprises treating the subject and/or selecting a treatment for and/or providing a treatment to the subject based on the diagnosis. In various embodiments the present invention provides method for assessing the efficacy of the treatment, comprising: comparing the protein biomarker signature from the subject to one or more reference protein biomarker signatures, wherein a change in the protein biomarker signature from the subject relative to one or more reference biomarker signatures is indicative of the efficacy of the treatment. In some embodiments, the reference sample is obtained from a control subject, wherein the control subject does not have the disease In some embodiments, the reference sample is obtained from the subject before the subject is treated for the disease. In some embodiments, the reference sample is from a subject that has been successfully treated for the disease. In some embodiments, the disease is cardiovascular disease In some embodiments, the cardiovascular disease is selected from congestive heart failure, arrhythmia, pericarditis, acute myocardial infarction, infarcted myocardium, coronary artery disease, coronary heart disease, ischemic heart disease, cardiomyopathy, stroke, hypertensive heart disease, heart failure, pulmonary heart disease, ischemic syndrome, coronary microvascular disease, cardiac dysrhythmias, rheumatic heart disease, aortic aneurysms, cardiomyopathy, atrial fibrillation, congenital heart disease, endocarditis, inflammatory heart disease, inflammatory cardiomegaly, myocarditis, valvular heart disease, cerebrovascular disease, peripheral artery disease or any combination thereof. In some embodiments, the method further comprises determining that the subject does not have the disease; and selecting and/or providing a preventative treatment for the subject. In some embodiments, the method further comprises determining that the subject has the disease; and treating the subject and/or selecting a treatment for and/or providing a treatment for the subject. In various embodiments the present invention provides a method for assessing and/or determining the risk of developing a disease in a subject; comprising: obtaining a sample from the subject; treating the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins so as to obtain a protein biomarker signature for the subject, wherein the one or more proteins are listed in Table 15; and comparing the protein biomarker signature from the subject to one or more reference protein biomarker signatures, wherein a change in the protein biomarker signature from the subject relative to the reference protein biomarker signatures is indicative of an increased risk of the subject developing the disease. In some embodiments, the reference protein biomarker signature is obtained from a control subject, wherein the control subject does not have the disease. In some embodiments, the reference protein biomarker signature is obtained from the subject before the subject is treated for the disease. In some embodiments, the reference protein biomarker signature is from a subject that has been successfully treated for the disease. In some embodiments, the disease is cardiovascular disease. In some embodiments, the cardiovascular disease is selected from congestive heart failure, arrhythmia, pericarditis, acute myocardial infarction, infarcted myocardium, coronary artery disease, coronary heart disease, ischemic heart disease, cardiomyopathy, stroke, hypertensive heart disease, heart failure, pulmonary heart disease, ischemic syndrome, coronary microvascular disease, cardiac dysrhythmias, rheumatic heart disease, aortic aneurysms, cardiomyopathy, atrial fibrillation, congenital heart disease, endocarditis, inflammatory heart disease, inflammatory cardiomegaly, myocarditis, valvular heart disease, cerebrovascular disease, peripheral artery disease or any combination thereof.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1A-FIG. 1B depicts in accordance with various embodiments of the invention, automated proteomics sample preparation schema. FIG. 1A workflow and layout of the automated laboratory workstation. FIG. 1B outline of the automated digestion protocol. Detailed conditions are described in the online supplemental method provided in the examples section herein.

FIG. 2A-FIG. 2B depicts in accordance with various embodiments of the invention, reproducibility of the automated proteomics sample preparation workflow with expanded SRM analysis. Six plasma aliquots were processed on three separate days and analyzed with the IQ SRM-panel (a single scheduled 50 minutes LC MS/MS method targeting 72 proteins). FIG. 2A results stratified by average signal intensity from the three independent digests. FIG. 2B mean intensities (right panel) and mean CV % (left panel) of the three independent digests.

FIG. 3A-FIG. 3B depicts in accordance with various embodiments of the invention, trypsin digestion optimization. Reaction conditions were varied to optimize the digestion time FIG. 3A and trypsin:protein ratio FIG. 3B. FIG. 3A discloses SEQ ID NOS 251, 246, 245, 250, 248, 247, and 249, respectively, in order of appearance. FIG. 3B discloses SEQ ID NOS 79, 252, 138, 143, 81, 80, and 253, respectively, in order of appearance.

FIG. 4 depicts in accordance with various embodiments of the invention, inter-lab reproducibility. The final automated proteomics sample preparation method was implemented at Site 1 and at Site 2 sites. 24 or 48 plasma digests were analyzed on successive days at both labs. 93 peptides representing 44 proteins were monitored at both sites.

FIG. 5A-FIG. 5C depicts in accordance with various embodiments of the invention, FIG. 5A sample preparation reproducibility. β-gal was quantified by LC-SRM-MS in 16 samples prepared for SWATH-MS. FIG. 5B total proteins observed. FIG. 5C correlation of peptide and protein intensities in the SWATH-MS data (representative examples). FIG. 5A discloses SEQ ID NOS 254-259, respectively, in order of appearance.

FIG. 6A-FIG. 6C depicts in accordance with various embodiments of the invention, FIG. 6A represents a network map of the different function and/or disease hubs associated with the 72 plex. The brown circles in the edges represent the key hubs. The blue proteins in the edges represent proteins unique to a function and/or disease hub and the orange proteins in the center represent proteins shared across multiple functions and/or disease hubs. FIG. 6B is a table of the different key functions and/or disease hubs, their associated p-value, the molecule name and number of molecules in each hub associated with the 72 plex. FIG. 6C is a heatmap of the different proteins in the 72 plex and the degree of their overlap across the different function and/or disease hubs. For example, the proteins at the top C5, AGT, C3 in orange are shared across several functions/disease hubs and hence represent key regulators. The proteins at the bottom SERPIND1, SHBG, VTN in blue are unique to certain function/disease hub.

FIG. 7A-FIG. 7B depicts in accordance with various embodiments of the invention, automated proteomic sample preparation schema. FIG. 7A workflow and layout of the automated laboratory workstation. FIG. 7B outline of the automated digestion protocol. See examples section herein for details.

FIG. 8A-FIG. 8B depicts in accordance with various embodiments of the invention, reproducibility of the automated proteomic sample preparation workflow with a highly multiplexed SRM analysis. Six plasma aliquots were processed three times on separate days. The resulting peptides were analyzed by LC MS/MS with a 30 min LC gradient and a scheduled SRM method targeting 72 proteins. FIG. 8A average intensities (left panel) and interday % CVs (right panel) of the three independent digests. FIG. 8B results stratified by average signal intensity.

FIG. 9A-FIG. 9B depicts in accordance with various embodiments of the invention, trypsin digestion time course. Pooled human plasma was digested with trypsin for various times and then analyzed with the highly multiplexed SRM assay. Recoveries for each peptide were normalized to the 2 hour time point. FIG. 9A results for 162 peptides from 70 proteins are plotted individually (left panel) and collectively (right panel). FIG. 9B peak intensity variations over time for peptides from albumin, α-1-antitrypsin, and α-1-antichymotrypsin. FIG. 9B discloses SEQ ID NOS 166, 162, 160, 77, 76, 164, 165, 79, 110, 108, 109, 111, 112, 116, 114, 113, and 111 respectively, in order of appearance.

FIG. 10A-FIG. 10B depicts in accordance with various embodiments of the invention, interlab reproducibility. The final automated processing method was Site 1 (n=24) and at Site 2 (n=48). SRM assay results are presented for 360 transitions from 93 peptides representing 44 proteins that were measured at both sites. FIG. 10A percent CVs for individual peptides. FIG. 10B results stratified by % CV ranges.

FIG. 11A-FIG. 11B depicts in accordance with various embodiments of the invention, trypsin digestion optimization. Reaction conditions were varied to optimize FIG. 11A the typsin:protein ratio and FIG. 11B the digestion time. The results were analyzed with an SRM assay targeting endogenous proteins plus spiked β-gal. FIG. 11A discloses SEQ ID NOS 79, 128, 143, 81 and 253 respectively, in order of appearance. FIG. 11B discloses SEQ ID NOS 79, 78, 82, 81, and 73 respectively, in order of appearance.

FIG. 12A-FIG. 12D depicts in accordance with various embodiments of the invention, reproducibility assessed by the digestion of plasma samples from 48 individuals. β-gal was spiked into each sample before processing as a quality control indicator to report technical variation. The digests were analyzed with the highly multiplexed SRM assay. Signal intensity spread of peptides from β-gal (FIG. 12A) and selected endogenous proteins (FIG. 12B). % CVS (FIG. 12C). Average intensities (FIG. 12D). FIG. 12A discloses SEQ ID NOS 254, 255, 256, 258, and 75 respectively, in order of appearance. FIG. 12B discloses SEQ ID NOS 246, 1917, 1918, 247, 248, and 1919 respectively, in order of appearance. FIG. 12C discloses SEQ ID NOS 254, 255, 256, 258, 75, 246, 1917, 1918, 247, 248, and 1919 respectively, in order of appearance. FIG. 12D discloses SEQ ID NOS 254, 255, 258, 75, 246, 1917, 1918, 247, 248 and 1919 respectively, in order of appearance.

FIG. 13 depicts in accordance with various embodiments of the invention, quantifier transition validation. Transition ratios were determined to compare the measured amounts of qualifier and quantifier transitions for six exemplary peptides in 48 plasma samples. FIG. 13 discloses SEQ ID NOS 81, 80, 1920, 79, 82, and 125 respectively, in order of appearance.

FIG. 14A-FIG. 14B depicts in accordance with various embodiments of the invention, FIG. 14A is a Targeted Peptide Selection process to build the 72 plex assay starting with all 2529 Expected Peptides between 6-30 amino acids in length with no missed cleavages. We filtered the list to only keep 2274 peptides that match to a single protein or single gene family (considered to be proteotypic in order to perform unambiguous mapping of the measured protein analyte in the 72 plex). The resulting peptides were further filtered to only keep 1797 peptides that do not contain Methionine residue in the peptide sequence to avoid uncontrolled modification bias that often affects methionine. Of the total 1797 expected peptides, we have MS evidence for 1103 Peptides that have been observed using 1 or more MS methods (including 6500 MRM-MS, QE PRM-MS, 6600 DIA-MS, Lumos DDA-MS). FIG. 14B represents a Venn diagram of the 1103 observed peptides to depict the shared and unique peptides to each of the 4 MS methods used above.

FIG. 15 depicts in accordance with various embodiments of the invention, representative Extracted Ion Chromatogram (XIC) obtained by PRM analysis of peptide ELLESYIDGR (SEQ ID NO: 92) correlated to a protein Prothrombin (UniProt Accession No. P00734) (SEQ ID NO: 4). An overlay of the endogenous peptide XIC (red trace) and reference peptide XIC (blue trace) recorded during chromatographic elution profile (left panel). XICs are extracted from the main fragment ions of the endogenous peptide (center panel) and reference peptide (right panel). Stable isotope labelled peptide (heavy) is used to confirm the identity of the native peptide.

FIG. 16 depicts in accordance with various embodiments of the invention, representative Extracted Ion Chromatogram (XIC) obtained by PRM analysis of peptide YTTEIIK (SEQ ID NO: 95) correlated to a protein Complement C1r subcomponent (UniProt Accession No. P00736) (SEQ ID NO: 50). An overlay of the endogenous peptide XIC (red trace) and reference peptide XIC (blue trace) recorded during chromatographic elution profile (left panel). XICs are extracted from the main fragment ions of the endogenous peptide (center panel) and reference peptide (right panel). Stable isotope labelled peptide (heavy) is used to confirm the identity of the native peptide.

FIG. 17 depicts in accordance with various embodiments of the invention, representative Extracted Ion Chromatogram (XIC) obtained by PRM analysis of peptide EFGNTLEDK (SEQ ID NO: 145) correlated to a protein Apolipoprotein C-I (UniProt Accession No. P02654) (SEQ ID NO: 24). An overlay of the endogenous peptide XIC (red trace) and reference peptide XIC (blue trace) recorded during chromatographic elution profile (left panel). XICs are extracted from the main fragment ions of the endogenous peptide (center panel) and reference peptide (right panel). Stable isotope labelled peptide (heavy) is used to confirm the identity of the native peptide.

FIG. 18 depicts in accordance with various embodiments of the invention, representative Extracted Ion Chromatogram (XIC) obtained by PRM analysis of peptide IYLQPGR (SEQ ID NO: 224) correlated to a protein Inter-alpha-trypsin inhibitor heavy chain H2 (UniProt Accession No. P19823) (SEQ ID NO: 62). An overlay of the endogenous peptide XIC (red trace) and reference peptide XIC (blue trace) recorded during chromatographic elution profile (left panel). XICs are extracted from the main fragment ions of the endogenous peptide (center panel) and reference peptide (right panel). Stable isotope labelled peptide (heavy) is used to confirm the identity of the native peptide.

FIG. 19 depicts in accordance with various embodiments of the invention, representative Extracted Ion Chromatogram (XIC) obtained by PRM analysis of peptide SPDVINGSPISQK (SEQ ID NO: 209) correlated to a protein Complement factor H (UniProt Accession No. P08603) (SEQ ID NO: 56). An overlay of the endogenous peptide XIC (red trace) and reference peptide XIC (blue trace) recorded during chromatographic elution profile (left panel). XICs are extracted from the main fragment ions of the endogenous peptide (center panel) and reference peptide (right panel). Stable isotope labelled peptide (heavy) is used to confirm the identity of the native peptide.

FIG. 20 depicts in accordance with various embodiments of the invention, representative Extracted Ion Chromatogram (XIC) obtained by PRM analysis of peptide QFPILLDFK (SEQ ID NO: 193) correlated to a protein Heparin cofactor 2 (UniProt Accession No. P05546) (SEQ ID NO: 50). An overlay of the endogenous peptide XIC (red trace) and reference peptide XIC (blue trace) recorded during chromatographic elution profile (left panel). XICs are extracted from the main fragment ions of the endogenous peptide (center panel) and reference peptide (right panel). Stable isotope labelled peptide (heavy) is used to confirm the identity of the native peptide.

FIG. 21 depicts in accordance with various embodiments of the invention, representative Extracted Ion Chromatogram (XIC) obtained by PRM analysis of peptide FTPTETNK (SEQ ID NO: 344) correlated to a protein Complement component C9 (UniProt Accession No. P02748) (SEQ ID NO: 31). An overlay of XICs of the endogenous precursor ion and the main fragment ions recorded during chromatographic elution profile (left panel). A magnified view of the fragment ions XICs overlay form the left panel (right panel).

FIG. 22 depicts in accordance with various embodiments of the invention, representative Extracted Ion Chromatogram (XIC) obtained by PRM analysis of peptide TAAQNLYEK (SEQ ID NO: 146) correlated to a protein Apolipoprotein (UniProt Accession No. P02655) (SEQ ID NO: 25). An overlay of XICs of the endogenous precursor ion and the main fragment ions recorded during chromatographic elution profile (left panel). A magnified view of the fragment ions XICs overlay form the left panel (right panel).

FIG. 23 depicts in accordance with various embodiments of the invention, representative Extracted Ion Chromatogram (XIC) obtained by PRM analysis of peptide QQTEWQSGQR (SEQ ID NO: 492) correlated to a protein Apolipoprotein E (UniProt Accession No. P02649) (SEQ ID NO: 22). An overlay of XICs of the endogenous precursor ion and the main fragment ions recorded during chromatographic elution profile (left panel). A magnified view of the fragment ions XICs overlay form the left panel (right panel).

FIG. 24 depicts in accordance with various embodiments of the invention, representative Extracted Ion Chromatogram (XIC) obtained by PRM analysis of peptide NDLISATK (SEQ ID NO: 293) correlated to a protein Inter-alpha-trypsin inhibitor heavy chain H2 (UniProt Accession No. P19823) (SEQ ID NO: 62). An overlay of XICs of the endogenous precursor ion and the main fragment ions recorded during chromatographic elution profile (left panel). A magnified view of the fragment ions XICs overlay form the left panel (right panel).

FIG. 25 depicts in accordance with various embodiments of the invention, representative Extracted Ion Chromatogram (XIC) obtained by PRM analysis of peptide NVHSGSTFFK (SEQ ID NO: 1504) correlated to a protein Inter-alpha-trypsin inhibitor heavy chain H4 (UniProt Accession No. Q14624) (SEQ ID NO: 71). An overlay of XICs of the endogenous precursor ion and the main fragment ions recorded during chromatographic elution profile (left panel). A magnified view of the fragment ions XICs overlay form the left panel (right panel).

FIG. 26 depicts in accordance with various embodiments of the invention, representative Extracted Ion Chromatogram (XIC) obtained by PRM analysis of peptide NNQIDHIDEK (SEQ ID NO: 1526) correlated to a protein Lumican (UniProt Accession No. P51884) (SEQ ID NO: 69). An overlay of XICs of the endogenous precursor ion and the main fragment ions recorded during chromatographic elution profile (left panel). A magnified view of the fragment ions XICs overlay form the left panel (right panel).

FIG. 27A-FIG. 27C depicts in accordance with various embodiments of the invention, FIG. 27A represents a network map of the different function and/or disease hubs associated with the 72 plex. The brown circles in the edges represent the key hubs. The blue proteins in the edges represent proteins unique to a function and/or disease hub and the orange proteins in the center represent proteins shared across multiple functions and/or disease hubs. FIG. 27B is a table of the different key functions and/or disease hubs, the protein identification numbers from UniProt database, the protein names and gene names associated with the 72 plex. FIG. 27C is a heatmap of the different proteins in the 72 plex and the degree of their overlap across the different function and/or disease hubs. For example, the proteins at the top C5, CRP, APOE, C3, AGT in orange are shared across several functions/disease/process and hence represent key regulators.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application.

For references on mass spectrometry and proteomics, see e.g., Salvatore Sechi, Quantitative Proteomics by Mass Spectrometry (Methods in Molecular Biology) 2nd ed. 2016 Edition, Humana Press (New York, N.Y., 2009); Daniel Martins-de-Souza, Shotgun Proteomics: Methods and Protocols 2014 edition, Humana Press (New York, N.Y., 2014); Jörg Reinders and Albert Sickmann, Proteomics: Methods and Protocols (Methods in Molecular Biology) 2009 edition, Humana Press (New York, N.Y., 2009); and Jörg Reinders, Proteomics in Systems Biology: Methods and Protocols (Methods in Molecular Biology) 1^{st} ed. 2016 edition, Humana Press (New York, N.Y., 2009).

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

The term “sample” or “biological sample” as used herein denotes a sample taken or isolated from a biological organism, e.g., a tumor sample from a subject. Exemplary biological samples include, but are not limited to, cheek swab; mucus; whole blood, blood, serum; plasma; urine; saliva; semen; lymph; fecal extract; sputum; other body fluid or biofluid; cell sample; tissue sample; tumor sample; and/or tumor biopsy etc. Exemplary biological samples include, but are not limited to, cheek swab; mucus; whole blood, blood, serum; plasma; blood products, urine; saliva; semen; lymph; fecal extract; sputum; other body fluid or biofluid; cell sample; tissue sample; tissue extract; tumor sample; and/or tumor biopsy etc. The term also includes a mixture of the above-mentioned samples. The term “sample” also includes untreated or pretreated (or pre-processed) biological samples. In some embodiments, a sample can comprise one or more cells from the subject. In some embodiments, a sample can be a tumor cell sample, e.g. the sample can comprise cancerous cells, cells from a tumor, and/or a tumor biopsy. In some embodiments, samples or biological samples comprise derivatives of blood products, including blood, plasma and/or serum. In some embodiments, the sample is a biological sample. In some embodiments, the sample is blood. In some embodiments, the sample is plasma. In some embodiments, the sample is serum. In some embodiments the sample is cerebrospinal fluid (CSF). In some embodiments the sample is a tissue extract. In some embodiments the sample is a biopsy sample. In some embodiments the sample is a biopsy specimen. In some embodiments the sample is urine. In some embodiments the sample is plasma, serum, cerebrospinal fluid (CSF), a tissue extract, urine, a biopsy specimen, or a biopsy sample. In some embodiments, the sample comprises one or more proteins and/or one or more peptides. In some embodiments, the sample comprises one or more peptides. In some embodiments, the sample comprises one or more proteins.

The terms “body fluid” or “bodily fluids” are liquids originating from inside the bodies of organisms. Bodily fluids include amniotic fluid, aqueous humour, vitreous humour, bile, blood (e.g., serum), breast milk, cerebrospinal fluid, cerumen (earwax), chyle, chyme, endolymph and perilymph, exudates, feces, female ejaculate, gastric acid, gastric juice, lymph, mucus (e.g., nasal drainage and phlegm), pericardial fluid, peritoneal fluid, pleural fluid, pus, rheum, saliva, sebum (skin oil), serous fluid, semen, smegma, sputum, synovial fluid, sweat, tears, urine, vaginal secretion, and vomit. Extracellular bodily fluids include intravascular fluid (blood plasma), interstitial fluids, lymphatic fluid and transcellular fluid. Immunoglobulin G (IgG), the most abundant antibody subclass, may be found in all body fluids. “Biological sample” also includes a mixture of the above-mentioned body fluids. “Biological samples” may be untreated or pretreated (or pre-processed) biological samples.

Sample collection procedures and devices known in the art are suitable for use with various embodiment of the present invention. Examples of sample collection procedures and devices include but are not limited to: phlebotomy tubes (e.g., a vacutainer blood/specimen collection device for collection and/or storage of the blood/specimen), dried blood spots, Microvette CB300 Capillary Collection Device (Sarstedt), HemaXis blood collection devices (microfluidic technology, Hemaxis), Volumetric Absorptive Microsampling (such as CE-IVD Mitra microsampling device for accurate dried blood sampling (Neoteryx), HemaSpot™-HF Blood Collection Device. Additional sample collection procedures and devices include but are not limited to: a tissue sample collection device; standard collection/storage device (e.g., a collection/storage device for collection and/or storage of a sample (e.g., blood, plasma, serum, urine, etc.); a dried blood spot sampling device. In some embodiments, the Volumetric Absorptive Microsampling (VAMS™) samples can be stored and mailed, and an assay can be performed remotely.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. In various embodiments, the subject is human.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

As used herein, the term “amino acid” refers to naturally occurring and synthetic amino acids, as well as amino acid analogs and amino acid mimetics that operate in a manner similar to the naturally occurring amino acids. Naturally occurring amino acids are those encoded by the genetic code, as well as those amino acids that are later modified, e.g., hydroxyproline, -carboxyglutamate, and O-phosphoserine. Amino acid analogs refers to compounds that have the same basic chemical structure as a naturally occurring amino acid, i.e., an carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that operates in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A protein refers to any of a class of nitrogenous organic compounds that comprise large molecules composed of one or more long chains of amino acids and are an essential part of all living organisms. A protein may contain various modifications to the amino acid structure such as disulfide bond formation, phosphorylations and glycosylations. A linear chain of amino acid residues may be called a “polypeptide.” A protein contains at least one polypeptide. Short polypeptides, e.g., containing less than 20-30 residues, are sometimes referred to as “peptides.”

In some embodiments, the term “peptide” as used herein refers to a polymer of amino acid residues typically ranging in length from 2 to about 30, or to about 40, or to about 50, or to about 60, or to about 70 residues. In certain embodiments the peptide ranges in length from about 2, 3, 4, 5, 7, 9, 10, or 11 residues to about 60, 50, 45, 40, 45, 30, 25, 20, or 15 residues. In certain embodiments the peptide ranges in length from about 8, 9, 10, 11, or 12 residues to about 15, 20 or 25 residues. In certain embodiments the amino acid residues comprising the peptide are “L-form” amino acid residues, however, it is recognized that in various embodiments, “D” amino acids can be incorporated into the peptide. Peptides also include amino acid polymers in which one or more amino acid residues are an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an a-ester, a f3-ester, a thioamide, phosphonamide, carbamate, hydroxylate, and the like (see, e.g., Spatola, (1983) Chem. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn eta/., (1990) Pp. 969-970 in Proc. 11th American Peptide Symposium, ESCOM Science Publishers, The Netherlands, and the like)).

In some embodiments, the one or more proteins and/or one or more peptides in the sample are modified. In some embodiments, the one or more proteins and/or one or more peptides in the sample are chemically modified. In some embodiments, the modification is any one or more of phosphorylation, methylation, acetylation, o-GlycNacylation, s-nitrosylation, citrullination, sumoylation, ubiquitinylation, neddylation, methyglyoxylation, or a post-translational modification.

The term “threshold” as used herein refers to the magnitude or intensity that must be exceeded for a certain reaction, phenomenon, result, or condition to occur or be considered relevant. The relevance can depend on context, e.g., it may refer to a positive, reactive or statistically significant relevance.

The term “condition” (biological state or health state) is understood in the present invention as status of a subject that can be described by physical, mental or social criteria. It includes as well so-called “healthy” and “diseased” conditions, therefore it is not limited to the WHO definition of health as “a state of complete physical, mental, and social well-being and not merely the absence of disease or infirmity,” but includes disease and infirmity.

The term “disease” refers to an abnormal condition affecting the body of an organism. The term “disorder” refers to a functional abnormality or disturbance.

The term “state of health” includes at least one condition as defined herein. It may also include a plurality of different conditions. In some embodiments, the state of health is a healthy state. In some embodiments, the state of health is a diseased state. In some embodiments, the state of health is a poor state of health. In some embodiments, the state of health is a good state of health. In some embodiments, the state of health is a healthy state. In some embodiments, the state of health is an unhealthy state.

The term “poor state of health” means that the subject has one or more diseases or disorders or is at risk of having one or more diseases or disorders; has or is at risk of having one or more disorders associated with function or development; and/or has or is at risk of having one or more disorders of a function or development.

The term “good state of health” means that the subject does not have one or more diseases or disorders; does not have one or more disorders associated with function or development; or does not have one or more disorders of a function or development.

The term “healthy state” or “normal state” means that the state of a subject is not abnormal or does not comprise a disease or disorder.

A “healthy subject” or “normal subject” is a subject that does not have a disease or disorder.

The term “unhealthy state” or “abnormal state” means that the state of the subject is abnormal or does comprise a disease or disorder.

The term “unhealthy subject” or “abnormal subject” is a subject that does have a disease or disorder.

The term “wellness” means that the subject does not have one or more diseases or disorders; does not have one or more disorders associated with function or development; does not have one or more disorders of a function or development; or the subject is in a healthy state; or the subject is in a normal state; or the subject is in a state of being in good health.

Non-limiting examples of diseases include atherosclerosis, inflammatory response, cardiac disease, lung damage, renal failure, endothelial dysfunction, cardiovascular disease, neurological disease, organismal injury, organismal abnormalities, psychological disorders, developmental disorder, hereditary disorder, immunological disease, cell-to-cell signaling, cell-to-cell interaction, cardiac inflammation, connective tissue development and function, connective tissue disorders, skeletal disorders, muscular disorders.

Non-limiting examples of diseases include disorders associated with function or development such as lipid metabolism, redox signaling, immune response, hematological system development, hematological system function, reproductive system development, reproductive system function, and gene expression.

Other non-limiting examples of diseases include cancer, gastrointestinal disease, hepatic system disease, reproductive system disease, dermatological diseases, cell death, metabolic disease, neurological disease, immunological disease, hematological disease, psychological disorders, endocrine system disorders, connective tissue disorders, infectious diseases, hereditary disorder, respiratory disease, renal disease, urological disease, nutritional disease, and ophthalmic disease.

Other non-limiting examples of diseases include disorders associated with function or development such as cellular movement, cellular growth, cell proliferation, tissue morphology, organismal survival, molecular transport, immune cell trafficking, cell-to-cell signaling, cell-to-cell interaction, lipid metabolism, small molecule biochemistry, tissue development, protein synthesis, free radical scavenging, protein degradation, protein synthesis, hematological system development, hematological system function, tissue morphology, carbohydrate metabolism, cellular function and maintenance, cell signaling, vitamin metabolism, mineral metabolism, digestive system development, digestive system function, hepatic system development, hepatic system function, hair development, hair function, skin development, skin function, nervous system development, nervous system function, cellular compromise, nucleic acid metabolism, small molecule biochemistry, organ morphology, organismal development, renal system development, renal system function, urological system development, urological system function, humoral immune response, amino acid metabolism, energy production, post-translational modification.

Other non-limiting examples of diseases include Cardiovascular Disease, Hematological Disease, Hematological System Development and Function, Neurological Disease, Organismal Injury and Abnormalities, Psychological Disorders, Developmental Disorder, Hereditary Disorder, Immunological Disease, Organismal Injury and Abnormalities, Cell-To-Cell Signaling and Interaction, Reproductive System Development and Function, Gene Expression, Cardiac Inflammation, Cardiovascular Disease, Connective Tissue Development and Function, Connective Tissue Disorders, Organismal Injury and Abnormalities, Hereditary Disorder, Organismal Injury and Abnormalities, Skeletal and Muscular Disorders.

Other non-limiting examples of diseases include abdominal cancer, urogenital cancer, abdominal adenocarcinoma, liver lesion, tumorigenesis of genital organ, genital tumor, pelvic cancer, female genital tract cancer, tumorigenesis of reproductive tract, liver cancer, skin lesion, melanoma, skin tumor, malignant cutaneous melanoma cancer, cell death, breast or ovarian cancer, lymphohematopoietic cancer, amyloidosis, glucose metabolism disorder, Lymphoid Cancer and Tumors, hematologic cancer, Dementia, lymphoproliferative malignancy, lymphoid cancer, Alzheimer's disease, diabetes mellitus, inflammation of organ, inflammation of absolute anatomical region, Rheumatic Disease, leukemia, inflammation of body cavity, ovarian cancer, Viral Infection, myeloproliferative disorder, occlusion of artery, chronic inflammatory disorder, atherosclerosis, systemic autoimmune syndrome, acute leukemia, bone marrow cancer, inflammation of joint, neuromuscular disease, acute myeloid leukemia, Thrombosis, Infarction, Movement Disorders, hemostasis, acute coronary syndrome, rheumatoid arthritis, disorder of basal ganglia, autosomal, recessive disease, coronary disease, peripheral vascular disease, Huntington's Disease, abnormal morphology of abdomen, blood clot, immunodeficiency, myocardial infarction, cerebrovascular dysfunction, Bacterial Infections, endocytosis, advanced malignant tumor, damage of lung, hemorrhagic disease, coronary artery disease, Fibrosis, Hypertension, autosomal dominant disease, acute myocardial infarction, injury of lung, systemic inflammatory response syndrome and/or sepsis, infiltration by neutrophils, renal impairment, vascular lesion, anemia, progressive motor neuropathy, disorder of pregnancy, sepsis, atherosclerotic lesion, failure of kidney, chemotaxis of myeloid cells, chemotaxis of phagocytes, inflammation of liver, accumulation of lipid, inflammation of intestine, blood protein disorder, obesity, adenoma, psoriasis, necrosis of epithelial tissue, complement component deficiency, damage of genitourinary system, chronic kidney disease, accumulation of blood cells, insulin resistance, Nephritis, colitis, non-insulin-dependent diabetes mellitus, thrombus, hyperlipoproteinemia, hemolysis, acute lung injury, damage of kidney, dyslipidemia, stroke, lupus erythematosus, cytopenia, Edema, chronic fatigue syndrome, atherogenesis, ischemia of brain, chronic renal failure, thrombocytopenia, diabetic complication, Polycystic Kidney Disease, damage of liver, retinal degeneration, age-related, macular degeneration, dengue fever, formation of blood clot, severe sepsis, Hypercholesterolemia, cholangiocarcinoma, hyperlipidemia, inflammation of pancreas, stenosis, septic shock, cirrhosis of liver, damage of central nervous system, cell death of endothelial cells, Wound, injury of liver, glomerulonephritis, fibrin clot, dengue hemorrhagic fever, thromboembolism, ischemia of heart, thrombosis of vein, infarction of cerebrum, hemolytic anemia, prostatic intraepithelial neoplasia, damage of bone, end stage renal disease, Parasitic Infection, apoptosis of endothelial cells, abnormality of left ventricle, cardiac lesion, venous thromboembolism, fibrinolysis, systemic inflammatory response syndrome, familial amyloidosis, atherosclerosis of aorta, formation of thrombus, bleeding of gastrointestinal tract, Hypertriglyceridemia, hereditary bleeding disorder, autoimmune, glomerulonephritis, cerebral ischemia, infection by Herpesviridae, sexually transmitted disease, metabolic syndrome X, hereditary angioedema, deep vein thrombosis, familial hypercholesterolemia, opsonization, microcytic anemia, unstable angina, bleeding of skin, ischemic stroke, chronic pancreatitis, malaria, albuminuria, angina pectoris, vasculitis, degradation of connective tissue, cerebral malaria, thrombophilia, atypical hemolytic uremic syndrome, familial amyloidotic polyneuropathy, systemic amyloidosis, hypolipoproteinemia, ST-elevation myocardial infarction, focal necrosis of liver, mixed hyperlipidemia, Stevens-Johnson syndrome, pulmonary embolism, agammaglobulinemia, infection by cytomegalovirus, disorder of menstruation, abnormal morphology of mesangial matrix, injury of intestine, activation of vascular endothelial cells, thrombosis of artery, hip fracture, immune response of peritoneal macrophages, acquired immunodeficiency syndrome, autoimmune pancreatitis, damage of blood vessel, purpura, benign thyroid nodule, familial dementia, abdominal aortic aneurysm, recurrent venous thrombosis, hemorrhagic stroke, heparin-induced thrombocytopenia, Finnish type amyloidosis, placental insufficiency, portal vein thrombosis, injury of myocardium, non-ST elevation myocardial infarction, acute immune thrombocytopenic purpura, chronic immune thrombocytopenic purpura, congenital agammaglobulinemia, fetal erythroblastosis, hereditary thrombophilia, recurrent sinopulmonary infection, chronic idiopathic thrombocytopenic purpura, thrombosis of kidney, vascular injury, abnormal aggregation of blood platelets, acute pulmonary embolism, neonatal alloimmune, thrombocytopenia, whooping cough, congenital heart block, familial combined hyperlipidemia, Devic's syndrome, toxic epidermal necrolysis, hematoma, hyperplasia of vascular smooth muscle cells, Rasmussen's encephalitis, hyper-igm immunodeficiency syndrome, chronic inflammatory demyelinating polyradiculoneuropathy, microalbuminuria, acute phase atypical hemolytic uremic syndrome, bleeding of abdominal aorta, recurrent deep vein thrombosis, recurrent thromboembolism, stage 2 acute myocardial infarction, transfusion related acute lung injury.

Other non-limiting examples of diseases include disorders associated with function or development such as Acute Phase Response Signaling, LXR/RXR Activation, FXR/RXR Activation, Coagulation System, Complement System, Clathrin-mediated Endocytosis Signaling, Atherosclerosis Signaling, IL-12 Signaling and Production in Macrophages, Production of Nitric Oxide and Reactive Oxygen Species in Macrophages, Intrinsic Prothrombin Activation Pathway, Extrinsic Prothrombin Activation Pathway, Neuroprotective Role of THOP1 in Alzheimer's Disease, Hematopoiesis from Pluripotent Stem Cells, Primary Immunodeficiency Signaling, Systemic Lupus Erythematosus Signaling, Role of Pattern, Recognition Receptors in Recognition of Bacteria and Viruses, TR/RXR Activation, Role of Tissue Factor in Cancer, MSP-RON Signaling Pathway, Glioma Invasiveness Signaling, Caveolar-mediated Endocytosis Signaling, Communication between Innate and Adaptive Immune Cells and phagosome formation.

Other non-limiting examples of diseases include disorders associated with function or development such as cell movement, proliferation of cells, migration of cells, quantity of cells, organismal death, development of vasculature, transport of molecule, leukocyte migration, inflammatory response, activation of cells, morphology of body cavity, adhesion of blood cells, concentration of lipid, generation of cells, movement of myeloid cells, cell movement of phagocytes, fatty acid metabolism, synthesis of lipid, angiogenesis, quantity of blood cells, cellular homeostasis, aggregation of cells, metabolism of protein, aggregation of blood cells, adhesion of immune cells, binding of cells, metabolism of reactive oxygen species, activation of blood cells, vasculogenesis, abnormal morphology of body cavity, synthesis of reactive oxygen species, cell movement of neutrophils, quantity of metal, catabolism of protein, neurological signs, activation of leukocytes, coagulation, aggregation of blood platelets, quantity of leukocytes, coagulation of blood, Bleeding, quantity of steroid, immune response of cells, survival of organism, transport of lipid, synthesis of fatty acid, size of lesion, engulfment of cells, cellular infiltration, chemotaxis, binding of blood cells, activation of myeloid cells, activation of phagocytes, quantity of carbohydrate, ion homeostasis of cells, growth of epithelial tissue, transport of steroid, export of molecule, concentration of sterol, cellular infiltration by leukocytes, quantity of Ca2+, quantity of phagocytes, quantity of myeloid cells, secretion of molecule, morphology of connective tissue, cytolysis, flux of Ca2+, immune response of leukocytes, accumulation of cells, recruitment of cells, endothelial cell development, adhesion of myeloid cells, generation of reactive oxygen species, migration of phagocytes, concentration of fatty acid, proliferation of endothelial cells, activation of antigen presenting cells, concentration of triacylglycerol, recruitment of leukocytes, production of reactive oxygen species, quantity of protein in blood, Organ Degeneration, efflux of cholesterol, adhesion of phagocytes, synthesis of eicosanoid, response of myeloid cells, phagocytosis of cells, concentration of cholesterol, metabolism of membrane lipid derivative, morphology of blood cells, binding of phagocytes, permeability of vasculature, synthesis of prostaglandin, homeostasis of lipid, release of lipid, immune response of phagocytes, activation of macrophages, steroid metabolism, recruitment of phagocytes, accumulation of leukocytes, proteolysis, cell movement of macrophages, recruitment of myeloid cells, endocytosis by eukaryotic cells, quantity of granulocytes, complement activation, transport of phospholipid, size of atherosclerotic lesion, binding of endothelial cells, binding of professional phagocytic cells, cell movement of monocytes, hydrolysis of lipid, quantity of reactive oxygen species, secretion of lipid, influx of Ca2+, Shock Response, quantity of neutrophils, synthesis of nitric oxide, mass of organism, function of cardiovascular system, efflux of phospholipid, esterification of cholesterol, adhesion of blood platelets, quantity of protein lipid complex in blood, experimentally, induced diabetes, permeability of blood vessel, adhesion of granulocytes, metabolism of hydrogen, peroxide, binding of myeloid cells, metabolism of acylglycerol, uptake of lipid, engulfment of leukocytes, accumulation of myeloid cells, immune response of antigen presenting cells, recruitment of granulocytes, internalization of cells, metabolism of phospholipid, molecular cleavage of protein fragment, synthesis of steroid, area of atherosclerotic lesion, abortion, binding of blood platelets, quantity of hdl cholesterol in blood, adhesion of neutrophils, chemotaxis of monocytes, biosynthesis of hydrogen peroxide, metabolism of triacylglycerol, activation of blood platelets, metabolism of cholesterol, cell movement of epithelial cell lines, activation of neuroglia, accumulation of phagocytes, migration of granulocytes, phagocytosis of phagocytes, phagocytosis of myeloid cells, conversion of lipid, migration of muscle cells, metabolism of phosphatidic acid, migration of antigen presenting cells, healing of wound, size of infarct, proliferation of liver cells, recruitment of neutrophils, quantity of macrophages, habitual abortion, secretion of triacylglycerol, quantity of superoxide, binding of vascular endothelial cells, peroxidation of lipid, activation of endothelial cells, quantity of blood vessel, injury of cells, binding of lipid, binding of carbohydrate, injury of kidney, homeostasis of cholesterol, response of granulocytes, cell movement of embryonic cell lines, activation of granulocytes, adhesion of epithelial cells, immune response of T lymphocytes, phagocytosis of antigen presenting cells, quantity of muscle, synthesis of prostaglandin E2, formation of blood vessel, release of eicosanoid, transport of heavy metal, cell movement of leukemia cell lines, cell movement of kidney cell lines, receptor-mediated endocytosis, migration of smooth muscle cells, transmission of lipid, concentration of cholesterol ester, respiratory burst of granulocytes, adhesion of kidney cell lines, neovascularization of choroid, quantity of ldl, cholesterol in blood, relaxation of artery, recruitment of monocytes, function of blood platelets, synthesis of cyclic GMP, flow of blood, binding of polysaccharide, synthesis of cholesterol, binding of, antigen presenting cells, generation of superoxide, production of hydrogen peroxide, accumulation of macrophages, immune response of neutrophils, formation of reactive oxygen species, release of arachidonic acid, growth of bacteria, activation of neutrophils, quantity of heavy metal, damage of connective tissue, release of cholesterol, uptake of cholesterol ester, activation of carbohydrate, coagulation of plasma, quantity of lysophosphatidylcholine, metabolism of lipoprotein, internalization of lipid, movement of macrophage cancer cell lines, binding of cell surface, hydrolysis of triacylglycerol, respiratory burst of neutrophils, adhesion of embryonic cell lines, migration of eosinophils, quantity of endothelial cells, metabolism of phosphatidylcholine, binding of neutrophils, clearance of lipid, adhesion of epithelial cell lines, quantity of smooth muscle cells, adhesion of monocytes, recruitment of phospholipid, stimulation of endothelial tissue, surface area of atherosclerotic lesion, activation of inflammatory leukocytes, concentration of lipopolysaccharide, import of cholesterol, opsonization of cells, relaxation of coronary artery, synthesis of 12(S)-hydroxyeicosatetraenoic acid, lysis of blood clot, agglutination of blood cells, synthesis of 6-keto-prostaglandin F1 alpha, formation of plaque, release of phosphatidic acid, classical complement pathway, clearance of triacylglycerol, synthesis of corticosterone, activated partial thromboplastin time of plasma, auto-oxidation of amino acids, complement component C1s deficiency, delay in renal and urological disorder, density of artery, recruitment of phosphatidic acid.

The term “phenotype” as used herein comprises the composite of an organism's observable characteristics or traits, such as its morphology, development, biochemical or physiological properties, phenology, behavior, and products of behavior.

The term “diagnosis,” or “dx,” refers to the identification of the nature and cause of a certain phenomenon. As used herein, a diagnosis typically refers to a medical diagnosis, which is the process of determining which disease or condition explains a symptoms and signs. A diagnostic procedure, often a diagnostic test or assay, can be used to provide a diagnosis. A diagnosis can comprise detecting the presence of a disease or disorder. A diagnosis can comprise detecting the presence of a disease or disorder, or condition.

The term “prognosis,” or “px,” as used herein refers to predicting the likely outcome of a current standing. For example, a prognosis can include the expected duration and course of a disease or disorder, such as progressive decline or expected recovery.

The term “theranosis,” or “tx” as used herein refers to a diagnosis or prognosis used in the context of a medical treatment. For example, theranostics can include diagnostic testing used for selecting appropriate and optimal therapies (or the inverse) based on the context of genetic content or other molecular or cellular analysis. Theranostics includes pharmacogenomics, personalized and precision medicine.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy. Beneficial or desired clinical results include, but are not limited to, alleviation of one or more symptom(s), diminishment of extent of the deficit, stabilized (i.e., not worsening) state of progression, delay or slowing of progression or invasiveness, and amelioration or palliation of symptoms associated with the brain insulin resistance. Treatment also includes a decrease in mortality or an increase in the lifespan of a subject as compared to one not receiving the treatment.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder described herein. Treatment is generally “effective” if one or more symptoms are reduced. Alternatively, treatment is “effective” if the progression of a disease is reduced or halted. That is, “treatment” includes not just the improvement of symptoms, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment). As used herein, the terms “treat,” “treatment,” “treating,” or “amelioration” refer to therapeutic treatments and prophylactic or preventative measures, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder described herein. Treatment is generally “effective” if one or more symptoms are reduced. Alternatively, treatment is “effective” if the progression of a disease, disorder, or condition is reduced or halted. That is, “treatment” includes not just the improvement of symptoms, but also a cessation of at least slowing of progress or worsening of symptoms that would be expected in absence of treatment. Also, “treatment” may mean to pursue or obtain beneficial results, or lower the chances of the individual developing the condition, disease, or disorder even if the treatment is ultimately unsuccessful. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment). Those in need of treatment include those already with the condition, disease, or disorder as well as those prone to have the condition, disease, or disorder or those in whom the condition, disease, or disorder is to be prevented.

Non-limiting examples of treatments or therapeutic treatments include pharmacological or biological therapies and/or interventional surgical treatments.

The term “preventative treatment” means maintaining or improving a healthy state or non-diseased state of a healthy subject or subject that does not have a disease. The term “preventative treatment” or “health surveillance” also means to prevent or to slow the appearance of symptoms associated with a condition, disease, or disorder. The term “preventative treatment” also means to prevent or slow a subject from obtaining a condition, disease, or disorder.

As used herein, the term “administering,” refers to the placement of an agent as disclosed herein into a subject by a method or route which results in at least partial localization of the agents at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions can be in the form of aerosol, lotion, cream, gel, ointment, suspensions, solutions or emulsions. In accordance with the present invention, “administering” can be self-administering. For example, it is considered as “administering” that a subject consumes a composition as disclosed herein.

“Diagnostic” means identifying the presence or nature of a pathologic condition, disease, or disorder and includes identifying patients who are at risk of developing a specific condition, disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” While a particular diagnostic method may not provide a definitive diagnosis of a condition, a disease, or a disorder, it suffices if the method provides a positive indication that aids in diagnosis.

By “at risk of” is intended to mean at increased risk of, compared to a normal subject, or compared to a control group, e.g. a patient population. Thus a subject carrying a particular marker may have an increased risk for a specific condition, disease or disorder, and be identified as needing further testing. “Increased risk” or “elevated risk” mean any statistically significant increase in the probability, e.g., that the subject has the disorder. The risk is preferably increased by at least 10%, more preferably at least 20%, and even more preferably at least 50% over the control group with which the comparison is being made.

The term “statistically significant” or “significantly” refers to statistical evidence that there is a difference. It is defined as the probability of making a decision to reject the null hypothesis when the null hypothesis is actually true. The decision is often made using the p-value.

The terms “detection”, “detecting” and the like, may be used in the context of detecting biomarkers, detecting peptides, detecting proteins, or of detecting a condition, detecting a disease or a disorder.

The terms “proteases” and “peptidases” are used interchangeably herein to mean enzymes that breakdown proteins and peptides.

The terms “marker” or “biomarker” are used interchangeably herein, and in the context of the present invention refer to a protein or peptide (for example, protein or peptide associated with a disease, function, pathological process, or physiological process) is differentially present in a sample taken from patients having a specific disease or disorder as compared to a control value, the control value consisting of, for example average or mean values in comparable samples taken from control subjects (e.g., a person with a negative diagnosis, normal or healthy subject). Biomarkers may be determined as specific peptides or proteins which may be detected by mass spectroscopy. In some applications, for example, a mass spectroscopy may be used to determine multiple biomarkers, and differences between individual biomarkers and/or the partial or complete profile may be used for diagnosis.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of a particular disease or disorder or function or pathological process or physiological process. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person who does not suffer from the disease or disorder or function or pathological process or physiological process sought to be diagnosed. A control amount can be either in absolute amount (e.g., μg/ml) or a relative amount (e.g., relative intensity of signals).

The term “differentially present” or “change in level” refers to differences in the quantity and/or the frequency of a marker present in a sample taken from patients having a specific disease or disorder or function or pathological process or physiological process as compared to a control subject. For example, a marker can be present at an elevated level or at a decreased level in samples of patients with the disease or disorder or pathological process or physiological process compared to a control value (e.g. determined from samples of control subjects). Alternatively, a marker can be detected at a higher frequency or at a lower frequency in samples of patients compared to samples of control subjects.

A marker, compound, composition or substance is differentially present in a sample if the amount of the marker, compound, composition or substance in the sample is statistically significantly different from the amount of the marker, compound, composition or substance in another sample, or from a control value. For example, a compound is differentially present if it is present at least about 120%, at least about 130%, at least about 150%, at least about 180%, at least about 200%, at least about 300%, at least about 500%, at least about 700%, at least about 900%, or at least about 1000% greater or less than it is present in the other sample (e.g. control), or if it is detectable in one sample and not detectable in the other.

Alternatively or additionally, a marker, compound, composition or substance is differentially present between samples if the frequency of detecting the marker, etc. in samples of patients suffering from a particular disease or disorder or function or pathological process or physiological process, is statistically significantly higher or lower than in the control samples or control values obtained from healthy individuals. For example, a biomarker is differentially present between the two sets of samples if it is detected at least about 10%, at least about 20%, at least about 30%, at least about 40%, at least about 50%, at least about 60%, at least about 70%, at least about 80%, at least about 90%, or at least about 100% more frequently or less frequently observed in one set of samples than the other set of samples. These exemplary values notwithstanding, it is expected that a skilled practitioner can determine cut-off points, etc. that represent a statistically significant difference to determine whether the marker is differentially present.

General molecular biology terminology and techniques are known to those of skill in the art. See, e.g., Sambrook et al., Molecular Cloning: A Laboratory Manual, Cold Spring Harbor Press, N.Y., (3.sup.rd ed., 2000); and Brent et al., Current Protocols in Molecular Biology, John Wiley & Sons, Inc. (ringbou ed., 2003).

Abbreviations: MS, Mass Spectrometry; LC-MS/MS, liquid chromatography-tandem mass spectrometry; LC-SRM-MS, liquid chromatography selected reaction monitoring mass spectrometry; OGS, N-octyl glucoside; MMTS, Methyl methanethiosulfonate; TCEP, Tris-(2-carboxyethyl)-phosphine; FA, Formic acid; TPCK treated trypsin, Trypsin treated with L-(tosylamido-2-phenyl) ethyl chloromethyl ketone; β-Gal, β-galactosidase; CV %. Coefficient of variation; LC-SRM-MS, Liquid chromatography-selected reaction monitoring mass spectrometry; SIL peptide, Stable Isotope-Labeled Peptide; DIA-MS, Data Independent acquisition mass spectrometry; SPE, solid phase extraction; IQ panel, internal quality panel.

Phenotypes

As described herein, the compositions and methods of the invention may be used to characterize a phenotype in a sample of interest. The phenotype can be any phenotype of interest that may be characterized using the subject compositions and methods. Consider a non-limiting example wherein the phenotype comprises a disease or disorder. In such cases, the characterizing may be providing a diagnosis, prognosis or theranosis for the disease or disorder. In an illustrative embodiment, a sample from a subject is analyzed using the compositions and methods of the invention. The analysis is then used to predict or determine the presence, stage, grade, outcome, or likely therapeutic response of a disease or disorder in the subject. The analysis can also be used to assist in making such prediction or determination. As described herein, the compositions, methods, protein panels, articles of manufacture, or systems of the invention may be used to characterize a phenotype in a sample of interest. The phenotype can be any phenotype of interest that may be characterized using the subject compositions, methods, protein panels, articles of manufacture, or systems. Consider a non-limiting example wherein the phenotype comprises a disease or disorder. In such cases, the characterizing may be providing a diagnosis, prognosis or theranosis for the disease or disorder. In an illustrative embodiment, a sample from a subject is analyzed using the compositions, methods, protein panels, articles of manufacture, or systems of the invention. The analysis is then used to predict or determine the presence, stage, grade, outcome, or likely therapeutic response of a disease or disorder in the subject. The analysis can also be used to assist in making such prediction or determination.

In various embodiments the invention provides a method to identify protein biomarkers and patterns that are indicative a disease is or may be present. In some embodiments these methods may provide objective rationale for further testing. In various embodiments the invention provides a method for the identification of a plurality of proteins from a sample, wherein each protein is correlated to one or more peptides, wherein each peptide is correlated to one or more transitions, wherein each transition comprises a Q1 mass value and a Q3 mass value. In various embodiments the invention provides a method for the identification of a plurality of proteins from a sample, wherein each protein is correlated to one or more peptides, wherein each peptide is correlated to one or more transitions, wherein each transition comprises a Q1/Q3 mass value pair.

As used herein, SRM stands for selected reaction monitoring. As used herein, MRM stands for multiple reaction monitoring. As used herein, SWATH stands for sequential window acquisition of all theoretical fragment ion spectra. As used herein, DIA stands for data-independent analysis. As used herein, MS stands for mass spectrometry. As used herein, SIL stands for stable isotope-labeled. As used herein, DDA stands for data-dependent analysis. As used herein, PRM stands for parallel reaction monitoring.

As used herein, “MS data” can be raw MS data obtained from a mass spectrometer and/or processed MS data in which peptides and their fragments (e.g., transitions and MS peaks) are already identified, analyzed and/or quantified. MS data can be Selective Reaction Monitoring (SRM) data, Multiple Reaction Monitoring (MRM) data, Shotgun CID MS data, Original DIA MS Data, MSE MS data, p2CID MS Data, PAcIFIC MS Data, AIF MS Data, XDLA MS Data, SWATH MS data, or FT-ARM MS Data, or their combinations.

This approach of the present invention, based on SRM and/or SWATH MS, allows for the detection and accurate quantification of specific peptides in complex mixtures.

Selected Reaction Monitoring or Multiple Reaction Monitoring (SRM/MRM) mass spectrometry is a technology with the potential for reliable and comprehensive quantification of substances of low abundance in complex samples. SRM is performed on triple quadrupole-like instruments, in which increased selectivity is obtained through collision-induced dissociation. It is a non-scanning mass spectrometry technique, where two mass analyzers (Q1 and Q3) are used as static mass filters, to monitor a particular fragment of a selected precursor. On triple quadrapole instruments, various ionization methods can be used including without limitation electrospray ionization, chemical ionization, electron ionization, atmospheric pressure chemical ionization, and matrix-assisted laser desorption ionization. Both the first mass analyzer and the collision cell are continuously exposed to ions from the source in a time dependent manner. Once the ions move into the third mass analyzer time dependence becomes a factor. On triple quadrupole instruments, the first quadrapole mass filter, Q1, is the primary m/z selector after the sample leaves the ionization source. Any ions with mass-to-charge ratios other than the one selected for will not be allowed to infiltrate Q1. The collision cell, denoted as “q2”, located between the first quadrapole mass filter Q1 and second quadrapole mass filter Q3, is where fragmentation of the sample occurs in the presence of an inert gas like argon, helium, or nitrogen. Upon exiting the collision cell, the fragmented ions then travel onto the second quadrapole mass filter Q3, where m/z selection can occur again. The specific pair of mass-over-charge (m/z) values associated to the precursor and fragment ions selected is referred to as a “transition”. The detector acts as a counting device for the ions matching the selected transition thereby returning an intensity distribution over time. MRM is when multiple SRM transitions are measured within the same experiment on the chromatographic time scale by rapidly switching between the different precursor/fragment pairs. Typically, the triple quadrupole instrument cycles through a series of transitions and records the signal of each transition in relation to the elution time. The method allows for additional selectivity by monitoring the chromatographic co-elution of multiple transitions for a given analyte.

SWATH MS a data independent acquisition (DIA) method which aims to complement traditional mass spectrometry-based proteomics techniques such as shotgun and SRM methods. In essence, it allows a complete and permanent recording of all fragment ions of the detectable peptide precursors present in a biological sample. It thus combines the advantages of shotgun (high throughput) with those of SRM (high reproducibility and consistency).

In a preferred embodiment, the developed methods herein can be applied to the quantification of polypeptides(s) in biological sample(s). Any kind of biological samples comprising polypeptides can be the starting point and be analyzed by the methods herein. Indeed, any protein/peptide containing sample can be used for and analyzed by the methods produced here (cells, tissues, body fluids, waters, food, terrain, synthetic preparations, etc.). The methods herein can also be used with peptide mixtures obtained by digestion. Digestion of a polypeptide includes any kind of cleavage strategies, such as, enzymatic, chemical, physical or combinations thereof.

The deciding factors of which polypeptide will be the one of interest varies. It can be decided by performing a literature search and identifying proteins that are functionally related, are candidate protein biomarkers which can be used in screening for drug discovery, biomarker discovery and/or disease clinical phase trials or are diagnostic markers to screen for pharmaceutical/medical purposes. The polypeptide of interest may be determined by experimental analysis.

According to some embodiments, the following parameters of the methods provided herein are determined: trypsin digestion and peptide clean up, best responding polypeptides, best responding fragments, fragment intensity ratios (increased high and reproducible peak intensities), optimal collision energies, and all the optimal parameters to maximize sensitivity and/or specificity of the methods.

In other embodiments, quantification of the polypeptides and/or of the corresponding proteins or activity/regulation of the corresponding proteins is desired. A selected peptide is labeled with a stable-isotope and used as an internal standard to achieve absolute quantification of a protein of interest. The addition of a quantified stable-labeled peptide analogue of the tag to the peptide sample in known amount; and subsequently the tag and the peptide of interest is quantified by mass spectrometry and absolute quantification of the endogenous levels of the proteins is obtained.

According to a preferred embodiment, the analysis and/or comparison is done on protein samples of wild-type or physiological/healthy origin with protein samples of mutant or pathological origin.

The present invention supports the use of SRM and SWATH as platform to identify signature polypeptides for quantitative proteomics. The approach is applicable to the analysis of proteins from all organisms, from cells, organs, body fluids, and in the context of in vivo and/or in vitro analyses. Examples of applications of the invention include the development, use and commercialization of quantitative assays for sets of polypeptides of interest. The invention can be beneficial for the pharmaceutical industry (e.g. drug development and assessment), the biotechnology industry (e.g. assay design and development and quality control), and in clinical applications (e.g. identification of biomarkers of disease and quantitative analysis for diagnostic, prognostic and/or therapeutic use). The invention can also be applied to water, drink, food and food ingredient testing, for example, quantifying nutrients, contaminants, toxins, antibiotics, steroids, hormones, pathogens, and allergens in water, drinks, foods and food ingredients.

Methods of the Invention

Various embodiments of the present invention provide a method of obtaining a biomarker signature for a subject, the method comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1 mass value and a Q3 mass value according to Table 5. Various embodiments of the present invention provide a method of obtaining a biomarker signature for a subject, the method comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1/Q3 mass value pair according to Table 5. Various embodiments of the present invention provide a method of obtaining a biomarker signature for a subject, the method comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is one or more Q1/Q3 mass value pairs according to Table 5. Various embodiments of the present invention provide a method of obtaining a biomarker signature for a subject, the method comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is two or more Q1/Q3 mass value pairs according to Table 5. Various embodiments of the present invention provide a method of obtaining a biomarker signature for a subject, the method comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1 mass value according to Table 5. Various embodiments of the present invention provide a method of obtaining a biomarker signature for a subject, the method comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is one or more Q1 mass values according to Table 5. Various embodiments of the present invention provide a method of obtaining a biomarker signature for a subject, the method comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is two or more Q1 mass values according to Table 5.

Various embodiments of the present invention provide a method of obtaining a biomarker signature for a subject, the method comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1 mass value and a Q3 mass value according to Table 5. In some embodiments, the Q1 mass value and the Q3 mass value are correlated to the one or more peptides according to Table 5. In some embodiments, the one or more peptides are correlated to one or more proteins according to Table 5. In some embodiments, the one or more biomarkers are the one or more proteins according to Table 5. In some embodiments, the mass spectrometry technique is selected reaction monitoring (SRM) or multiple reaction monitoring (MRM). In some embodiments, the mass spectrometry technique is liquid chromatography-selected reaction monitoring-mass spectrometry (LC-SRM-MS). In some embodiments, the mass spectrometry technique is data-independent acquisition mass spectrometry (DIA MS). In some embodiments, the method further comprises comparing the biomarker signature from the subject to a biomarker signature from a reference sample. In some embodiments, the method further comprises making an assessment of the subject based on the comparison, wherein the assessment is a diagnosis of a disease. In some embodiments, the mass spectrometer is a triple quadrupole mass spectrometer. In some embodiments, the reference sample is obtained from a control subject, wherein the control subject does not have the disease. In some embodiments, the reference sample is obtained from the subject before the subject is treated for the disease. In some embodiments, the reference sample is from a subject that has been successfully treated for the disease.

Various embodiments of the present invention provide a method of obtaining a biomarker signature for a subject, the method comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1/Q3 mass value pair according to Table 5. In some embodiments, the Q1/Q3 mass value pairs are correlated to the one or more peptides according to Table 5. In some embodiments, the one or more peptides are correlated to one or more proteins according to Table 5. In some embodiments, the one or more biomarkers are the one or more proteins according to Table 5. In some embodiments, the Q1/Q3 mass value pair is one or more Q1/Q3 mass value pairs. In some embodiments, the Q1/Q3 mass value pair is two or more Q1/Q3 mass value pairs. In some embodiments, the mass spectrometry technique is selected reaction monitoring (SRM) or multiple reaction monitoring (MRM). In some embodiments, the mass spectrometry technique is liquid chromatography-selected reaction monitoring-mass spectrometry (LC-SRM-MS). In some embodiments, the mass spectrometry technique is data-independent acquisition mass spectrometry (DIA MS). In some embodiments, the method further comprises comparing the biomarker signature from the subject to a biomarker signature from a reference sample. In some embodiments, the method further comprises making an assessment of the subject based on the comparison, wherein the assessment is a diagnosis of a disease. In some embodiments, the mass spectrometer is a triple quadrupole mass spectrometer. In some embodiments, the reference sample is obtained from a control subject, wherein the control subject does not have the disease. In some embodiments, the reference sample is obtained from the subject before the subject is treated for the disease. In some embodiments, the reference sample is from a subject that has been successfully treated for the disease.

Various embodiments of the invention provide method of treating a subject for a disease, the method comprising: (a) making an assessment of the subject based on the biomarker signature, wherein the assessment is a diagnosis of the disease; and (b) treating the subject based on the assessment, wherein the biomarker signature is obtained by obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1 mass value and a Q3 mass value according to Table 5.

Various embodiments of the invention provide method of treating a subject for a disease, the method comprising: (a) making an assessment of the subject based on the biomarker signature, wherein the assessment is a diagnosis of the disease; and (b) treating the subject based on the assessment, wherein the biomarker signature is obtained by obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1/Q3 mass value pair according to Table 5. In some embodiments, the Q1/Q3 mass value pair is one or more Q1/Q3 mass value pairs. In some embodiments, the Q1/Q3 mass value pair is two or more Q1/Q3 mass value pairs.

Various embodiments of the invention provide method of treating a subject for a disease, the method comprising: (a) making an assessment of the subject based on the biomarker signature, wherein the assessment is a diagnosis of the disease; and (b) treating the subject based on the assessment, wherein the biomarker signature is obtained by obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1 mass value according to Table 5. In some embodiments, the Q1 mass value is one or more Q1 mass values. In some embodiments, the Q1 mass value is two or more Q1 mass values.

Various embodiments of the present invention provide a method of identifying a plurality of biomarkers in a sample, comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1 mass value and a Q3 mass value according to Table 5.

Various embodiments of the present invention provide a method of identifying a plurality of biomarkers in a sample, comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1/Q3 mass value pair according to Table 5. In some embodiments, the Q1/Q3 mass value pair is one or more Q1/Q3 mass value pairs. In some embodiments, the Q1/Q3 mass value pair is two or more Q1/Q3 mass value pairs.

Various embodiments of the present invention provide a method of identifying a plurality of biomarkers in a sample, comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1 mass value according to Table 5. In some embodiments, the Q1 mass value is one or more Q1 mass values. In some embodiments, the Q1 mass value is two or more Q1 mass values.

Various embodiments of the present invention provide a method of early screening for a plurality of biomarkers in a sample, comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1 mass value and a Q3 mass value according to Table 5.

Various embodiments of the present invention provide a method of early screening for a plurality of biomarkers in a sample, comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1/Q3 mass value pair according to Table 5. In some embodiments, the Q1/Q3 mass value pair is one or more Q1/Q3 mass value pairs. In some embodiments, the Q1/Q3 mass value pair is two or more Q1/Q3 mass value pairs.

Various embodiments of the present invention provide a method of early screening for a plurality of biomarkers in a sample, comprising: obtaining a sample from the subject; treating the sample with a protease to obtain a digested sample; measuring in the digested sample a representation of one or more biomarkers to obtain the biomarker signature for the subject, wherein the measuring is performed using a mass spectrometer and a mass spectrometry technique, and the representation of the one or more biomarkers is a Q1 mass value according to Table 5. In some embodiments, the Q1 mass value is one or more Q1 mass values. In some embodiments, the Q1 mass value is two or more Q1 mass values.

Various embodiments of the present invention provide a method of diagnosing a disease in a subject, comprising: treating the sample with a protease to obtain a digested sample; measuring the amount of at least one biomarker in the digested sample using a mass spectrometer and a mass spectrometry technique, wherein the at least one biomarker is one or more peptides; and comparing the amount of at least one biomarker measured in the digested sample to a reference amount in a normal subject population, wherein a change in the amount, compared to the reference amount, is indicative of the disease. In some embodiments the mass spectrometer is a triple quadrupole mass spectrometer. In some embodiments, the triple quadrupole mass spectrometer comprises a quadrupole mass filter Q1 and a quadrupole mass filter Q3, wherein the quadrupole mass filter Q1 has a Q1 mass value and the quadrupole mass filter Q3 has a Q3 mass value. In some embodiments, the Q1 mass value and the Q3 mass value are correlated to the one or more peptides according to Table 5. In some embodiments, the one or more peptides are correlated to one or more proteins according to Table 5. In some embodiments, the mass spectrometry technique is selected reaction monitoring (SRM) or multiple reaction monitoring (MRM). In some embodiments, the mass spectrometry technique is liquid chromatography-selected reaction monitoring-mass spectrometry (LC-SRM-MS). In some embodiments, the mass spectrometry technique is data-independent acquisition mass spectrometry (DIA MS). In some embodiments, the change is an increase or decrease in the amount of at least one biomarker compared to the reference amount.

Various embodiments of the present invention provide a method of obtaining a biomarker signature for a subject, the method comprising: obtaining a sample from the subject; treating the sample with a protease to obtain a digested sample; and measuring in the digested sample a representation of one or more biomarkers to obtain the biomarker signature for the subject, wherein the measuring is performed using a mass spectrometer and a mass spectrometry technique, and the representation of the one or more biomarkers is obtained using a mass spectrometry assay, wherein the mass spectrometry assay comprises a quantitation of a Q1/Q3 mass value pair according to Table 5, wherein the Q1/Q3 mass value pair is used to quantify a corresponding peptide and protein according to Table 5. In some embodiments, the Q1/Q3 mass value pair is one or more the Q1/Q3 mass value pairs. In some embodiments, the Q1/Q3 mass value pair is two or more the Q1/Q3 mass value pairs. In some embodiments, the protease is trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof. In some embodiments, the protease is trypsin. In some embodiments, the mass spectrometer is a triple quadrupole mass spectrometer. In some embodiments, each Q1/Q3 mass value pair are correlated to the one or more peptides according to Table 5. In some embodiments, the one or more peptides are correlated to one or more proteins according to Table 5. In some embodiments, the one or more biomarkers are the one or more proteins according to Table 5. In some embodiments, the mass spectrometry technique is selected reaction monitoring (SRM) or multiple reaction monitoring (MRM). In some embodiments, the mass spectrometry technique is liquid chromatography-selected reaction monitoring-mass spectrometry (LC-SRM-MS). In some embodiments, the mass spectrometry technique is data-independent acquisition mass spectrometry (DIA MS). In some embodiments, the method further comprises adding a stable-isotope labeled peptide standard to the sample. In some embodiments, the method further comprises comparing the biomarker signature from the subject to a biomarker signature from a reference sample. In some embodiments, the method further comprises making an assessment of the subject based on the comparison, wherein the assessment is a diagnosis of a disease. In some embodiments, the sample is plasma, serum, cerebrospinal fluid (CSF), a tissue extract, or a biopsy sample. In some embodiments, the reference sample is obtained from a control subject, wherein the control subject does not have the disease. In some embodiments, the reference sample is obtained from the subject before the subject is treated for the disease. In some embodiments, the reference sample is from a subject that has been successfully treated for the disease.

Various embodiments of the present invention provide a method of treating a subject for a disease, the method comprising: (a) making an assessment of the subject based on a biomarker signature for the subject, wherein the assessment is a diagnosis of the disease; and (b) treating the subject based on the assessment, wherein the biomarker signature for the subject is obtained by a method comprising, obtaining a sample from the subject; treating the sample with a protease to obtain a digested sample; and measuring in the digested sample a representation of one or more biomarkers to obtain the biomarker signature for the subject, wherein the measuring is performed using a mass spectrometer and a mass spectrometry technique, and the representation of the one or more biomarkers is obtained using a mass spectrometry assay, wherein the mass spectrometry assay comprises a quantitation of a Q1/Q3 mass value pair according to Table 5, wherein the Q1/Q3 mass value pair is used to quantify a corresponding peptide and protein according to Table 5. In some embodiments, the Q1/Q3 mass value pair is one or more the Q1/Q3 mass value pairs. In some embodiments, the Q1/Q3 mass value pair is two or more the Q1/Q3 mass value pairs.

Various embodiments of the present invention provide a method of obtaining a biomarker signature for a subject, the method comprising: obtaining a sample from the subject; treating the sample with a protease to obtain a digested sample; and measuring in the digested sample a representation of one or more biomarkers to obtain the biomarker signature for the subject, wherein the measuring is performed using a mass spectrometer and a mass spectrometry technique, and the representation of the one or more biomarkers is obtained using a mass spectrometry assay, wherein the mass spectrometry assay comprises a quantitation of a Q1 mass value according to Table 5, wherein the Q1 mass value is used to quantify a corresponding peptide and protein according to Table 5. In some embodiments, the Q1 mass value is one or more Q1 mass values. In some embodiments, the Q1 mass value is two or more Q1 mass values. In some embodiments, the protease is trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof. In some embodiments, the protease is trypsin. In some the mass spectrometer is a quadrupole time-of-flight (QTOF) mass spectrometer or a hybrid quadrupole-orbitrap (QOrbitrap) mass spectrometer. In some embodiments, each Q1 mass value is correlated to the one or more peptides according to Table 5. In some embodiments, the one or more peptides are correlated to one or more proteins according to Table 5. In some embodiments, the one or more biomarkers are the one or more proteins according to Table 5. In some embodiments, the mass spectrometry technique is parallel reaction monitoring (PRM). In some embodiments, the mass spectrometry technique is liquid chromatography-parallel reaction monitoring-mass spectrometry (LC-PRM-MS). In some embodiments, the method further comprises adding a stable-isotope labeled peptide standard to the sample. In some embodiments, the method further comprises comparing the biomarker signature from the subject to a biomarker signature from a reference sample. In some embodiments, the method further comprises making an assessment of the subject based on the comparison, wherein the assessment is a diagnosis of a disease. In some embodiments, the sample is plasma, serum, cerebrospinal fluid (CSF), a tissue extract, or a biopsy sample. In some embodiments, the reference sample is obtained from a control subject, wherein the control subject does not have the disease. In some embodiments, the reference sample is obtained from the subject before the subject is treated for the disease. In some embodiments, the reference sample is from a subject that has been successfully treated for the disease.

Various embodiments of the present invention provide a method of treating a subject for a disease, the method comprising: (a) making an assessment of the subject based on a biomarker signature for the subject, wherein the assessment is a diagnosis of the disease; and (b) treating the subject based on the assessment, wherein the biomarker signature for the subject is obtained by a method comprising: obtaining a sample from the subject; treating the sample with a protease to obtain a digested sample; and measuring in the digested sample a representation of one or more biomarkers to obtain the biomarker signature for the subject, wherein the measuring is performed using a mass spectrometer and a mass spectrometry technique, and the representation of the one or more biomarkers is obtained using a mass spectrometry assay, wherein the mass spectrometry assay comprises a quantitation of a Q1 mass value according to Table 5, wherein the Q1 mass value is used to quantify a corresponding peptide and protein according to Table 5. In some embodiments, the Q1 mass value is one or more Q1 mass values. In some embodiments, the Q1 mass value is two or more Q1 mass values.

Various embodiments of the present invention provide a method for assessing the efficacy of a treatment, comprising: comparing a biomarker signature from a subject to a biomarker signature from a reference sample, wherein a change in the biomarker signature from the subject relative to the biomarker signature from the reference sample is indicative of the efficacy of the treatment, wherein the treatment is according to a method of treating a subject for a disease, the method comprising: (a) making an assessment of the subject based on the biomarker signature, wherein the assessment is a diagnosis of the disease; and (b) treating the subject based on the assessment, wherein the biomarker signature for the subject is obtained by a method comprising, obtaining a sample from the subject; treating the sample with a protease to obtain a digested sample; and measuring in the digested sample a representation of one or more biomarkers to obtain the biomarker signature for the subject, wherein the measuring is performed using a mass spectrometer and a mass spectrometry technique, and the representation of the one or more biomarkers is obtained using a mass spectrometry assay, wherein the mass spectrometry assay comprises a quantitation of a Q1/Q3 mass value pair according to Table 5, wherein the Q1/Q3 mass value pair is used to quantify a corresponding peptide and protein according to Table 5. In some embodiments, the Q1/Q3 mass value pair is one or more the Q1/Q3 mass value pairs. In some embodiments, the Q1/Q3 mass value pair is two or more the Q1/Q3 mass value pairs. In some embodiments, the reference sample is obtained from a control subject, wherein the control subject does not have the disease. In some embodiments, the reference sample is obtained from the subject before the subject is treated for the disease. In some embodiments, the reference sample is from a subject that has been successfully treated for the disease.

Various embodiments of the present invention provide a method for assessing the efficacy of a treatment, comprising: comparing a biomarker signature from a subject to a biomarker signature from a reference sample, wherein a change in the biomarker signature from the subject relative to the biomarker signature from the reference sample is indicative of the efficacy of the treatment, wherein the treatment is according to a method of treating a subject for a disease, the method comprising: (a) making an assessment of the subject based on the biomarker signature, wherein the assessment is a diagnosis of the disease; and (b) treating the subject based on the assessment, wherein the biomarker signature for the subject is obtained by a method comprising: obtaining a sample from the subject; treating the sample with a protease to obtain a digested sample; and measuring in the digested sample a representation of one or more biomarkers to obtain the biomarker signature for the subject, wherein the measuring is performed using a mass spectrometer and a mass spectrometry technique, and the representation of the one or more biomarkers is obtained using a mass spectrometry assay, wherein the mass spectrometry assay comprises a quantitation of a Q1 mass value according to Table 5, wherein the Q1 mass value is used to quantify a corresponding peptide and protein according to Table 5. In some embodiments, the Q1 mass value is one or more Q1 mass values. In some embodiments, the Q1 mass value is two or more Q1 mass values. In some embodiments, the reference sample is obtained from a control subject, wherein the control subject does not have the disease. In some embodiments, the reference sample is obtained from the subject before the subject is treated for the disease. In some embodiments, the reference sample is from a subject that has been successfully treated for the disease.

Functions

As used herein, the term “function” is in reference to an organism, organ, a biological process, a biological system, a cellular process, a cellular system, a molecular process, a molecular system and the like, non-limiting examples of which include lipid metabolism, redox signaling, immune response, hematological system development, hematological system function, reproductive system development, reproductive system function, and gene expression. In some embodiments, the function is a physiological process.

As used herein, the term “function” is in reference to an organism, organ, a biological process, a biological system, a cellular process, a cellular system, a molecular process, a molecular system and the like, non-limiting examples of which include cellular movement, cellular growth, cell proliferation, tissue morphology, organismal survival, molecular transport, immune cell trafficking, cell-to-cell signaling, cell-to-cell interaction, lipid metabolism, small molecule biochemistry, tissue development, protein synthesis, free radical scavenging, protein degradation, protein synthesis, hematological system development, hematological system function, tissue morphology, carbohydrate metabolism, cellular function and maintenance, cell signaling, vitamin metabolism, mineral metabolism, digestive system development, digestive system function, hepatic system development, hepatic system function, hair development, hair function, skin development, skin function, nervous system development, nervous system function, cellular compromise, nucleic acid metabolism, small molecule biochemistry, organ morphology, organismal development, renal system development, renal system function, urological system development, urological system function, humoral immune response, amino acid metabolism, energy production, post-translational modification. In some embodiments, the function is a physiological process.

As used herein, the term “function” is in reference to an organism, organ, a biological process, a biological system, a cellular process, a cellular system, a molecular process, a molecular system and the like, non-limiting examples of which include Acute Phase Response Signaling, LXR/RXR Activation, FXR/RXR Activation, Coagulation System, Complement System, Clathrin-mediated Endocytosis Signaling, Atherosclerosis Signaling, IL-12 Signaling and Production in Macrophages, Production of Nitric Oxide and Reactive Oxygen Species in Macrophages, Intrinsic Prothrombin Activation Pathway, Extrinsic Prothrombin Activation Pathway, Neuroprotective Role of THOP1 in Alzheimer's Disease, Hematopoiesis from Pluripotent Stem Cells, Primary Immunodeficiency Signaling, Systemic Lupus Erythematosus Signaling, Role of Pattern, Recognition Receptors in Recognition of Bacteria and Viruses, TR/RXR Activation, Role of Tissue Factor in Cancer, MSP-RON Signaling Pathway, Glioma Invasiveness Signaling, Caveolar-mediated Endocytosis Signaling, Communication between Innate and Adaptive Immune Cells and phagosome formation. In some embodiments, the function is a physiological process.

As used herein, the term “function” is in reference to an organism, organ, a biological process, a biological system, a cellular process, a cellular system, a molecular process, a molecular system and the like, non-limiting examples of which include cell movement, proliferation of cells, migration of cells, quantity of cells, organismal death, development of vasculature, transport of molecule, leukocyte migration, inflammatory response, activation of cells, morphology of body cavity, adhesion of blood cells, concentration of lipid, generation of cells, movement of myeloid cells, cell movement of phagocytes, fatty acid metabolism, synthesis of lipid, angiogenesis, quantity of blood cells, cellular homeostasis, aggregation of cells, metabolism of protein, aggregation of blood cells, adhesion of immune cells, binding of cells, metabolism of reactive oxygen species, activation of blood cells, vasculogenesis, abnormal morphology of body cavity, synthesis of reactive oxygen species, cell movement of neutrophils, quantity of metal, catabolism of protein, neurological signs, activation of leukocytes, coagulation, aggregation of blood platelets, quantity of leukocytes, coagulation of blood, Bleeding, quantity of steroid, immune response of cells, survival of organism, transport of lipid, synthesis of fatty acid, size of lesion, engulfment of cells, cellular infiltration, chemotaxis, binding of blood cells, activation of myeloid cells, activation of phagocytes, quantity of carbohydrate, ion homeostasis of cells, growth of epithelial tissue, transport of steroid, export of molecule, concentration of sterol, cellular infiltration by leukocytes, quantity of Ca2+, quantity of phagocytes, quantity of myeloid cells, secretion of molecule, morphology of connective tissue, cytolysis, flux of Ca2+, immune response of leukocytes, accumulation of cells, recruitment of cells, endothelial cell development, adhesion of myeloid cells, generation of reactive oxygen species, migration of phagocytes, concentration of fatty acid, proliferation of endothelial cells, activation of antigen presenting cells, concentration of triacylglycerol, recruitment of leukocytes, production of reactive oxygen species, quantity of protein in blood, Organ Degeneration, efflux of cholesterol, adhesion of phagocytes, synthesis of eicosanoid, response of myeloid cells, phagocytosis of cells, concentration of cholesterol, metabolism of membrane lipid derivative, morphology of blood cells, binding of phagocytes, permeability of vasculature, synthesis of prostaglandin, homeostasis of lipid, release of lipid, immune response of phagocytes, activation of macrophages, steroid metabolism, recruitment of phagocytes, accumulation of leukocytes, proteolysis, cell movement of macrophages, recruitment of myeloid cells, endocytosis by eukaryotic cells, quantity of granulocytes, complement activation, transport of phospholipid, size of atherosclerotic lesion, binding of endothelial cells, binding of professional phagocytic cells, cell movement of monocytes, hydrolysis of lipid, quantity of reactive oxygen species, secretion of lipid, influx of Ca2+, Shock Response, quantity of neutrophils, synthesis of nitric oxide, mass of organism, function of cardiovascular system, efflux of phospholipid, esterification of cholesterol, adhesion of blood platelets, quantity of protein lipid complex in blood, experimentally, induced diabetes, permeability of blood vessel, adhesion of granulocytes, metabolism of hydrogen, peroxide, binding of myeloid cells, metabolism of acylglycerol, uptake of lipid, engulfment of leukocytes, accumulation of myeloid cells, immune response of antigen presenting cells, recruitment of granulocytes, internalization of cells, metabolism of phospholipid, molecular cleavage of protein fragment, synthesis of steroid, area of atherosclerotic lesion, abortion, binding of blood platelets, quantity of hdl cholesterol in blood, adhesion of neutrophils, chemotaxis of monocytes, biosynthesis of hydrogen peroxide, metabolism of triacylglycerol, activation of blood platelets, metabolism of cholesterol, cell movement of epithelial cell lines, activation of neuroglia, accumulation of phagocytes, migration of granulocytes, phagocytosis of phagocytes, phagocytosis of myeloid cells, conversion of lipid, migration of muscle cells, metabolism of phosphatidic acid, migration of antigen presenting cells, healing of wound, size of infarct, proliferation of liver cells, recruitment of neutrophils, quantity of macrophages, habitual abortion, secretion of triacylglycerol, quantity of superoxide, binding of vascular endothelial cells, peroxidation of lipid, activation of endothelial cells, quantity of blood vessel, injury of cells, binding of lipid, binding of carbohydrate, injury of kidney, homeostasis of cholesterol, response of granulocytes, cell movement of embryonic cell lines, activation of granulocytes, adhesion of epithelial cells, immune response of T lymphocytes, phagocytosis of antigen presenting cells, quantity of muscle, synthesis of prostaglandin E2, formation of blood vessel, release of eicosanoid, transport of heavy metal, cell movement of leukemia cell lines, cell movement of kidney cell lines, receptor-mediated endocytosis, migration of smooth muscle cells, transmission of lipid, concentration of cholesterol ester, respiratory burst of granulocytes, adhesion of kidney cell lines, neovascularization of choroid, quantity of ldl, cholesterol in blood, relaxation of artery, recruitment of monocytes, function of blood platelets, synthesis of cyclic GMP, flow of blood, binding of polysaccharide, synthesis of cholesterol, binding of, antigen presenting cells, generation of superoxide, production of hydrogen peroxide, accumulation of macrophages, immune response of neutrophils, formation of reactive oxygen species, release of arachidonic acid, growth of bacteria, activation of neutrophils, quantity of heavy metal, damage of connective tissue, release of cholesterol, uptake of cholesterol ester, activation of carbohydrate, coagulation of plasma, quantity of lysophosphatidylcholine, metabolism of lipoprotein, internalization of lipid, movement of macrophage cancer cell lines, binding of cell surface, hydrolysis of triacylglycerol, respiratory burst of neutrophils, adhesion of embryonic cell lines, migration of eosinophils, quantity of endothelial cells, metabolism of phosphatidylcholine, binding of neutrophils, clearance of lipid, adhesion of epithelial cell lines, quantity of smooth muscle cells, adhesion of monocytes, recruitment of phospholipid, stimulation of endothelial tissue, surface area of atherosclerotic lesion, activation of inflammatory leukocytes, concentration of lipopolysaccharide, import of cholesterol, opsonization of cells, relaxation of coronary artery, synthesis of 12(S)-hydroxyeicosatetraenoic acid, lysis of blood clot, agglutination of blood cells, synthesis of 6-keto-prostaglandin F1 alpha, formation of plaque, release of phosphatidic acid, classical complement pathway, clearance of triacylglycerol, synthesis of corticosterone, activated partial thromboplastin time of plasma, auto-oxidation of amino acids, complement component C1s deficiency, delay in renal and urological disorder, density of artery, recruitment of phosphatidic acid. In some embodiments, the function is a physiological process.

As used herein, the term “function” is in reference to an organism, organ, a biological process, a biological system, a cellular process, a cellular system, a molecular process, a molecular system and the like, non-limiting examples of which include inflammatory response, lipid metabolism, redox signaling, immune response, endothelial dysfunction or any combination thereof. In some embodiments, the function is a physiological process.

In some embodiments, the function is inflammatory response and the proteins are any one or more of Alpha-2-macroglobulin (UniProt Accession No. P01023) (SEQ ID NO: 15), Angiotensinogen (UniProt Accession No. P01019) (SEQ ID NO: 14), Alpha-2-HS-glycoprotein (UniProt Accession No. P02765) (SEQ ID NO: 35), Apolipoprotein A-I (UniProt Accession No. P02647) (SEQ ID NO: 21), Apolipoprotein E (UniProt Accession No. P02649) (SEQ ID NO: 22), Complement C3 (UniProt Accession No. P01024) (SEQ ID NO: 16), Complement C5 (UniProt Accession No. P01031) (SEQ ID NO: 17), Complement factor H (UniProt Accession No. P08603) (SEQ ID NO: 56), C-reactive protein (UniProt Accession No. P02741) (SEQ ID NO: 28), Coagulation factor XII (UniProt Accession No. P00748) (SEQ ID NO: 9), Prothrombin (UniProt Accession No. P00734) (SEQ ID NO: 4), Vitamin D-binding protein (UniProt Accession No. P02774) (SEQ ID NO: 37), Gelsolin (UniProt Accession No. P06396) (SEQ ID NO: 51), Haptoglobin (UniProt Accession No. P00738) (SEQ ID NO: 6), Ig mu chain C region (UniProt Accession No. P01871) (SEQ ID NO: 19), Plasma kallikrein (UniProt Accession No. P03952) (SEQ ID NO: 40), Apolipoprotein(a) (UniProt Accession No. P08519) (SEQ ID NO: 55), Lumican (UniProt Accession No. P51884) (SEQ ID NO: 69), Alpha-1-acid glycoprotein 1 (UniProt Accession No. P02763) (SEQ ID NO: 34), Plasminogen (UniProt Accession No. P00747) (SEQ ID NO: 8), Serum paraoxonase/arylesterase 1 (UniProt Accession No. P27169) (SEQ ID NO: 65), Alpha-1-antitrypsin (UniProt Accession No. P01009) (SEQ ID NO: 12), Alpha-1-antichymotrypsin (UniProt Accession No. P01011) (SEQ ID NO: 13), Plasma protease C1 inhibitor (UniProt Accession No. P05155) (SEQ ID NO: 48), Vitronectin (UniProt Accession No. P04004) (SEQ ID NO: 42) or combinations thereof.

In some embodiments, the function is lipid metabolism and the proteins are any one or more of Angiotensinogen (UniProt Accession No. P01019) (SEQ ID NO: 14), Alpha-2-HS-glycoprotein (UnitProt Accession No. P02765) (SEQ ID NO: 35), Serum albumin (UniProt Accession No. P02768) (SEQ ID NO: 36), Apolipoprotein A-I (UniProt Accession No. P02647) (SEQ ID NO: 21), Apolipoprotein A-II (UniProt Accession No. P02652) (SEQ ID NO: 23), Apolipoprotein A-IV (UniProt Accession No. P06727) (SEQ ID NO: 52), Apolipoprotein B-100 (UniProt Accession No. P04114) (SEQ ID NO: 43), Apolipoprotein C-I (UniProt Accession No. P02654) (SEQ ID NO: 24), Apolipoprotein (UniProt Accession No. P02656) (SEQ ID NO: 26), Apolipoprotein E (UniProt Accession No. P02649) (SEQ ID NO: 22), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Complement factor H (UniProt Accession No. P08603) (SEQ ID NO: 56), Clusterin (UniProt Accession No. P10909) (SEQ ID NO: 61), C-reactive protein (UniProt Accession No. P02741) (SEQ ID NO: 28), Prothrombin (UniProt Accession No. P00734) (SEQ ID NO: 4), Vitamin D-binding protein (UniProt Accession No. P02774) (SEQ ID NO: 37), Haptoglobin (UniProt Accession No. P00738) (SEQ ID NO. 6), Plasma kallikrein (UniProt Accession No. P03952) (SEQ ID NO: 40), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), sex hormone binding globulin (UniProt Accession No. P04278) (SEQ ID NO: 46) or combinations thereof.

In some embodiments, the function is redox signaling and the proteins are any one or more of Angiotensinogen (UniProt Accession No. P01019) (SEQ ID NO: 14), Serum albumin (UniProt Accession No. P02768) (SEQ ID NO: 36), Apolipoprotein A-I (UniProt Accession No. P02647) (SEQ ID NO: 21), Apolipoprotein A-IV (UniProt Accession No. P06727) (SEQ ID NO: 52), Apolipoprotein (UniProt Accession No. P02655) (SEQ ID NO: 25), Apolipoprotein E (UniProt Accession No. P02649) (SEQ ID NO: 22), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Complement factor H (UniProt Accession No. P08603) (SEQ ID NO: 56), C-reactive protein (UniProt Accession No. P02741) (SEQ ID NO: 28), Prothrombin (UniProt Accession No. P00734) (SEQ ID NO: 4), Gelsolin (UniProt Accession No. P06396) (SEQ ID NO: 51), Hemoglobin subunit alpha (UniProt Accession No. P69905) (SEQ ID NO: 70), Haptoglobin (UniProt Accession No. P00738) (SEQ ID NO: 6), Inter-alpha-trypsin inhibitor heavy chain H4 (UniProt Accession No. Q14624) (SEQ ID NO: 71), Serum paraoxonase/lactonase 3 (UniProt Accession No. Q15166) (SEQ ID NO: 72), Peroxiredoxin-2 (UniProt Accession No. P32119) (SEQ ID NO: 66), Alpha-1-antitrypsin (UniProt Accession No. P01009) (SEQ ID NO: 12), Alpha-1-antichymotrypsin (UniProt Accession No. P01011) (SEQ ID NO: 13), Serotransferrin (UniProt Accession No. P02787) (SEQ ID NO: 38), or combinations thereof.

In some embodiments, the function is immune response and the proteins are any one or more of Complement C1q subcomponent subunit B (UniProt Accession No. P02746) (SEQ ID NO: 29), Complement C1q subcomponent subunit C (UniProt Accession No. P02747) (SEQ ID NO: 30), Complement C1r subcomponent (UniProt Accession No. P00736) (SEQ ID NO: 50), Complement C1s subcomponent (UniProt Accession No. P09871) (SEQ ID NO: 58), Complement C3 (UniProt Accession No. P01024) (SEQ ID NO: 16), Complement C4-A (UniProt Accession No. P0C0L4) (SEQ ID NO: 59), Complement C5 (UniProt Accession No. P01031) (SEQ ID NO: 17), Complement component C8 alpha chain (UniProt Accession No. P07357) (SEQ ID NO: 53), Complement component C9 (UniProt Accession No. P02748) (SEQ ID NO: 31), Complement factor B (UniProt Accession No. P00751) (SEQ ID NO: 10), Plasma protease C1 inhibitor (UniProt Accession No. P05155) (SEQ ID NO: 48), or combinations thereof.

In some embodiments, the function is endothelial dysfunction and the proteins are any one or more of Angiotensinogen (UniProt Accession No. P01019) (SEQ ID NO: 14), C-reactive protein (UniProt Accession No. P02741) (SEQ ID NO: 28), Lumican (UniProt Accession No. P51884) (SEQ ID NO: 69), Plasminogen (UniProt Accession No. P00747) (SEQ ID NO: 8), Peroxiredoxin-2 (UniProt Accession No. P32119) (SEQ ID NO: 66), Alpha-1-antitrypsin (UniProt Accession No. P01009) (SEQ ID NO: 12), Alpha-1-antichymotrypsin (UniProt Accession No. P01011) (SEQ ID NO: 13), Antithrombin-III (UniProt Accession No. P01008) (SEQ ID NO: 11), or combinations thereof.

In some embodiments, the function is coagulation and the proteins are any one or more of Serum albumin (UniProt Accession No. P02768) (SEQ ID NO: 36), Apolipoprotein E (UniProt Accession No. P02649) (SEQ ID NO: 22), Complement C3 (UniProt Accession No. P01024) (SEQ ID NO: 16), Complement C4-A (UniProt Accession No. P0C0L4) (SEQ ID NO: 59), Complement C4-B (UniProt Accession No. P0C0L5) (SEQ ID NO: 60), Complement C5 (UniProt Accession No. P01031) (SEQ ID NO: 17), Complement component C9 (UniProt Accession No. P02748) (SEQ ID NO: 31), Complement factor B (UniProt Accession No. P00751) (SEQ ID NO: 10), Complement factor H (UniProt Accession No.) (SEQ ID NO: 56), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Fibrinogen (UniProt Accession No. P02671) (SEQ ID NO: 27), Haptoglobin (UniProt Accession No. P00738) (SEQ ID NO: 6), Integrin alpha-Iib (UniProt Accession No. P08514) (SEQ ID NO: 54), Plasma kallikrein (UniProt Accession No. P03952) (SEQ ID NO: 40), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Peroxiredoxin-2 (UniProt Accession No. P32119) (SEQ ID NO: 66), Plasma serine protease inhibitor (UniProt Accession No. P05154) (SEQ ID NO: 47), Antithrombin-III (UniProt Accession No. P01008) (SEQ ID NO: 11), Heparin cofactor 2 (UniProt Accession No. P05546) (SEQ ID NO: 50), Alpha-2-antiplasmin (UniProt Accession No. P08697) (SEQ ID NO: 57), Vitronectin (UniProt Accession No. P04004) (SEQ ID NO: 42), von Willebrand factor (UniProt Accession No. P04275) (SEQ ID NO: 45), or combinations thereof.

In some embodiments, the function is lipid metabolism and the proteins are any one or more of Angiotensinogen (UniProt Accession No. P01019) (SEQ ID NO: 14), Alpha-2-HS-glycoprotein (UnitProt Accession No. P02765) (SEQ ID NO: 35), Serum albumin (UniProt Accession No. P02768) (SEQ ID NO: 36), Apolipoprotein A-I (UniProt Accession No. P02647) (SEQ ID NO: 21), Apolipoprotein A-II (UniProt Accession No. P02652) (SEQ ID NO: 23), Apolipoprotein A-IV (UniProt Accession No. P06727) (SEQ ID NO: 52), Apolipoprotein B-100 (UniProt Accession No. P04114) (SEQ ID NO: 43), Apolipoprotein C-I (UniProt Accession No. P02654) (SEQ ID NO: 24), Apolipoprotein (UniProt Accession No. P02656) (SEQ ID NO: 26), Apolipoprotein E (UniProt Accession No. P02649) (SEQ ID NO: 22), Zinc-alpha-2-glycoprotein (UniProt Accession No. P25311) (SEQ ID NO: 64), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Complement factor H (UniProt Accession No. P08603) (SEQ ID NO: 56), Clusterin (UniProt Accession No. P10909) (SEQ ID NO: 61), C-reactive protein (UniProt Accession No. P02741) (SEQ ID NO: 28), Prothrombin (UniProt Accession No. P00734) (SEQ ID NO: 4), Vitamin D-binding protein (UniProt Accession No. P02774) (SEQ ID NO: 37), Haptoglobin (UniProt Accession No. P00738) (SEQ ID NO. 6), Plasma kallikrein (UniProt Accession No. P03952) (SEQ ID NO: 40), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), Alpha-1-antitrypsin (UniProt Accession No. P01009) (SEQ ID NO: 12), sex hormone binding globulin (UniProt Accession No. P04278) (SEQ ID NO: 46) or combinations thereof.

In some embodiments, the function is inflammatory response and the proteins are any one or more of Alpha-2-macroglobulin (UniProt Accession No. P01023) (SEQ ID NO: 15), Angiotensinogen (UniProt Accession No. P01019) (SEQ ID NO: 14), Alpha-2-HS-glycoprotein (UniProt Accession No. P02765) (SEQ ID NO: 35), Apolipoprotein A-I (UniProt Accession No. P02647) (SEQ ID NO: 21), Apolipoprotein E (UniProt Accession No. P02649) (SEQ ID NO: 22), Complement C3 (UniProt Accession No. P01024) (SEQ ID NO: 16), Complement C5 (UniProt Accession No. P01031) (SEQ ID NO: 17), Complement factor H (UniProt Accession No. P08603) (SEQ ID NO: 56), C-reactive protein (UniProt Accession No. P02741) (SEQ ID NO: 28), C-reactive protein (UniProt Accession No. P02741) (SEQ ID NO: 28), Coagulation factor XII (UniProt Accession No. P00748) (SEQ ID NO: 9), Prothrombin (UniProt Accession No. P00734) (SEQ ID NO: 4), Vitamin D-binding protein (UniProt Accession No. P02774) (SEQ ID NO: 37), Gelsolin (UniProt Accession No. P06396) (SEQ ID NO: 51), Haptoglobin (UniProt Accession No. P00738) (SEQ ID NO: 6), Ig mu chain C region (UniProt Accession No. P01871) (SEQ ID NO: 19), Plasma kallikrein (UniProt Accession No. P03952) (SEQ ID NO: 40), Apolipoprotein(a) (UniProt Accession No. P08519) (SEQ ID NO: 55), Lumican (UniProt Accession No. P51884) (SEQ ID NO: 69), Alpha-1-acid glycoprotein 1 (UniProt Accession No. P02763) (SEQ ID NO: 34), Plasminogen (UniProt Accession No. P00747) (SEQ ID NO: 8), Serum paraoxonase/arylesterase 1 (UniProt Accession No. P27169) (SEQ ID NO: 65), Alpha-1-antitrypsin (UniProt Accession No. P01009) (SEQ ID NO: 12), Alpha-1-antichymotrypsin (UniProt Accession No. P01011) (SEQ ID NO: 13), Plasma protease C1 inhibitor (UniProt Accession No. P05155) (SEQ ID NO: 48), Vitronectin (UniProt Accession No. P04004) (SEQ ID NO: 42) or combinations thereof.

In some embodiments, the function is immune response and the proteins are any one or more of Angiotensinogen (UniProt Accession No. P01019) (SEQ ID NO: 14), Alpha-2-HS-glycoprotein (UniProt Accession No. P02765) (SEQ ID NO: 35), Apolipoprotein A-I (UniProt Accession No. P02647) (SEQ ID NO: 21), Apolipoprotein A-II (UniProt Accession No. P02652) (SEQ ID NO: 23), Apolipoprotein E (UniProt Accession No. P02649) (SEQ ID NO: 22), Complement C1q subcomponent subunit B (UniProt Accession No. P02746) (SEQ ID NO: 29), Complement C1q subcomponent subunit C (UniProt Accession No. P02747) (SEQ ID NO: 30), Complement C1r subcomponent (UniProt Accession No. P00736) (SEQ ID NO: 50), Complement C1s subcomponent (UniProt Accession No. P09871) (SEQ ID NO: 58), Complement C3 (UniProt Accession No. P01024) (SEQ ID NO: 16), Complement C4-A (UniProt Accession No. P0C0L4) (SEQ ID NO: 59), Complement C4-B (UniProt Accession No. P0C0L5) (SEQ ID NO: 60), Complement C5 (UniProt Accession No. P01031) (SEQ ID NO: 17), Complement component C8 alpha chain (UniProt Accession No. P07357) (SEQ ID NO: 53), Complement component C9 (UniProt Accession No. P02748) (SEQ ID NO: 31), Complement factor B (UniProt Accession No. P00751) (SEQ ID NO: 10), Complement factor H (UniProt Accession No. P08603) (SEQ ID NO: 56), C-reactive protein (UniProt Accession No. P02741) (SEQ ID NO: 28), Coagulation factor X (UniProt Accession No. P00742) (SEQ ID NO: 7), Gelsolin (UniProt Accession No. P06396) (SEQ ID NO: 51), Ig alpha-1 chain C region (UniProt Accession No. P01876) (SEQ ID NO: 20), Ig gamma-3 chain C region (UniProt Accession No. P01860) (SEQ ID NO: 18), Ig mu chain C region (UniProt Accession No. P01871) (SEQ ID NO: 19), Lumican (UniProt Accession No. P51884) (SEQ ID NO: 69), Serum paraoxonase/arylesterase 1 (UniProt Accession No. P27169) (SEQ ID NO: 65), Plasma protease C1 inhibitor (UniProt Accession No. P05155) (SEQ ID NO: 48), Vitronectin (UniProt Accession No. P04004) (SEQ ID NO:), von Willebrand factor (UniProt Accession No. P04275) (SEQ ID NO: 45), or combinations thereof

Diseases

Non-limiting examples of diseases include atherosclerosis, cardiac disease, lung damage, renal failure, endothelial dysfunction, cardiovascular disease, neurological disease, psychological disorders, developmental disorder, hereditary disorder, immunological disease, connective tissue disorders, skeletal disorders, muscular disorders. In some embodiments, the disease is a pathological process.

Non-limiting examples of diseases include disorders associated with function or development and/or disorders of a function or development, wherein the function or development is lipid metabolism, redox signaling, immune response, hematological system development, hematological system function, reproductive system development, reproductive system function, and gene expression. In some embodiments, the disease is a pathological process.

Other non-limiting examples of diseases include cancer, gastrointestinal disease, hepatic system disease, reproductive system disease, dermatological diseases, cell death, metabolic disease, neurological disease, immunological disease, hematological disease, psychological disorders, endocrine system disorders, connective tissue disorders, infectious diseases, hereditary disorder, respiratory disease, renal disease, urological disease, nutritional disease, and ophthalmic disease. In some embodiments, the disease is a pathological process.

Other non-limiting examples of diseases include disorders associated with function or development and/or disorders of a function or development, wherein the function or development is cellular movement, cellular growth, cell proliferation, tissue morphology, organismal survival, molecular transport, immune cell trafficking, cell-to-cell signaling, cell-to-cell interaction, lipid metabolism, small molecule biochemistry, tissue development, protein synthesis, free radical scavenging, protein degradation, protein synthesis, hematological system development, hematological system function, tissue morphology, carbohydrate metabolism, cellular function and maintenance, cell signaling, vitamin metabolism, mineral metabolism, digestive system development, digestive system function, hepatic system development, hepatic system function, hair development, hair function, skin development, skin function, nervous system development, nervous system function, cellular compromise, nucleic acid metabolism, small molecule biochemistry, organ morphology, organismal development, renal system development, renal system function, urological system development, urological system function, humoral immune response, amino acid metabolism, energy production, post-translational modification. In some embodiments, the disease is a pathological process.

Other non-limiting examples of diseases include Cardiovascular Disease, Hematological Disease, Hematological System Development and Function Disease, Neurological Disease, Organismal Injury and Abnormalities, Psychological Disorders, Developmental Disorder, Hereditary Disorder, Immunological Disease, Organismal Injury and Abnormalities, Cell-To-Cell Signaling and Interaction Disorder, Reproductive System Development and Function Disorder, Gene Expression Disorder, Cardiac Inflammation, Cardiovascular Disease, Connective Tissue Development and Function Disorder, Connective Tissue Disorders, Organismal Injury and Abnormalities, Hereditary Disorder, Skeletal and Muscular Disorders. In some embodiments, the disease is a pathological process.

Other non-limiting examples of diseases include abdominal cancer, urogenital cancer, abdominal adenocarcinoma, liver lesion, tumorigenesis of genital organ, genital tumor, pelvic cancer, female genital tract cancer, tumorigenesis of reproductive tract, liver cancer, skin lesion, melanoma, skin tumor, malignant cutaneous melanoma cancer, cell death, breast or ovarian cancer, lymphohematopoietic cancer, amyloidosis, glucose metabolism disorder, Lymphoid Cancer and Tumors, hematologic cancer, Dementia, lymphoproliferative malignancy, lymphoid cancer, Alzheimer's disease, diabetes mellitus, inflammation of organ, inflammation of absolute anatomical region, Rheumatic Disease, leukemia, inflammation of body cavity, ovarian cancer, Viral Infection, myeloproliferative disorder, occlusion of artery, chronic inflammatory disorder, atherosclerosis, systemic autoimmune syndrome, acute leukemia, bone marrow cancer, inflammation of joint, neuromuscular disease, acute myeloid leukemia, Thrombosis, Infarction, Movement Disorders, hemostasis, acute coronary syndrome, rheumatoid arthritis, disorder of basal ganglia, autosomal, recessive disease, coronary disease, peripheral vascular disease, Huntington's Disease, abnormal morphology of abdomen, blood clot, immunodeficiency, myocardial infarction, cerebrovascular dysfunction, Bacterial Infections, endocytosis, advanced malignant tumor, damage of lung, hemorrhagic disease, coronary artery disease, Fibrosis, Hypertension, autosomal dominant disease, acute myocardial infarction, injury of lung, systemic inflammatory response syndrome and/or sepsis, infiltration by neutrophils, renal impairment, vascular lesion, anemia, progressive motor neuropathy, disorder of pregnancy, sepsis, atherosclerotic lesion, failure of kidney, chemotaxis of myeloid cells, chemotaxis of phagocytes, inflammation of liver, accumulation of lipid, inflammation of intestine, blood protein disorder, obesity, adenoma, psoriasis, necrosis of epithelial tissue, complement component deficiency, damage of genitourinary system, chronic kidney disease, accumulation of blood cells, insulin resistance, Nephritis, colitis, non-insulin-dependent diabetes mellitus, thrombus, hyperlipoproteinemia, hemolysis, acute lung injury, damage of kidney, dyslipidemia, stroke, lupus erythematosus, cytopenia, Edema, chronic fatigue syndrome, atherogenesis, ischemia of brain, chronic renal failure, thrombocytopenia, diabetic complication, Polycystic Kidney Disease, damage of liver, retinal degeneration, age-related, macular degeneration, dengue fever, formation of blood clot, severe sepsis, Hypercholesterolemia, cholangiocarcinoma, hyperlipidemia, inflammation of pancreas, stenosis, septic shock, cirrhosis of liver, damage of central nervous system, cell death of endothelial cells, Wound, injury of liver, glomerulonephritis, fibrin clot, dengue hemorrhagic fever, thromboembolism, ischemia of heart, thrombosis of vein, infarction of cerebrum, hemolytic anemia, prostatic intraepithelial neoplasia, damage of bone, end stage renal disease, Parasitic Infection, apoptosis of endothelial cells, abnormality of left ventricle, cardiac lesion, venous thromboembolism, fibrinolysis, systemic inflammatory response syndrome, familial amyloidosis, atherosclerosis of aorta, formation of thrombus, bleeding of gastrointestinal tract, Hypertriglyceridemia, hereditary bleeding disorder, autoimmune, glomerulonephritis, cerebral ischemia, infection by Herpesviridae, sexually transmitted disease, metabolic syndrome X, hereditary angioedema, deep vein thrombosis, familial hypercholesterolemia, opsonization, microcytic anemia, unstable angina, bleeding of skin, ischemic stroke, chronic pancreatitis, malaria, albuminuria, angina pectoris, vasculitis, degradation of connective tissue, cerebral malaria, thrombophilia, atypical hemolytic uremic syndrome, familial amyloidotic polyneuropathy, systemic amyloidosis, hypolipoproteinemia, ST-elevation myocardial infarction, focal necrosis of liver, mixed hyperlipidemia, Stevens-Johnson syndrome, pulmonary embolism, agammaglobulinemia, infection by cytomegalovirus, disorder of menstruation, abnormal morphology of mesangial matrix, injury of intestine, activation of vascular endothelial cells, thrombosis of artery, hip fracture, immune response of peritoneal macrophages, acquired immunodeficiency syndrome, autoimmune pancreatitis, damage of blood vessel, purpura, benign thyroid nodule, familial dementia, abdominal aortic aneurysm, recurrent venous thrombosis, hemorrhagic stroke, heparin-induced thrombocytopenia, Finnish type amyloidosis, placental insufficiency, portal vein thrombosis, injury of myocardium, non-ST elevation myocardial infarction, acute immune thrombocytopenic purpura, chronic immune thrombocytopenic purpura, congenital agammaglobulinemia, fetal erythroblastosis, hereditary thrombophilia, recurrent sinopulmonary infection, chronic idiopathic thrombocytopenic purpura, thrombosis of kidney, vascular injury, abnormal aggregation of blood platelets, acute pulmonary embolism, neonatal alloimmune, thrombocytopenia, whooping cough, congenital heart block, familial combined hyperlipidemia, Devic's syndrome, toxic epidermal necrolysis, hematoma, hyperplasia of vascular smooth muscle cells, Rasmussen's encephalitis, hyper-igm immunodeficiency syndrome, chronic inflammatory demyelinating polyradiculoneuropathy, microalbuminuria, acute phase atypical hemolytic uremic syndrome, bleeding of abdominal aorta, recurrent deep vein thrombosis, recurrent thromboembolism, stage 2 acute myocardial infarction, transfusion related acute lung injury. In some embodiments, the disease is a pathological process.

Other non-limiting examples of diseases include disorders associated with function or development and/or disorders of a function or development, wherein the function or development is Acute Phase Response Signaling, LXR/RXR Activation, FXR/RXR Activation, Coagulation System, Complement System, Clathrin-mediated Endocytosis Signaling, Atherosclerosis Signaling, IL-12 Signaling and Production in Macrophages, Production of Nitric Oxide and Reactive Oxygen Species in Macrophages, Intrinsic Prothrombin Activation Pathway, Extrinsic Prothrombin Activation Pathway, Neuroprotective Role of THOP1 in Alzheimer's Disease, Hematopoiesis from Pluripotent Stem Cells, Primary Immunodeficiency Signaling, Systemic Lupus Erythematosus Signaling, Role of Pattern, Recognition Receptors in Recognition of Bacteria and Viruses, TR/RXR Activation, Role of Tissue Factor in Cancer, MSP-RON Signaling Pathway, Glioma Invasiveness Signaling, Caveolar-mediated Endocytosis Signaling, Communication between Innate and Adaptive Immune Cells and phagosome formation. In some embodiments, the disease is a pathological process.

Other non-limiting examples of diseases include disorders associated with function or development and/or disorders of a function or development, wherein the function or development is cell movement, proliferation of cells, migration of cells, quantity of cells, organismal death, development of vasculature, transport of molecule, leukocyte migration, inflammatory response, activation of cells, morphology of body cavity, adhesion of blood cells, concentration of lipid, generation of cells, movement of myeloid cells, cell movement of phagocytes, fatty acid metabolism, synthesis of lipid, angiogenesis, quantity of blood cells, cellular homeostasis, aggregation of cells, metabolism of protein, aggregation of blood cells, adhesion of immune cells, binding of cells, metabolism of reactive oxygen species, activation of blood cells, vasculogenesis, abnormal morphology of body cavity, synthesis of reactive oxygen species, cell movement of neutrophils, quantity of metal, catabolism of protein, neurological signs, activation of leukocytes, coagulation, aggregation of blood platelets, quantity of leukocytes, coagulation of blood, Bleeding, quantity of steroid, immune response of cells, survival of organism, transport of lipid, synthesis of fatty acid, size of lesion, engulfment of cells, cellular infiltration, chemotaxis, binding of blood cells, activation of myeloid cells, activation of phagocytes, quantity of carbohydrate, ion homeostasis of cells, growth of epithelial tissue, transport of steroid, export of molecule, concentration of sterol, cellular infiltration by leukocytes, quantity of Ca2+, quantity of phagocytes, quantity of myeloid cells, secretion of molecule, morphology of connective tissue, cytolysis, flux of Ca2+, immune response of leukocytes, accumulation of cells, recruitment of cells, endothelial cell development, adhesion of myeloid cells, generation of reactive oxygen species, migration of phagocytes, concentration of fatty acid, proliferation of endothelial cells, activation of antigen presenting cells, concentration of triacylglycerol, recruitment of leukocytes, production of reactive oxygen species, quantity of protein in blood, Organ Degeneration, efflux of cholesterol, adhesion of phagocytes, synthesis of eicosanoid, response of myeloid cells, phagocytosis of cells, concentration of cholesterol, metabolism of membrane lipid derivative, morphology of blood cells, binding of phagocytes, permeability of vasculature, synthesis of prostaglandin, homeostasis of lipid, release of lipid, immune response of phagocytes, activation of macrophages, steroid metabolism, recruitment of phagocytes, accumulation of leukocytes, proteolysis, cell movement of macrophages, recruitment of myeloid cells, endocytosis by eukaryotic cells, quantity of granulocytes, complement activation, transport of phospholipid, size of atherosclerotic lesion, binding of endothelial cells, binding of professional phagocytic cells, cell movement of monocytes, hydrolysis of lipid, quantity of reactive oxygen species, secretion of lipid, influx of Ca2+, Shock Response, quantity of neutrophils, synthesis of nitric oxide, mass of organism, function of cardiovascular system, efflux of phospholipid, esterification of cholesterol, adhesion of blood platelets, quantity of protein lipid complex in blood, experimentally, induced diabetes, permeability of blood vessel, adhesion of granulocytes, metabolism of hydrogen, peroxide, binding of myeloid cells, metabolism of acylglycerol, uptake of lipid, engulfment of leukocytes, accumulation of myeloid cells, immune response of antigen presenting cells, recruitment of granulocytes, internalization of cells, metabolism of phospholipid, molecular cleavage of protein fragment, synthesis of steroid, area of atherosclerotic lesion, abortion, binding of blood platelets, quantity of hdl cholesterol in blood, adhesion of neutrophils, chemotaxis of monocytes, biosynthesis of hydrogen peroxide, metabolism of triacylglycerol, activation of blood platelets, metabolism of cholesterol, cell movement of epithelial cell lines, activation of neuroglia, accumulation of phagocytes, migration of granulocytes, phagocytosis of phagocytes, phagocytosis of myeloid cells, conversion of lipid, migration of muscle cells, metabolism of phosphatidic acid, migration of antigen presenting cells, healing of wound, size of infarct, proliferation of liver cells, recruitment of neutrophils, quantity of macrophages, habitual abortion, secretion of triacylglycerol, quantity of superoxide, binding of vascular endothelial cells, peroxidation of lipid, activation of endothelial cells, quantity of blood vessel, injury of cells, binding of lipid, binding of carbohydrate, injury of kidney, homeostasis of cholesterol, response of granulocytes, cell movement of embryonic cell lines, activation of granulocytes, adhesion of epithelial cells, immune response of T lymphocytes, phagocytosis of antigen presenting cells, quantity of muscle, synthesis of prostaglandin E2, formation of blood vessel, release of eicosanoid, transport of heavy metal, cell movement of leukemia cell lines, cell movement of kidney cell lines, receptor-mediated endocytosis, migration of smooth muscle cells, transmission of lipid, concentration of cholesterol ester, respiratory burst of granulocytes, adhesion of kidney cell lines, neovascularization of choroid, quantity of ldl, cholesterol in blood, relaxation of artery, recruitment of monocytes, function of blood platelets, synthesis of cyclic GMP, flow of blood, binding of polysaccharide, synthesis of cholesterol, binding of, antigen presenting cells, generation of superoxide, production of hydrogen peroxide, accumulation of macrophages, immune response of neutrophils, formation of reactive oxygen species, release of arachidonic acid, growth of bacteria, activation of neutrophils, quantity of heavy metal, damage of connective tissue, release of cholesterol, uptake of cholesterol ester, activation of carbohydrate, coagulation of plasma, quantity of lysophosphatidylcholine, metabolism of lipoprotein, internalization of lipid, movement of macrophage cancer cell lines, binding of cell surface, hydrolysis of triacylglycerol, respiratory burst of neutrophils, adhesion of embryonic cell lines, migration of eosinophils, quantity of endothelial cells, metabolism of phosphatidylcholine, binding of neutrophils, clearance of lipid, adhesion of epithelial cell lines, quantity of smooth muscle cells, adhesion of monocytes, recruitment of phospholipid, stimulation of endothelial tissue, surface area of atherosclerotic lesion, activation of inflammatory leukocytes, concentration of lipopolysaccharide, import of cholesterol, opsonization of cells, relaxation of coronary artery, synthesis of 12(S)-hydroxyeicosatetraenoic acid, lysis of blood clot, agglutination of blood cells, synthesis of 6-keto-prostaglandin F1 alpha, formation of plaque, release of phosphatidic acid, classical complement pathway, clearance of triacylglycerol, synthesis of corticosterone, activated partial thromboplastin time of plasma, auto-oxidation of amino acids, complement component C1s deficiency, delay in renal and urological disorder, density of artery, recruitment of phosphatidic acid. In some embodiments, the disease is a pathological process.

In some embodiments, the disease is atherosclerosis, renal failure, cardiovascular disease or any combination thereof.

In some embodiments, the disease is atherosclerosis and the proteins are any one or more of Angiotensinogen (UniProt Accession No. P01019) (SEQ ID NO: 14), Alpha-2-HS-glycoprotein (UniProt Accession No. P02765) (SEQ ID NO: 35), Apolipoprotein A-I (UniProt Accession No. P02647) (SEQ ID NO: 21), Apolipoprotein A-II (Uniprot Accession No. P02652) (SEQ ID NO: 23), Apolipoprotein A-IV (UniProt Accession No. P06727) (SEQ ID NO: 52), Apolipoprotein B-100 (UniProt Accession No. P04114) (SEQ ID NO: 43), Apolipoprotein C-I (UniProt Accession No. P02654) (SEQ ID NO: 24), Apolipoprotein C-II (UniProt Accession No. P02655) (SEQ ID NO: 25), Apolipoprotein (UniProt Accession No. P02656) (SEQ ID NO. 26), Apolipoprotein E (UniProt Accession No. P02649) (SEQ ID NO: 22), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Prothrombin (UniProt Accession No. P00734) (SEQ ID NO: 4), Hemoglobin subunit alpha (UniProt Accession No. P69905) (SEQ ID NO: 70), Haptoglobin (UniProt Accession No. P00738) (SEQ ID NO: 6), Integrin alpha-Iib (UniProt Accession No. P08514) (SEQ ID NO: 54), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), Peroxiredoxin-2 (UniProt Accession No. P32119) (SEQ ID NO: 66), Antithrombin-III (UniProt Accession No. P01008) (SEQ ID NO. 11), Heparin cofactor 2 (UniProt Accession No. P05546) (SEQ ID NO: 50), von Willebrand factor (UniProt Accession No. P04275) (SEQ ID NO: 45) or combinations thereof.

In some embodiments, the disease is renal failure and the proteins are any one or more of Angiotensinogen (UniProt Accession No. P01019) (SEQ ID NO: 14), Serum albumin (UniProt Accession No. P02768) (SEQ ID NO: 36), Apolipoprotein B-100 (UniProt Accession No. P04114) (SEQ ID NO: 43), Complement C3 (UniProt Accession No. P01024) (SEQ ID NO: 16), Complement C4-A (UniProt Accession No. P0C0L4) (SEQ ID NO: 59), Complement C5 (UniProt Accession No. P01031) (SEQ ID NO: 17), Complement factor B (UniProt Accession No. P00751) (SEQ ID NO: 10), C-reactive protein (UniProt Accession No. P02741) (SEQ ID NO: 28), Coagulation factor X (UniProt Accession No. P00742) (SEQ ID NO: 7), Prothrombin (UniProt Accession No. P00734) (SEQ ID NO: 4), Hemoglobin subunit alpha (UniProt Accession No. P69905) (SEQ ID NO: 70), Antithrombin-III (UniProt Accession No. P01008) (SEQ ID NO: 11), Serotransferrin (UniProt Accession No. P02787) (SEQ ID NO: 38), or combinations thereof.

In some embodiments, the cardiovascular disease is selected from congestive heart failure, arrhythmia, pericarditis, acute myocardial infarction, infarcted myocardium, coronary artery disease, coronary heart disease, ischemic heart disease, cardiomyopathy, stroke, hypertensive heart disease, heart failure, pulmonary heart disease, ischemic syndrome, coronary microvascular disease, cardiac dysrhythmias, rheumatic heart disease, aortic aneurysms, cardiomyopathy, atrial fibrillation, congenital heart disease, endocarditis, inflammatory heart disease, inflammatory cardiomegaly, myocarditis, valvular heart disease, cerebrovascular disease, peripheral artery disease or any combination thereof.

In some embodiments, the disease is cardiovascular disease and the proteins are any one or more of Angiotensinogen (UniProt Accession No. P01019) (SEQ ID NO: 14), Apolipoprotein A-I (UniProt Accession No. P02647) (SEQ ID NO: 21), Apolipoprotein B-100 (UniProt Accession No. P04114) (SEQ ID NO: 43), Apolipoprotein E (UniProt Accession No. P02649) (SEQ ID NO: 22), Complement C3 (UniProt Accession No. P01024) (SEQ ID NO: 16), Complement C4-A (UniProt Accession No. P0C0L4) (SEQ ID NO: 59), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Gelsolin (UniProt Accession No. P06396) (SEQ ID NO: 51), Integrin alpha-Iib (UniProt Accession No. P08514) (SEQ ID NO: 54), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Antithrombin-III (UniProt Accession No. P01008) (SEQ ID NO: 11), Plasma protease C1 inhibitor (UniProt Accession No. P05155) (SEQ ID NO: 48), von Willebrand factor (UniProt Accession No. P04275) (SEQ ID NO: 45), or combinations thereof.

In some embodiments, the disease is cardiovascular disease and the proteins are any one or more of Angiotensinogen (UniProt Accession No. P01019) (SEQ ID NO: 14), Apolipoprotein A-I (UniProt Accession No. P02647) ((SEQ ID NO: 21), Apolipoprotein B-100 (UniProt Accession No. P04114) (SEQ ID NO: 43), Apolipoprotein E (UniProt Accession No. P02649) (SEQ ID NO: 22), Complement C3 (UniProt Accession No. P01024) (SEQ ID NO: 16), Complement C4-A (UniProt Accession No. P0C0L4) (SEQ ID NO: 59), Complement C4-B (UniProt Accession No. P0C0L5) (SEQ ID NO: 60), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Gelsolin (UniProt Accession No. P06396) (SEQ ID NO: 51), Integrin alpha-Iib (UniProt Accession No. P08514) (SEQ ID NO: 54), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Antithrombin-III Accession No. P01008) (SEQ ID NO: 11), Alpha-2-antiplasmin (UniProt Accession No. P08697) (SEQ ID NO: 57), Plasma protease C1 inhibitor (UniProt Accession No. P05155) (SEQ ID NO: 48), von Willebrand factor (UniProt Accession No. P04275) (SEQ ID NO: 45), or combinations thereof.

In some embodiments, the disease is liver disease and the proteins are any one or more of Alpha-1B-glycoprotein (UniProt Accession No. P04217) (SEQ ID NO: 44), Alpha-2-macroglobulin (UniProt Accession No. P01023) (SEQ ID NO: 15), Afamin (UniProt Accession No. P43652) (SEQ ID NO: 68), Angiotensinogen (UniProt Accession No. P01019) (SEQ ID NO: 14), Alpha-2-HS-glycoprotein (UniProt Accession No. P02765) (SEQ ID NO: 35), Serum albumin (UniProt Accession No. P02768) (SEQ ID NO: 36), Protein AMBP (UniProt Accession No. P02760) (SEQ ID NO: 33), Apolipoprotein A-I (UniProt Accession No. P02647) (SEQ ID NO: 21), Apolipoprotein A-II (UniProt Accession No. P02652) (SEQ ID NO: 23), Apolipoprotein B-100 (UniProt Accession No. P04114) (SEQ ID NO: 43), Apolipoprotein C-I (UniProt Accession No. P02654) (SEQ ID NO: 24), Apolipoprotein E (UniProt Accession No. P02649) (SEQ ID NO: 22), Apolipoprotein L1 (UniProt Accession No. 014791) (SEQ ID NO: 1), Complement C1q subcomponent subunit C (UniProt Accession No. P02747) (SEQ ID NO: 30), Complement C1r subcomponent (UniProt Accession No. P00736) (SEQ ID NO: 5), Complement C1s subcomponent (UniProt Accession No. P09871) (SEQ ID NO: 58), Complement C3 (UniProt Accession No. P01024) (SEQ ID NO: 16), Complement C4-A (UniProt Accession No. P0C0L4) (SEQ ID NO: 59), Complement C4-B (UniProt Accession No. P0C0L5) (SEQ ID NO: 60), C4b-binding protein alpha chain (UniProt Accession No. P04003) (SEQ ID NO: 41), Complement C5 (UniProt Accession No. P01031) (SEQ ID NO: 17), Complement component C8 alpha chain (UniProt Accession No. P07357) (SEQ ID NO: 53), Complement component C9 (UniProt Accession No. P02748) (SEQ ID NO: 31), Complement factor B (UniProt Accession No. P00751) (SEQ ID NO: 10), Complement factor H (UniProt Accession No. P08603) (SEQ ID NO: 56), Clusterin (UniProt Accession No. P10909) (SEQ ID NO: 61), Ceruloplasmin (UniProt Accession No. P00450) (SEQ ID NO: 2), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Fibrinogen (UniProt Accession No. P02671) (SEQ ID NO: 27), Vitamin D-binding protein (UniProt Accession No. P02774) (SEQ ID NO: 37), Gelsolin (UniProt Accession No. P06396) (SEQ ID NO: 51), Hemoglobin subunit alpha (UniProt Accession No. P69905) (SEQ ID NO: 70), Haptoglobin (UniProt Accession No. P00738) (SEQ ID NO: 6), Hemopexin (UniProt Accession No. P02790) (SEQ ID NO: 39), Integrin alpha-Iib (UniProt Accession No. P08514) (SEQ ID NO: 54), Inter-alpha-trypsin inhibitor heavy chain H1 (UniProt Accession No. P19827) (SEQ ID NO: 63), Inter-alpha-trypsin inhibitor heavy chain H2 (UniProt Accession No. P19823) (SEQ ID NO: 62), Inter-alpha-trypsin inhibitor heavy chain H4 (UniProt Accession No. Q14624) (SEQ ID NO: 71), Plasma kallikrein (UniProt Accession No. P03952) (SEQ ID NO: 40), Apolipoprotein(a) (UniProt Accession No. P08519) (SEQ ID NO: 55), Leucine-rich alpha-2-glycoprotein (UniProt Accession No. P02750) (SEQ ID NO: 32), Lumican (UniProt Accession No. P51884) (SEQ ID NO: 69), Alpha-1-acid glycoprotein 1 (UniProt Accession No. P02763) (SEQ ID NO: 34), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), Peroxiredoxin-2 (UniProt Accession No. P32119) (SEQ ID NO: 66), Serum amyloid A-4 protein (UniProt Accession No. P35542) (SEQ ID NO: 67), Alpha-1-antitrypsin (UniProt Accession No. P01009) (SEQ ID NO: 12), Alpha-1-antichymotrypsin (UniProt Accession No. P01011) (SEQ ID NO: 13), Antithrombin-III (UniProt Accession No. P01008) (SEQ ID NO: 11), Heparin cofactor 2 (UniProt Accession No. P05546) (SEQ ID NO: 50), Plasma protease C1 inhibitor (UniProt Accession No. P05155) (SEQ ID NO: 48), Serotransferrin (UniProt Accession No. P02787) (SEQ ID NO: 38), Vitronectin (UniProt Accession No. P04004) (SEQ ID NO: 42), von Willebrand factor (UniProt Accession No. P04275) (SEQ ID NO: 45), or combinations thereof.

In some embodiments, the disease is vascular disease and the proteins are any one or more of Angiotensinogen (UniProt Accession No. P01019) (SEQ ID NO: 14), Alpha-2-HS-glycoprotein (UniProt Accession No. P02765) (SEQ ID NO: 35), Apolipoprotein A-I UniProt Accession No. P02647) (SEQ ID NO: 21), Apolipoprotein A-II (UniProt Accession No.) (SEQ ID NO: 23), Apolipoprotein A-IV (UniProt Accession No. P06727) (SEQ ID NO: 52), Apolipoprotein B-100 (UniProt Accession No. P04114) (SEQ ID NO: 43), Apolipoprotein C-I (UniProt Accession No. P02654) (SEQ ID NO: 24), Apolipoprotein C-II (UniProt Accession No. P02655) (SEQ ID NO: 25), Apolipoprotein (UniProt Accession No. P02656) (SEQ ID NO: 26), Apolipoprotein E (UniProt Accession No. P02649) (SEQ ID NO: 22), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Prothrombin (UniProt Accession No. P00734) (SEQ ID NO: 4), Hemoglobin subunit alpha (UniProt Accession No. P69905) (SEQ ID NO: 70), Haptoglobin (UniProt Accession No. P00738) (SEQ ID NO: 6), Integrin alpha-Iib (UniProt Accession No. P08514) (SEQ ID NO: 54), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Serum paraoxonase/lactonase 3 (UniProt Accession No. Q15166) (SEQ ID NO: 72), Peroxiredoxin-2 (UniProt Accession No. P32119) (SEQ ID NO: 66), Antithrombin-III (UniProt Accession No. P01008) (SEQ ID NO: 11), Heparin cofactor 2 (UniProt Accession No. P05546) (SEQ ID NO: 50), von Willebrand factor (UniProt Accession No. P04275) (SEQ ID NO: 45), or combinations thereof.

In some embodiments, the disease is lung damage and the proteins are any one or more of Alpha-2-HS-glycoprotein (UniProt Accession No. P02765) (SEQ ID NO: 35), Serum albumin (UniProt Accession No. P02768) (SEQ ID NO: 36), Protein AMBP (UniProt Accession No. P02760) (SEQ ID NO: 33), Zinc-alpha-2-glycoprotein (UniProt Accession No. P25311) (SEQ ID NO: 64), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Clusterin (UniProt Accession No. P10909) (SEQ ID NO: 61), C-reactive protein (UniProt Accession No. P02741) (SEQ ID NO: 28), Coagulation factor XII (UniProt Accession No. P00748 (SEQ ID NO: 9), Vitamin D-binding protein (UniProt Accession No. P02774) (SEQ ID NO: 37), Hemoglobin subunit alpha (UniProt Accession No. P69905) (SEQ ID NO: 70), Plasma protease C1 inhibitor (UniProt Accession No. P05155) (SEQ ID NO; 48), Serotransferrin (UniProt Accession No. P02787) (SEQ ID NO: 38), von Willebrand factor (UniProt Accession No. P04275) (SEQ ID NO: 45), or combinations thereof.

In some embodiments, the disease is lung damage and the proteins are any one or more of Alpha-2-HS-glycoprotein (UniProt Accession No. P02765) (SEQ ID NO: 35), Serum albumin (UniProt Accession No. P02768) (SEQ ID NO: 36), Protein AMBP (UniProt Accession No. P02760) (SEQ ID NO: 33), Zinc-alpha-2-glycoprotein (UniProt Accession No. P25311) (SEQ ID NO: 64), Complement C3 (UniProt Accession No. P01024) (SEQ ID NO: 16), Complement C5 (UniProt Accession No. P01031) (SEQ ID NO: 17), Complement factor B (UniProt Accession No. P00751) (SEQ ID NO: 10), Clusterin (UniProt Accession No. P10909) (SEQ ID NO: 61), Vitamin D-binding protein (UniProt Accession No. P02774) (SEQ ID NO: 37), Hemoglobin subunit alpha (UniProt Accession No. P69905) (SEQ ID NO: 70), Plasma protease C1 inhibitor (UniProt Accession No. P05155) (SEQ ID NO; 48), Serotransferrin (UniProt Accession No. P02787) (SEQ ID NO: 38), von Willebrand factor (UniProt Accession No. P04275) (SEQ ID NO: 45), or combinations thereof

Protein Panels

In various embodiments, the present invention provides a protein panel, the panel comprising, consisting essentially of, or consisting of one or more proteins listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

In various embodiments, the protein panel comprises, consists essentially of, or consists of 1 or more; 2 or more; 3 or more; 4 or more; 5 or more; 6 or more; 7 or more; 8 or more; 9 or more; 10 or more; 11 or more; 12 or more; 13 or more; 14 or more; 15 or more; 16 or more; 17 or more; 18 or more; 19 or more; 20 or more; 21 or more; 22 or more; 23 or more; 24 or more; 25 or more; 26 or more; 27 or more; 28 or more; 29 or more; 30 or more; 31 or more; 32 or more; 33 or more; 34 or more; 35 or more; 36 or more; 37 or more; 38 or more; 39 or more; 40 or more; 41 or more; 42 or more; 43 or more; 44 or more; 45 or more; 46 or more; 47 or more; 48 or more; 49 or more; 50 or more; 51 or more; 52 or more; 53 or more; 54 or more; 55 or more; 56 or more; 57 or more; 58 or more; 59 or more; 60 or more; 61 or more; 62 or more; 63 or more; 64 or more; 65 or more; 66 or more; 67 or more; 68 or more; 69 or more; 70 or more; 71 or more; or 72; or all of the proteins listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

In various embodiments, the present invention provides a protein panel, the panel comprising, consisting essentially of, or consisting of the proteins listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

In various embodiments, the present invention provides a protein panel for assessing and/or determining the state of health of a subject, the panel comprising one or more proteins listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

In some embodiments, the one or more proteins are biomarkers for one or more functions or combination thereof. In some embodiments, the functions are cardiovascular function, immune mediated inflammation, lipid metabolism, redox signaling, immune response, hematological system development, hematological system function, reproductive system development, reproductive system function, gene expression, etc. In some embodiments, the function is selected from cardiovascular function, immune mediated inflammation, lipid metabolism, redox signaling, immune response, hematological system development, hematological system function, reproductive system development, reproductive system function, and gene expression.

In some embodiments, the one or more proteins are biomarkers for one or more functions or combination thereof. In some embodiments, the functions are inflammatory response, lipid metabolism, redox signaling, lung damage, immune response, or endothelial dysfunction.

In some embodiments, the one or more proteins are biomarkers for one or more diseases or combination thereof. In some embodiments, the disease is atherosclerosis, renal failure, cardiovascular disease or any combination thereof.

In some embodiments, the protein panel is a biomarker panel. In some embodiments, the protein panel is a protein biomarker panel.

In some embodiments, the protein panel can be used for any one or more of the following: identifying one or more functions in a subject; diagnosing one or more diseases in a subject; assessing and/or determining the state of health of a subject; determining the prognosis of a subject; determining the efficacy of a treatment of a subject; identifying the presence, absence, amount, or level of one or more proteins in a sample; obtaining a biomarker signature for a sample; and/or determining the risk of developing one or more diseases in a subject.

In various embodiments, the present invention provides a protein panel for assessing and/or determining the state of health of a subject; diagnosing a disease in a subject; assessing and/or determining the risk of developing a disease in a subject; prognosing a disease of a subject; identifying and/or detecting one or more proteins in a sample; the panel comprising one or more proteins listed in Table 15.

Methods of Obtaining a Biomarker Signature

In various embodiments, the present invention provides a method for obtaining a biomarker signature for a subject, comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; and correlating the one or more peptides to one or more proteins so as to obtain the biomarker signature for the subject, wherein the one or more proteins are listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

In some embodiments, the biomarker signature comprises, consists essentially of, or consists of none; 1 or more; 2 or more; 3 or more; 4 or more; 5 or more; 6 or more; 7 or more; 8 or more; 9 or more; 10 or more; 11 or more; 12 or more; 13 or more; 14 or more; 15 or more; 16 or more; 17 or more; 18 or more; 19 or more; 20 or more; 21 or more; 22 or more; 23 or more; 24 or more; 25 or more; 26 or more; 27 or more; 28 or more; 29 or more; 30 or more; 31 or more; 32 or more; 33 or more; 34 or more; 35 or more; 36 or more; 37 or more; 38 or more; 39 or more; 40 or more; 41 or more; 42 or more; 43 or more; 44 or more; 45 or more; 46 or more; 47 or more; 48 or more; 49 or more; 50 or more; 51 or more; 52 or more; 53 or more; 54 or more; 55 or more; 56 or more; 57 or more; 58 or more; 59 or more; 60 or more; 61 or more; 62 or more; 63 or more; 64 or more; 65 or more; 66 or more; 67 or more; 68 or more; 69 or more; 70 or more; 71 or more; or 72; or all of the proteins listed in Table 15. In some embodiments, the biomarker signature is the biomarker signature for the subject. In some embodiments, the biomarker signature is the protein biomarker signature of the subject. In some embodiments, the biomarker signature is the reference biomarker signature. In some embodiments, the biomarker signature is the reference protein biomarker signature.

In some embodiments, the biomarker signature comprises, consists essentially of, or consists of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 of the proteins listed in Table 15.

In some embodiments, the biomarker signature from the subject comprises, consists essentially of, or consists of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 of the proteins listed in Table 15.

In some embodiments, the biomarker signature from the reference sample comprises, consists essentially of, or consists of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 of the proteins listed in Table 15.

In some embodiments, the reference biomarker signature comprises, consists essentially of, or consists of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 of the proteins listed in Table 15.

In some embodiments, the sample from the subject comprises, consists essentially of, or consists of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 of the proteins listed in Table 15.

In some embodiments, the reference sample comprises, consists essentially of, or consists of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 of the proteins listed in Table 15.

In some embodiments, the sample from the subject comprises, consists essentially of, or consists of none; 1 or more; 2 or more; 3 or more; 4 or more; 5 or more; 6 or more; 7 or more; 8 or more; 9 or more; 10 or more; 11 or more; 12 or more; 13 or more; 14 or more; 15 or more; 16 or more; 17 or more; 18 or more; 19 or more; 20 or more; 21 or more; 22 or more; 23 or more; 24 or more; 25 or more; 26 or more; 27 or more; 28 or more; 29 or more; 30 or more; 31 or more; 32 or more; 33 or more; 34 or more; 35 or more; 36 or more; 37 or more; 38 or more; 39 or more; 40 or more; 41 or more; 42 or more; 43 or more; 44 or more; 45 or more; 46 or more; 47 or more; 48 or more; 49 or more; 50 or more; 51 or more; 52 or more; 53 or more; 54 or more; 55 or more; 56 or more; 57 or more; 58 or more; 59 or more; 60 or more; 61 or more; 62 or more; 63 or more; 64 or more; 65 or more; 66 or more; 67 or more; 68 or more; 69 or more; 70 or more; 71 or more; or 72; or all of the proteins listed in Table 15.

In some embodiments, the reference sample, comprises, consists essentially of, or consists of none; 1 or more; 2 or more; 3 or more; 4 or more; 5 or more; 6 or more; 7 or more; 8 or more; 9 or more; 10 or more; 11 or more; 12 or more; 13 or more; 14 or more; 15 or more; 16 or more; 17 or more; 18 or more; 19 or more; 20 or more; 21 or more; 22 or more; 23 or more; 24 or more; 25 or more; 26 or more; 27 or more; 28 or more; 29 or more; 30 or more; 31 or more; 32 or more; 33 or more; 34 or more; 35 or more; 36 or more; 37 or more; 38 or more; 39 or more; 40 or more; 41 or more; 42 or more; 43 or more; 44 or more; 45 or more; 46 or more; 47 or more; 48 or more; 49 or more; 50 or more; 51 or more; 52 or more; 53 or more; 54 or more; 55 or more; 56 or more; 57 or more; 58 or more; 59 or more; 60 or more; 61 or more; 62 or more; 63 or more; 64 or more; 65 or more; 66 or more; 67 or more; 68 or more; 69 or more; 70 or more; 71 or more; or 72; or all of the proteins listed in Table 15.

In some embodiments, the biomarker signature is a protein biomarker signature.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 5. In some embodiments, the mass spectrometry data comprises one or more Q1/Q3 mass value pairs, wherein the Q1/Q3 mass value pairs are correlated to the one or more peptides, and the one or more peptides are correlated to the one or more proteins according to Table 5.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 7. In some embodiments, the mass spectrometry data comprises one or more precursor ions and one or more product ions, wherein the precursor ions and the product ions are correlated to the one or more peptides, and the one or more peptides are correlated to the one or more proteins according to Table 7.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 8. In some embodiments, the mass spectrometry data comprises one or more precursor ions and one or more product ions, wherein the precursor ions and the product ions are correlated to the one or more peptides, and the one or more peptides are correlated to the one or more proteins according to Table 8.

In some embodiments, the mass spectrometry is parallel reaction monitoring (PRM) mass spectrometry. In some embodiments, the one or more peptides are correlated to the one or more proteins according to Table 9. In some embodiments, the mass spectrometry data comprises one or more precursor ions and one or more product ions, wherein the precursor ions and the product ions are correlated to the one or more peptides, and the one or more peptides are correlated to the one or more proteins according to Table 9.

In some embodiments, the mass spectrometry is parallel reaction monitoring (PRM) mass spectrometry. In some embodiments, the one or more peptides are correlated to the one or more proteins according to Table 10. In some embodiments, the mass spectrometry data comprises one or more precursor ions and one or more product ions, wherein the precursor ions and the product ions are correlated to the one or more peptides, and the one or more peptides are correlated to the one or more proteins according to Table 10.

In some embodiments, the mass spectrometry is data dependent acquisition (DDA) mass spectrometry. In some embodiments, the one or more peptides are correlated to the one or more proteins according to Table 11. In some embodiments, the mass spectrometry data comprises one or more precursor ions, wherein the precursor ions are correlated to the one or more peptides, and the one or more peptides are correlated to the one or more proteins according to Table 11.

In some embodiments, the mass spectrometry is data dependent acquisition (DDA) mass spectrometry. In some embodiments, the one or more peptides are correlated to the one or more proteins according to Table 12. In some embodiments, the mass spectrometry data comprises one or more precursor ions and one or more product ions, wherein the precursor ions and the product ions are correlated to the one or more peptides, and the one or more peptides are correlated to the one or more proteins according to Table 12.

In some embodiments, the mass spectrometry is data independent acquisition (DIA) mass spectrometry. In some embodiments, the one or more peptides are correlated to the one or more proteins according to Table 13. In some embodiments, the mass spectrometry data comprises one or more precursor ions and one or more product ions, wherein the precursor ions and the product ions are correlated to the one or more peptides, and the one or more peptides are correlated to the one or more proteins according to Table 13.

In some embodiments, the mass spectrometry is data independent acquisition (DIA) mass spectrometry. In some embodiments, the one or more peptides are correlated to the one or more proteins according to Table 14. In some embodiments, the mass spectrometry data comprises one or more precursor ions and one or more product ions, wherein the precursor ions and the product ions are correlated to the one or more peptides, and the one or more peptides are correlated to the one or more proteins according to Table 14.

In some embodiments, one or more proteases are any one or more of trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof. In some embodiments, the protease is trypsin.

In some embodiments, the method further comprises adding one or more internal standards to the sample. In some embodiments, the internal standard comprises one or more stable isotope-labeled peptide standard, one or more isotopically labeled peptides, one or more isotopically labeled proteins, or any combination thereof.

Methods for Determining State of Health

In various embodiments, the present invention provides a method for assessing and/or determining the state of health of a subject, the method comprising: obtaining a sample from a subject, contacting the sample with one or more proteases so as to obtain a digested sample, wherein the digested sample comprises one or more peptides, analyzing the digested sample by mass spectrometry so as to obtain mass spectrometry data; correlating the mass spectrometry data to the one or more peptides; and correlating the one or more peptides to one or more proteins, wherein the one or more proteins are listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

and; comparing the presence or amount or level of the one or more proteins in the sample from the subject to the presence or amount or level of the one or more proteins in a reference sample so as to assess and/or determine the state of health of the subject. In some embodiments, the method further comprises measuring and/or detecting the presence or an amount or level of one or more proteins in the sample from the subject.

In some embodiments, the presence of one or more proteins in the sample from the subject relative to the reference sample is indicative of a poor state of health. In some embodiments, an increase in the amount or level of one or more proteins in the sample from the subject relative to the reference sample is indicative of a poor state of health. In some embodiments, a decrease in the amount or level of one or more proteins in the sample from the subject relative to the reference sample is indicative of a poor state of health. In some embodiments, a change in the amount or level of one or more proteins in the sample from the subject relative to the reference sample is indicative of a poor state of health. In some embodiments, the absence of one or more proteins in the sample from the subject relative to the reference sample is indicative of a good state of health. In some embodiments, the absence of one or more proteins in the sample from the subject relative to the reference sample is indicative of wellness.

In various embodiments, the present invention provides a method for assessing and/or determining the state of health of a subject; comprising: obtaining a sample from the subject; measuring and/or detecting one or more proteins in the sample by mass spectrometry so as to obtain a biomarker signature for the subject, wherein the one or more proteins are listed in Table 15; comparing the biomarker signature from the subject to one or more reference biomarker signatures; and diagnosing the subject based on the comparison, In some embodiments, the method further comprises treating the subject and/or administering a treatment and/or selecting a treatment and/or prescribing a treatment and/or providing a treatment and/or administering a preventative treatment and/or selecting a preventative treatment and/or prescribing a preventative treatment and/or providing a preventative treatment. In some embodiments, the one or more reference biomarker signatures are from one or more subjects having one or more diseases. In some embodiments, the one or more reference biomarker signatures are from one or more healthy subjects. In some embodiments, the subject is diagnosed with the disease if the comparison of the biomarker signature from the subject to one or more reference biomarker signatures shows a change or difference in the biomarker signature from the subject relative to one or more reference biomarker signatures. In some embodiments, the subject is diagnosed with the disease if the comparison of the biomarker signature from the subject to one or more reference biomarker signatures does not show a change or difference in the biomarker signature from the subject relative to one or more reference biomarker signatures. In some embodiments, the detecting one or more proteins in the sample by mass spectrometry comprises analyzing the digested sample by mass spectrometry so as to obtain mass spectrometry data; correlating the mass spectrometry data to the one or more peptides; and correlating the one or more peptides to one or more proteins, wherein the one or more proteins are listed in Table 15. In some embodiments, the digested sample is obtained by contacting the sample with one or more proteases so as to obtain a digested sample, wherein the digested sample comprises one or more peptides.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 5. In some embodiments, the mass spectrometry data comprises one or more Q1/Q3 mass value pairs, wherein the Q1/Q3 mass value pairs are correlated to the one or more peptides, and the one or more peptides are correlated to the one or more proteins according to Table 5.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 7. In some embodiments, the mass spectrometry data comprises one or more precursor ions and one or more product ions, wherein the precursor ions and the product ions are correlated to the one or more peptides, and the one or more peptides are correlated to the one or more proteins according to Table 7.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 8. In some embodiments, the mass spectrometry data comprises one or more precursor ions and one or more product ions, wherein the precursor ions and the product ions are correlated to the one or more peptides, and the one or more peptides are correlated to the one or more proteins according to Table 8.

In some embodiments, the mass spectrometry is parallel reaction monitoring (PRM) mass spectrometry. In some embodiments, the one or more peptides are correlated to the one or more proteins according to Table 9. In some embodiments, the mass spectrometry data comprises one or more precursor ions and one or more product ions, wherein the precursor ions and the product ions are correlated to the one or more peptides, and the one or more peptides are correlated to the one or more proteins according to Table 9.

In some embodiments, the mass spectrometry is parallel reaction monitoring (PRM) mass spectrometry. In some embodiments, the one or more peptides are correlated to the one or more proteins according to Table 10. In some embodiments, the mass spectrometry data comprises one or more precursor ions and one or more product ions, wherein the precursor ions and the product ions are correlated to the one or more peptides, and the one or more peptides are correlated to the one or more proteins according to Table 10.

In some embodiments, the mass spectrometry is data dependent acquisition (DDA) mass spectrometry. In some embodiments, the one or more peptides are correlated to the one or more proteins according to Table 11. In some embodiments, the mass spectrometry data comprises one or more precursor ions, wherein the precursor ions are correlated to the one or more peptides, and the one or more peptides are correlated to the one or more proteins according to Table 11.

In some embodiments, the mass spectrometry is data dependent acquisition (DDA) mass spectrometry. In some embodiments, the one or more peptides are correlated to the one or more proteins according to Table 12. In some embodiments, the mass spectrometry data comprises one or more precursor ions and one or more product ions, wherein the precursor ions and the product ions are correlated to the one or more peptides, and the one or more peptides are correlated to the one or more proteins according to Table 12.

In some embodiments, the mass spectrometry is data independent acquisition (DIA) mass spectrometry. In some embodiments, the one or more peptides are correlated to the one or more proteins according to Table 13. In some embodiments, the mass spectrometry data comprises one or more precursor ions and one or more product ions, wherein the precursor ions and the product ions are correlated to the one or more peptides, and the one or more peptides are correlated to the one or more proteins according to Table 13.

In some embodiments, the mass spectrometry is data independent acquisition (DIA) mass spectrometry. In some embodiments, the one or more peptides are correlated to the one or more proteins according to Table 14. In some embodiments, the mass spectrometry data comprises one or more precursor ions and one or more product ions, wherein the precursor ions and the product ions are correlated to the one or more peptides, and the one or more peptides are correlated to the one or more proteins according to Table 14.

In some embodiments, one or more proteases are any one or more of trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof. In some embodiments, the protease is trypsin.

In some embodiments, the method further comprises adding one or more internal standards to the sample. In some embodiments, the internal standard comprises one or more stable isotope-labeled peptide standard, one or more isotopically labeled peptides, one or more isotopically labeled proteins, or any combination thereof.

Prognostic Methods

In various embodiments, the present invention provides a method for determining the prognosis of a subject, the method comprising: obtaining a sample from a subject, contacting the sample with one or more proteases so as to obtain a digested sample, wherein the digested sample comprises one or more peptides, analyzing the digested sample by mass spectrometry so as to obtain mass spectrometry data, correlating the mass spectrometry data to the one or more peptides; and correlating the one or more peptides to one or more proteins, wherein the one or more proteins are listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

and; comparing the presence or amount or level of the one or more proteins in the sample from the subject to the presence or amount or level of the one or more proteins in a reference sample so as to determine the prognosis of the subject. In some embodiments, the method further comprises measuring and/or detecting the presence or an amount or level of one or more proteins in the sample from the subject.

In some embodiments, the presence of one or more proteins in the sample from the subject relative to the reference sample is indicative of a poor prognosis. In some embodiments, an increase in the amount or level of one or more proteins in the sample from the subject relative to the reference sample is indicative of a poor prognosis. In some embodiments, a decrease in the amount or level of one or more proteins in the sample from the subject relative to the reference sample is indicative of a poor prognosis. In some embodiments, a change in the amount or level of one or more proteins in the sample from the subject relative to the reference sample is indicative of a poor prognosis. In some embodiments, the absence of one or more proteins in the sample from the subject relative to the reference sample is indicative of a good prognosis.

In some embodiments, the method further comprises treating the subject and/or selecting a treatment for and/or providing a treatment to the subject based on the prognosis.

In various embodiments, the present invention provides a method for determining the prognosis of a subject, comprising: obtaining a sample from a subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; and correlating the one or more peptides to one or more proteins so as to obtain a protein biomarker signature for the subject, wherein the one or more proteins are listed in Table 15; and comparing the protein biomarker signature from the subject to one or more reference protein biomarker signatures so as to obtain the prognosis of the subject. In some embodiments, a change in the protein biomarker signature from the subject relative to one or more reference biomarker signatures is indicative of a poor prognosis. In some embodiments, the reference protein biomarker signature is obtained from a control subject, wherein the control subject does not have the disease. In some embodiments, the reference protein biomarker signature is obtained from the subject before the subject is treated for the disease. In some embodiments, the reference protein biomarker signature is from a subject that has been successfully treated for the disease. In some embodiments, the method further comprises treating the subject and/or selecting a treatment for and/or providing a treatment to the subject based on the prognosis.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 5. In some embodiments, the mass spectrometry data comprises one or more Q1/Q3 mass value pairs, wherein the Q1/Q3 mass value pairs are correlated to the one or more peptides, and the one or more peptides are correlated to the one or more proteins according to Table 5.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 7. In some embodiments, the mass spectrometry data comprises one or more precursor ions and one or more product ions, wherein the precursor ions and the product ions are correlated to the one or more peptides, and the one or more peptides are correlated to the one or more proteins according to Table 7.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 8. In some embodiments, the mass spectrometry data comprises one or more precursor ions and one or more product ions, wherein the precursor ions and the product ions are correlated to the one or more peptides, and the one or more peptides are correlated to the one or more proteins according to Table 8.

In some embodiments, the mass spectrometry is parallel reaction monitoring (PRM) mass spectrometry. In some embodiments, the one or more peptides are correlated to the one or more proteins according to Table 9. In some embodiments, the mass spectrometry data comprises one or more precursor ions and one or more product ions, wherein the precursor ions and the product ions are correlated to the one or more peptides, and the one or more peptides are correlated to the one or more proteins according to Table 9.

In some embodiments, the mass spectrometry is parallel reaction monitoring (PRM) mass spectrometry. In some embodiments, the one or more peptides are correlated to the one or more proteins according to Table 10. In some embodiments, the mass spectrometry data comprises one or more precursor ions and one or more product ions, wherein the precursor ions and the product ions are correlated to the one or more peptides, and the one or more peptides are correlated to the one or more proteins according to Table 10.

In some embodiments, the mass spectrometry is data dependent acquisition (DDA) mass spectrometry. In some embodiments, the one or more peptides are correlated to the one or more proteins according to Table 11. In some embodiments, the mass spectrometry data comprises one or more precursor ions, wherein the precursor ions are correlated to the one or more peptides, and the one or more peptides are correlated to the one or more proteins according to Table 11.

In some embodiments, the mass spectrometry is data dependent acquisition (DDA) mass spectrometry. In some embodiments, the one or more peptides are correlated to the one or more proteins according to Table 12. In some embodiments, the mass spectrometry data comprises one or more precursor ions and one or more product ions, wherein the precursor ions and the product ions are correlated to the one or more peptides, and the one or more peptides are correlated to the one or more proteins according to Table 12.

In some embodiments, the mass spectrometry is data independent acquisition (DIA) mass spectrometry. In some embodiments, the one or more peptides are correlated to the one or more proteins according to Table 13. In some embodiments, the mass spectrometry data comprises one or more precursor ions and one or more product ions, wherein the precursor ions and the product ions are correlated to the one or more peptides, and the one or more peptides are correlated to the one or more proteins according to Table 13.

In some embodiments, the mass spectrometry is data independent acquisition (DIA) mass spectrometry. In some embodiments, the one or more peptides are correlated to the one or more proteins according to Table 14. In some embodiments, the mass spectrometry data comprises one or more precursor ions and one or more product ions, wherein the precursor ions and the product ions are correlated to the one or more peptides, and the one or more peptides are correlated to the one or more proteins according to Table 14.

In some embodiments, one or more proteases are any one or more of trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof. In some embodiments, the protease is trypsin.

In some embodiments, the method further comprises adding one or more internal standards to the sample. In some embodiments, the internal standard comprises one or more stable isotope-labeled peptide standard, one or more isotopically labeled peptides, one or more isotopically labeled proteins, or any combination thereof.

Diagnostic Methods

In various embodiments, the present invention provides a method for diagnosing a disease in a subject, comprising: obtaining a sample from a subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; and correlating the one or more peptides to one or more proteins so as to obtain a protein biomarker signature for the subject, wherein the one or more proteins are listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

and; comparing the protein biomarker signature from the subject to one or more reference protein biomarker signatures, wherein a change in the protein biomarker signature from the subject relative to one or more reference biomarker signatures is indicative of the disease in the subject.

In some embodiments, the reference protein biomarker signature is obtained from a control subject, wherein the control subject does not have the disease. In some embodiments, the reference protein biomarker signature is obtained from the subject before the subject is treated for the disease. In some embodiments, the reference protein biomarker signature is from a subject that has been successfully treated for the disease.

In some embodiments, the method further comprises treating the subject and/or selecting a treatment for and/or providing a treatment to the subject based on the diagnosis.

In various embodiments, the present invention provides a method for assessing the efficacy of the treatment, comprising: comparing the protein biomarker signature from the subject to one or more reference protein biomarker signatures, wherein a change in the protein biomarker signature from the subject relative to one or more reference protein biomarker signatures is indicative of the efficacy of the treatment.

In some embodiments, the method further comprises determining that the subject does not have the disease; and selecting and/or providing a preventative treatment for the subject.

In some embodiments, the method further comprises determining that the subject has the disease; and treating the subject and/or selecting a treatment for and/or providing a treatment for the subject and/or prescribing a treatment for the subject and/or administering a treatment to the subject.

In various embodiments, the present invention provides a method for diagnosing a disease in a subject; comprising: obtaining a sample from the subject; measuring and/or detecting one or more proteins in the sample by mass spectrometry so as to obtain a biomarker signature for the subject, wherein the one or more proteins are listed in Table 15; comparing the biomarker signature from the subject to one or more reference biomarker signatures; and diagnosing the subject based on the comparison, In some embodiments, the method further comprises treating the subject and/or administering a treatment and/or selecting a treatment and/or prescribing a treatment and/or providing a treatment and/or administering a preventative treatment and/or selecting a preventative treatment and/or prescribing a preventative treatment and/or providing a preventative treatment. In some embodiments, the one or more reference biomarker signatures are from one or more subjects having one or more diseases. In some embodiments, the one or more reference biomarker signatures are from one or more healthy subjects. In some embodiments, the subject is diagnosed with the disease if the comparison of the biomarker signature from the subject to one or more reference biomarker signatures shows a change or difference in the biomarker signature from the subject relative to one or more reference biomarker signatures. In some embodiments, the subject is diagnosed with the disease if the comparison of the biomarker signature from the subject to one or more reference biomarker signatures does not show a change or difference in the biomarker signature from the subject relative to one or more reference biomarker signatures.

In various embodiments, the present invention provides a method for diagnosing a disease in a subject, comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins, wherein the one or more proteins are listed in Table 15; comparing an amount or level of the one or more proteins in the sample from the subject to an amount or level of the one or more proteins in a reference sample; wherein an increase in the amount or level of the one or more proteins in the sample from the subject relative to the amount or level of the one or more proteins from the reference sample is indicative of and/or a diagnosis of the disease in the subject. In some embodiments, the method further comprises measuring and/or detecting an amount or level of one or more proteins in the sample from the subject. In some embodiments, the method further comprises selecting or prescribing a treatment for the subject based on the diagnosis. In some embodiments, the method further comprises treating the subject based on the diagnosis. In some embodiments, the method further comprises administering a treatment to the subject based on the diagnosis. In some embodiments, the method further comprises providing a treatment to the subject based on the diagnosis. In some embodiments, the method further comprises referring the subject to a specialist based on the diagnosis. In some embodiments, the sample is obtained before, during, or after treatment for the disease. In some embodiments the reference sample is obtained from a control subject, wherein the control subject does not have the disease. In some embodiments, the reference sample is obtained from a subject that has been successfully treated for the disease. In some embodiments, the reference sample is a sample obtained from the subject at an earlier point in time. In some embodiments, the reference sample is obtained from the subject before the subject is treated for the disease. In some embodiments, the proteins in the reference sample are one or more proteins in Table 15.

In various embodiments, the present invention provides a method for diagnosing a disease in a subject; comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins, wherein the one or more proteins are listed in Table 15; comparing an amount or level of the one or more proteins in the sample from the subject to an amount or level of the one or more proteins in a reference sample; wherein a decrease in the amount or level of the one or more proteins in the sample from the subject relative to the amount or level of the one or more proteins from the reference sample is indicative of and/or a diagnosis of the disease in the subject. In some embodiments, the method further comprises measuring and/or detecting an amount or level of one or more proteins in the sample from the subject. In some embodiments, the method further comprises selecting or prescribing a treatment for the subject based on the diagnosis. In some embodiments, the method further comprises treating the subject based on the diagnosis. In some embodiments, the method further comprises administering a treatment to the subject based on the diagnosis. In some embodiments, the method further comprises providing a treatment to the subject based on the diagnosis. In some embodiments, the method further comprises referring the subject to a specialist based on the diagnosis. In some embodiments, the sample is obtained before, during, or after treatment for the disease. In some embodiments the reference sample is obtained from a control subject, wherein the control subject does not have the disease. In some embodiments, the reference sample is obtained from a subject that has been successfully treated for the disease. In some embodiments, the reference sample is a sample obtained from the subject at an earlier point in time. In some embodiments, the reference sample is obtained from the subject before the subject is treated for the disease. In some embodiments, the proteins in the reference sample are one or more proteins in Table 15.

In various embodiments, the present invention provides a method for diagnosing a disease in a subject; comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins, wherein the one or more proteins are listed in Table 15; and comparing an amount or level of the one or more proteins in the sample from the subject to an amount or level of the one or more proteins in a reference sample, wherein a change in the amount or level of the one or more proteins in the sample from the subject relative to the amount or level of the one or more proteins from the reference sample is indicative of and/or a diagnosis of the disease in the subject. In some embodiments, the method further comprises measuring and/or detecting an amount or level of one or more proteins in the sample from the subject. In some embodiments, the method further comprises selecting or prescribing a treatment for the subject based on the diagnosis. In some embodiments, the method further comprises treating the subject based on the diagnosis. In some embodiments, the method further comprises administering a treatment to the subject based on the diagnosis. In some embodiments, the method further comprises providing a treatment to the subject based on the diagnosis. In some embodiments, the method further comprises referring the subject to a specialist based on the diagnosis. In some embodiments, the sample is obtained before, during, or after treatment for the disease. In some embodiments the reference sample is obtained from a control subject, wherein the control subject does not have the disease. In some embodiments, the reference sample is obtained from a subject that has been successfully treated for the disease. In some embodiments, the reference sample is a sample obtained from the subject at an earlier point in time. In some embodiments, the reference sample is obtained from the subject before the subject is treated for the disease. In some embodiments, the proteins in the reference sample are one or more proteins in Table 15.

In various embodiments, the present invention provides a method for diagnosing a disease in a subject; comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins, wherein the one or more proteins are listed in Table 15; and detecting a presence of the one or more proteins in the sample from the subject, wherein the presence of the one or more proteins in the sample from the subject relative to a reference sample is indicative of and/or a diagnosis of the disease in the subject. In some embodiments, the method further comprises selecting or prescribing a treatment for the subject based on the diagnosis. In some embodiments, the method further comprises treating the subject based on the diagnosis. In some embodiments, the method further comprises administering a treatment to the subject based on the diagnosis. In some embodiments, the method further comprises providing a treatment to the subject based on the diagnosis. In some embodiments, the method further comprises referring the subject to a specialist based on the diagnosis. In some embodiments, the sample is obtained before, during, or after treatment for the disease. In some embodiments the reference sample is obtained from a control subject, wherein the control subject does not have the disease. In some embodiments, the reference sample is obtained from a subject that has been successfully treated for the disease. In some embodiments, the reference sample is a sample obtained from the subject at an earlier point in time. In some embodiments, the reference sample is obtained from the subject before the subject is treated for the disease.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 5. In some embodiments, the mass spectrometry data comprises one or more Q1/Q3 mass value pairs, wherein the Q1/Q3 mass value pairs are correlated to the one or more peptides, and the one or more peptides are correlated to the one or more proteins according to Table 5.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 7. In some embodiments, the mass spectrometry data comprises one or more precursor ions and one or more product ions, wherein the precursor ions and the product ions are correlated to the one or more peptides, and the one or more peptides are correlated to the one or more proteins according to Table 7.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 8. In some embodiments, the mass spectrometry data comprises one or more precursor ions and one or more product ions, wherein the precursor ions and the product ions are correlated to the one or more peptides, and the one or more peptides are correlated to the one or more proteins according to Table 8.

In some embodiments, the mass spectrometry is parallel reaction monitoring (PRM) mass spectrometry. In some embodiments, the one or more peptides are correlated to the one or more proteins according to Table 9. In some embodiments, the mass spectrometry data comprises one or more precursor ions and one or more product ions, wherein the precursor ions and the product ions are correlated to the one or more peptides, and the one or more peptides are correlated to the one or more proteins according to Table 9.

In some embodiments, the mass spectrometry is parallel reaction monitoring (PRM) mass spectrometry. In some embodiments, the one or more peptides are correlated to the one or more proteins according to Table 10. In some embodiments, the mass spectrometry data comprises one or more precursor ions and one or more product ions, wherein the precursor ions and the product ions are correlated to the one or more peptides, and the one or more peptides are correlated to the one or more proteins according to Table 10.

In some embodiments, the mass spectrometry is data dependent acquisition (DDA) mass spectrometry. In some embodiments, the one or more peptides are correlated to the one or more proteins according to Table 11. In some embodiments, the mass spectrometry data comprises one or more precursor ions, wherein the precursor ions are correlated to the one or more peptides, and the one or more peptides are correlated to the one or more proteins according to Table 11.

In some embodiments, the mass spectrometry is data dependent acquisition (DDA) mass spectrometry. In some embodiments, the one or more peptides are correlated to the one or more proteins according to Table 12. In some embodiments, the mass spectrometry data comprises one or more precursor ions and one or more product ions, wherein the precursor ions and the product ions are correlated to the one or more peptides, and the one or more peptides are correlated to the one or more proteins according to Table 12.

In some embodiments, the mass spectrometry is data independent acquisition (DIA) mass spectrometry. In some embodiments, the one or more peptides are correlated to the one or more proteins according to Table 13. In some embodiments, the mass spectrometry data comprises one or more precursor ions and one or more product ions, wherein the precursor ions and the product ions are correlated to the one or more peptides, and the one or more peptides are correlated to the one or more proteins according to Table 13.

In some embodiments, the mass spectrometry is data independent acquisition (DIA) mass spectrometry. In some embodiments, the one or more peptides are correlated to the one or more proteins according to Table 14. In some embodiments, the mass spectrometry data comprises one or more precursor ions and one or more product ions, wherein the precursor ions and the product ions are correlated to the one or more peptides, and the one or more peptides are correlated to the one or more proteins according to Table 14.

In some embodiments, one or more proteases are any one or more of trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof. In some embodiments, the protease is trypsin.

In some embodiments, the method further comprises adding one or more internal standards to the sample. In some embodiments, the internal standard comprises one or more stable isotope-labeled peptide standard, one or more isotopically labeled peptides, one or more isotopically labeled proteins, or any combination thereof.

Treatment Methods

In various embodiments, the present invention provides a method for treating, inhibiting, and/or reducing the severity of a disease in a subject, comprising: selecting a subject diagnosed with one or more diseases by the methods described herein and administering to the subject a treatment so as to treat, inhibit, and/or reduce the severity of the disease.

In various embodiments, the present invention provides a method of treating, inhibiting, and/or reducing the severity of a disease in a subject, comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins so as to obtain a protein biomarker signature for the subject, wherein the one or more proteins are listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

comparing the protein biomarker signature from the subject to one or more reference protein biomarker signatures, wherein a change in the protein biomarker signature from the subject relative to the reference protein biomarker signatures is a diagnosis of one or more diseases in the subject; and selecting, prescribing, and/or administering a treatment and/or therapy to the subject based on the diagnosis. In some embodiments, the change in the protein biomarker signature is an increase in the level of one or more proteins in the sample from the subject (or the protein biomarker signature from the subject) relative to a reference sample (or the reference protein biomarker signatures). In some embodiments, the change in the protein biomarker signature is a decrease in the level of one or more proteins in the sample from the subject (or the protein biomarker signature from the subject) relative to a reference sample (or the reference protein biomarker signatures).

In various embodiments, the present invention provides a method of treating, inhibiting, and/or reducing the severity of a disease in a subject, comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins, wherein the one or more proteins are listed in Table 15; measuring the presence of the one or more proteins in the sample from the subject; comparing the presence of the one or more proteins in the sample from the subject to the presence of the one or more proteins in a reference sample, wherein the presence of one or more proteins in the sample from the subject relative to the reference sample is a diagnosis of one or more diseases in the subject; and selecting, prescribing, and/or administering a treatment and/or therapy to the subject based on the diagnosis.

In various embodiments, the present invention provides a method of treating, inhibiting, and/or reducing the severity of a disease in a subject, comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins, wherein the one or more proteins are listed in Table 15; measuring an amount or level of the one or more proteins in the sample from the subject; and comparing the amount or level of the one or more proteins in the sample from the subject to an amount or level of the one or more proteins in a reference sample, wherein an increase in the level of one or more proteins in the sample from the subject relative to the reference sample is a diagnosis of one or more diseases in the subject; and selecting, prescribing, and/or administering a treatment and/or therapy to the subject based on the diagnosis.

In various embodiments, the present invention provides a method of treating, inhibiting, and/or reducing the severity of a disease in a subject, comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins, wherein the one or more proteins are listed in Table 15; measuring an amount or level of the one or more proteins in the sample from the subject; comparing the amount or level of the one or more proteins in the sample from the subject to an amount or level of the one or more proteins in a reference sample, wherein a decrease in the level of one or more proteins in the sample from the subject relative to the reference sample is a diagnosis of one or more diseases in the subject; and selecting, prescribing, and/or administering a treatment and/or therapy to the subject based on the diagnosis.

In various embodiments, the present invention provides a method of treating, inhibiting, and/or reducing the severity of a disease in a subject, comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins, wherein the one or more proteins are listed in Table 15; measuring an amount or level of the one or more proteins in the sample from the subject; and comparing the amount or level of the one or more proteins in the sample from the subject to an amount or level of the one or more proteins in a reference sample, wherein a change in the level of one or more proteins in the sample from the subject relative to the reference sample is a diagnosis of one or more diseases in the subject; and selecting, prescribing, and/or administering a treatment and/or therapy to the subject based on the diagnosis.

In some embodiments, the method further comprises assessing the efficacy of the treatment, comprising: comparing the amount or level of the one or more proteins in the sample from the subject to an amount or level of the one or more proteins in a reference sample, wherein a decrease in the amount or level of the one or more proteins in the sample from the subject relative to the amount or level from the reference sample is indicative of the efficacy of the treatment.

In some embodiments, the method further comprises assessing the efficacy of the treatment, comprising: comparing the amount or level of the one or more proteins in the sample from the subject to an amount or level of the one or more proteins in a reference sample, wherein an increase in the amount or level of the one or more proteins in the sample from the subject relative to the amount or level from the reference sample is indicative of the efficacy of the treatment.

In some embodiments, the method further comprises assessing the efficacy of the treatment, comprising: comparing the amount or level of the one or more proteins in the sample from the subject to an amount or level of the one or more proteins in a reference sample, wherein a change in the amount or level of the one or more proteins in the sample from the subject relative to the amount or level from the reference sample is indicative of the efficacy of the treatment.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 5. In some embodiments, the mass spectrometry data comprises one or more Q1/Q3 mass value pairs, wherein the Q1/Q3 mass value pairs are correlated to the one or more peptides, and the one or more peptides are correlated to the one or more proteins according to Table 5.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 7. In some embodiments, the mass spectrometry data comprises one or more precursor ions and one or more product ions, wherein the precursor ions and the product ions are correlated to the one or more peptides, and the one or more peptides are correlated to the one or more proteins according to Table 7.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 8. In some embodiments, the mass spectrometry data comprises one or more precursor ions and one or more product ions, wherein the precursor ions and the product ions are correlated to the one or more peptides, and the one or more peptides are correlated to the one or more proteins according to Table 8.

In some embodiments, the mass spectrometry is parallel reaction monitoring (PRM) mass spectrometry. In some embodiments, the one or more peptides are correlated to the one or more proteins according to Table 9. In some embodiments, the mass spectrometry data comprises one or more precursor ions and one or more product ions, wherein the precursor ions and the product ions are correlated to the one or more peptides, and the one or more peptides are correlated to the one or more proteins according to Table 9.

In some embodiments, the mass spectrometry is parallel reaction monitoring (PRM) mass spectrometry. In some embodiments, the one or more peptides are correlated to the one or more proteins according to Table 10. In some embodiments, the mass spectrometry data comprises one or more precursor ions and one or more product ions, wherein the precursor ions and the product ions are correlated to the one or more peptides, and the one or more peptides are correlated to the one or more proteins according to Table 10.

In some embodiments, the mass spectrometry is data dependent acquisition (DDA) mass spectrometry. In some embodiments, the one or more peptides are correlated to the one or more proteins according to Table 11. In some embodiments, the mass spectrometry data comprises one or more precursor ions, wherein the precursor ions are correlated to the one or more peptides, and the one or more peptides are correlated to the one or more proteins according to Table 11.

In some embodiments, the mass spectrometry is data dependent acquisition (DDA) mass spectrometry. In some embodiments, the one or more peptides are correlated to the one or more proteins according to Table 12. In some embodiments, the mass spectrometry data comprises one or more precursor ions and one or more product ions, wherein the precursor ions and the product ions are correlated to the one or more peptides, and the one or more peptides are correlated to the one or more proteins according to Table 12.

In some embodiments, the mass spectrometry is data independent acquisition (DIA) mass spectrometry. In some embodiments, the one or more peptides are correlated to the one or more proteins according to Table 13. In some embodiments, the mass spectrometry data comprises one or more precursor ions and one or more product ions, wherein the precursor ions and the product ions are correlated to the one or more peptides, and the one or more peptides are correlated to the one or more proteins according to Table 13.

In some embodiments, the mass spectrometry is data independent acquisition (DIA) mass spectrometry. In some embodiments, the one or more peptides are correlated to the one or more proteins according to Table 14. In some embodiments, the mass spectrometry data comprises one or more precursor ions and one or more product ions, wherein the precursor ions and the product ions are correlated to the one or more peptides, and the one or more peptides are correlated to the one or more proteins according to Table 14.

In some embodiments, one or more proteases are any one or more of trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof. In some embodiments, the protease is trypsin.

In some embodiments, the method further comprises adding one or more internal standards to the sample. In some embodiments, the internal standard comprises one or more stable isotope-labeled peptide standard, one or more isotopically labeled peptides, one or more isotopically labeled proteins, or any combination thereof.

Methods for Assessing and/or Determining Risk of Developing a Disease

In various embodiments, the present invention provides a method for assessing and/or determining the risk of developing a disease in a subject; comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins so as to obtain a protein biomarker signature for the subject, wherein the one or more proteins are listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

and; comparing the protein biomarker signature from the subject to one or more reference protein biomarker signatures, wherein a change in the protein biomarker signature from the subject relative to the reference protein biomarker signatures is indicative of an increased risk of the subject developing the disease. In some embodiments, the method further comprises selecting or prescribing a treatment for the subject based on the determination. In some embodiments, the method further comprises treating the subject based on the determination. In some embodiments, the method further comprises administering a treatment to the subject based on the determination. In some embodiments, the method further comprises providing a treatment to the subject based on the determination. In some embodiments, the method further comprises referring the subject to a specialist based on the determination. In some embodiments, the sample is obtained before, during, or after treatment for the disease. In some embodiments the reference biomarker signature is obtained from a control subject, wherein the control subject does not have the disease. In some embodiments, the reference biomarker signature is obtained from a subject that has been successfully treated for the disease. In some embodiments, the reference biomarker signature is a biomarker signature obtained from the subject at an earlier point in time. In some embodiments, the reference protein biomarker signature is obtained from the subject before the subject is treated for the disease.

In various embodiments, the present invention provides a method for assessing and/or determining the risk of developing a disease in a subject; comprising: obtaining a sample from the subject; measuring one or more proteins in the sample by mass spectrometry so as to obtain a biomarker signature for the subject, wherein the one or more proteins are listed in Table 15; comparing the biomarker signature from the subject to one or more reference biomarker signatures; and assessing and/or determining the risk of the subject developing the disease based on the comparison, In some embodiments, the method further comprises treating the subject and/or administering a treatment and/or selecting a treatment and/or prescribing a treatment and/or providing a treatment and/or administering a preventative treatment and/or selecting a preventative treatment and/or prescribing a preventative treatment and/or providing a preventative treatment. In some embodiments, the one or more reference biomarker signatures are from one or more subjects having one or more diseases. In some embodiments, the one or more reference biomarker signatures are from one or more healthy subjects. In some embodiments, the subject is determined to be at risk of developing the disease if the comparison of the biomarker signature from the subject to one or more reference biomarker signatures shows a change or difference in the biomarker signature form the subject relative to one or more reference biomarker signatures.

In various embodiments, the present invention provides a method for assessing and/or determining the risk of developing a disease in a subject; comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins, wherein the one or more proteins are listed in Table 15; comparing an amount or level of the one or more proteins in the sample from the subject to an amount or level of the one or more proteins in a reference sample; and determining that the subject has increased risk of developing the disease if the amount or level of the one or more proteins in the sample is increased relative to the reference sample. In some embodiments, the method further comprises measuring an amount or level of one or more proteins in the sample from the subject. In some embodiments, the method further comprises selecting or prescribing a treatment for the subject based on the determination. In some embodiments, the method further comprises treating the subject based on the determination. In some embodiments, the method further comprises administering a treatment to the subject based on the determination. In some embodiments, the method further comprises providing a treatment to the subject based on the determination. In some embodiments, the method further comprises referring the subject to a specialist based on the determination. In some embodiments, the sample is obtained before, during, or after treatment for the disease. In some embodiments the reference sample is obtained from a control subject, wherein the control subject does not have the disease. In some embodiments, the reference sample is obtained from a subject that has been successfully treated for the disease. In some embodiments, the reference sample is a sample obtained from the subject at an earlier point in time. In some embodiments, the reference sample is obtained from the subject before the subject is treated for the disease.

In various embodiments, the present invention provides a method for assessing and/or determining the risk of developing a disease in a subject; comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins, wherein the one or more proteins are listed in Table 15; comparing an amount or level of the one or more proteins in the sample from the subject to an amount or level of the one or more proteins in a reference sample; and determining that the subject has increased risk of developing the disease if the amount or level of the one or more proteins in the sample is decreased relative to the reference sample. In some embodiments, the method further comprises measuring an amount or level of one or more proteins in the sample from the subject. In some embodiments, the method further comprises selecting or prescribing a treatment for the subject based on the determination. In some embodiments, the method further comprises treating the subject based on the determination. In some embodiments, the method further comprises administering a treatment to the subject based on the determination. In some embodiments, the method further comprises providing a treatment to the subject based on the determination. In some embodiments, the method further comprises referring the subject to a specialist based on the determination. In some embodiments, the sample is obtained before, during, or after treatment for the disease. In some embodiments the reference sample is obtained from a control subject, wherein the control subject does not have the disease. In some embodiments, the reference sample is obtained from a subject that has been successfully treated for the disease. In some embodiments, the reference sample is a sample obtained from the subject at an earlier point in time. In some embodiments, the reference sample is obtained from the subject before the subject is treated for the disease.

In various embodiments, the present invention provides a method for assessing and/or determining the risk of developing a disease in a subject; comprising: obtaining a sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; correlating the one or more peptides to one or more proteins, wherein the one or more proteins are listed in Table 15; comparing an amount or level of the one or more proteins in the sample from the subject to an amount or level of the one or more proteins in a reference sample; and determining that the subject has increased risk of developing the disease if the amount or level of the one or more proteins in the sample is changed relative to the reference sample. In some embodiments, the method further comprises measuring an amount or level of one or more proteins in the sample from the subject. In some embodiments, the method further comprises selecting or prescribing a treatment for the subject based on the determination. In some embodiments, the method further comprises treating the subject based on the determination. In some embodiments, the method further comprises administering a treatment to the subject based on the determination. In some embodiments, the method further comprises providing a treatment to the subject based on the determination. In some embodiments, the method further comprises referring the subject to a specialist based on the determination. In some embodiments, the sample is obtained before, during, or after treatment for the disease. In some embodiments the reference sample is obtained from a control subject, wherein the control subject does not have the disease. In some embodiments, the reference sample is obtained from a subject that has been successfully treated for the disease. In some embodiments, the reference sample is a sample obtained from the subject at an earlier point in time. In some embodiments, the reference sample is obtained from the subject before the subject is treated for the disease.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 5. In some embodiments, the mass spectrometry data comprises one or more Q1/Q3 mass value pairs, wherein the Q1/Q3 mass value pairs are correlated to the one or more peptides, and the one or more peptides are correlated to the one or more proteins according to Table 5.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 7. In some embodiments, the mass spectrometry data comprises one or more precursor ions and one or more product ions, wherein the precursor ions and the product ions are correlated to the one or more peptides, and the one or more peptides are correlated to the one or more proteins according to Table 7.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 8. In some embodiments, the mass spectrometry data comprises one or more precursor ions and one or more product ions, wherein the precursor ions and the product ions are correlated to the one or more peptides, and the one or more peptides are correlated to the one or more proteins according to Table 8.

In some embodiments, the mass spectrometry is parallel reaction monitoring (PRM) mass spectrometry. In some embodiments, the one or more peptides are correlated to the one or more proteins according to Table 9. In some embodiments, the mass spectrometry data comprises one or more precursor ions and one or more product ions, wherein the precursor ions and the product ions are correlated to the one or more peptides, and the one or more peptides are correlated to the one or more proteins according to Table 9.

In some embodiments, the mass spectrometry is parallel reaction monitoring (PRM) mass spectrometry. In some embodiments, the one or more peptides are correlated to the one or more proteins according to Table 10. In some embodiments, the mass spectrometry data comprises one or more precursor ions and one or more product ions, wherein the precursor ions and the product ions are correlated to the one or more peptides, and the one or more peptides are correlated to the one or more proteins according to Table 10.

In some embodiments, the mass spectrometry is data dependent acquisition (DDA) mass spectrometry. In some embodiments, the one or more peptides are correlated to the one or more proteins according to Table 11. In some embodiments, the mass spectrometry data comprises one or more precursor ions, wherein the precursor ions are correlated to the one or more peptides, and the one or more peptides are correlated to the one or more proteins according to Table 11.

In some embodiments, the mass spectrometry is data dependent acquisition (DDA) mass spectrometry. In some embodiments, the one or more peptides are correlated to the one or more proteins according to Table 12. In some embodiments, the mass spectrometry data comprises one or more precursor ions and one or more product ions, wherein the precursor ions and the product ions are correlated to the one or more peptides, and the one or more peptides are correlated to the one or more proteins according to Table 12.

In some embodiments, the mass spectrometry is data independent acquisition (DIA) mass spectrometry. In some embodiments, the one or more peptides are correlated to the one or more proteins according to Table 13. In some embodiments, the mass spectrometry data comprises one or more precursor ions and one or more product ions, wherein the precursor ions and the product ions are correlated to the one or more peptides, and the one or more peptides are correlated to the one or more proteins according to Table 13.

In some embodiments, the mass spectrometry is data independent acquisition (DIA) mass spectrometry. In some embodiments, the one or more peptides are correlated to the one or more proteins according to Table 14. In some embodiments, the mass spectrometry data comprises one or more precursor ions and one or more product ions, wherein the precursor ions and the product ions are correlated to the one or more peptides, and the one or more peptides are correlated to the one or more proteins according to Table 14.

In some embodiments, one or more proteases are any one or more of trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof. In some embodiments, the protease is trypsin.

In some embodiments, the method further comprises adding one or more internal standards to the sample. In some embodiments, the internal standard comprises one or more stable isotope-labeled peptide standard, one or more isotopically labeled peptides, one or more isotopically labeled proteins, or any combination thereof.

Kits

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as, for example to treat a disease. Optionally, the kit also contains other useful components, such as, measuring tools, diluents, buffers, pharmaceutical compositions, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example, the components can be dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions, systems, articles of manufacture, and/or protein panels, and the like. The packaging material is constructed by well-known methods, for example to provide a sterile, contaminate-free environment. As used herein, the term “package” refers to a suitable solid matrix or material such as glass, plastic, paper, foil, and the like, capable of holding the individual kit components. The packaging material generally has an external label which indicates the contents and/or purpose of the kit an/or its components.

In various embodiments, the present invention provides a kit for obtaining a protein biomarker signature for a subject, the kit comprising: (a) one or more internal standards suitable for mass spectroscopy; (b) one or more proteases; (c) reagents and instructions for sample processing and preparation; and (d) instructions for using the kit to obtain the protein biomarker signature for the subject. In some embodiments, the internal standard is a stable-isotope labeled peptide standard. In some embodiments, the protease is trypsin. In some embodiments, the protein biomarker signature comprises one or more proteins listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

In various embodiments, the present invention provides a kit for identifying one or more proteins in a sample, the kit comprising: (a) one or more internal standards suitable for mass spectroscopy; (b) one or more proteases; (c) reagents and instructions for sample processing and preparation; and (d) instructions for using the kit to identify one or more proteins in the sample. In some embodiments, the internal standard is a stable-isotope labeled peptide standard. In some embodiments, the protease is trypsin. In some embodiments, the one or more proteins are listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

In various embodiments, the present invention provides a kit for determining the presence of one or more proteins in a sample, the kit comprising: (a) one or more internal standards suitable for mass spectroscopy; (b) one or more proteases; (c) reagents and instructions for sample processing and preparation; and (d) instructions for using the kit to determine the presence of one or more proteins in the sample. In some embodiments, the internal standard is a stable-isotope labeled peptide standard. In some embodiments, the protease is trypsin. In some embodiments, the one or more proteins are listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

In various embodiments, the present invention provides a kit for determining and/or measuring the amount or level of one or more proteins in a sample, the kit comprising: (a) one or more internal standards suitable for mass spectroscopy; (b) one or more proteases; (c) reagents and instructions for sample processing and preparation; and (d) instructions for using the kit to determine and/or measure the amount or level of one or more proteins in the sample. In some embodiments, the internal standard is a stable-isotope labeled peptide standard. In some embodiments, the protease is trypsin. In some embodiments, the one or more proteins are listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integrin alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

In various embodiments, the present invention provides a kit for assessing and/or determining state of health of a subject, the kit comprising: (a) one or more internal standards suitable for mass spectrometry; (b) one or more proteases; (c) reagents and instructions for sample processing and preparation; (d) instructions for using the kit to obtain a protein biomarker signature for the subject and/or instructions for using the kit to determine and/or measure the presence or amount or level of one or more proteins in the sample; (e) one or more reference protein biomarker signatures; and (f) instructions for using the kit to assess and/or determine the state of health of the subject. In some embodiments, the internal standard is a stable-isotope labeled peptide standard. In some embodiments, the protease is trypsin. In some embodiments, the one or more reference biomarker signatures are from one or more subjects having one or more diseases. In some embodiments, the one or more reference biomarker signatures are from one or more healthy subjects.

In various embodiments, the present invention provides a kit for assessing and/or determining state of health of a subject, the kit comprising: (a) one or more internal standards suitable for mass spectrometry; (b) one or more proteases; (c) reagents and instructions for sample processing and preparation; (d) instructions for using the kit to determine and/or measure the presence or amount or level of one or more proteins in the sample; (e) one or more reference protein biomarker signatures; and (f) instructions for using the kit to assess and/or determine the state of health of the subject. In some embodiments, the internal standard is a stable-isotope labeled peptide standard. In some embodiments, the protease is trypsin. In some embodiments, the one or more reference biomarker signatures are from one or more subjects having one or more diseases. In some embodiments, the one or more reference biomarker signatures are from one or more healthy subjects. In some embodiments, the proteins are one or more proteins listed in Table 15.

In various embodiments, the present invention provides a kit for determining, identifying, and/or assessing a risk of developing a disease in a subject, the kit comprising: (a) one or more internal standards suitable for mass spectrometry; (b) one or more proteases; (c) reagents and instructions for sample processing and preparation; (d) instructions for using the kit to obtain a protein biomarker signature for the subject and/or instructions for using the kit to determine and/or measure the presence or amount or level of one or more proteins in the sample; (e) one or more reference protein biomarker signatures; and (f) instructions for using the kit to determine, identify, and/or assess a risk of developing the disease in the subject. In some embodiments, the internal standard is a stable-isotope labeled peptide standard. In some embodiments, the protease is trypsin. In some embodiments, the one or more reference biomarker signatures are from one or more subjects having one or more diseases. In some embodiments, the one or more reference biomarker signatures are from one or more healthy subjects. In some embodiments, the protein biomarker signature comprises one or more proteins listed in Table 15. In some embodiments, the proteins are one or more proteins listed in Table 15.

In various embodiments, the present invention provides a kit for diagnosing a disease in a subject, the kit comprising: (a) one or more internal standards suitable for mass spectrometry; (b) one or more proteases; (c) reagents and instructions for sample processing and preparation; (d) instructions for using the kit to obtain a protein biomarker signature for the subject and/or instructions for using the kit to determine and/or measure the presence or amount or level of one or more proteins in the sample; (e) one or more reference protein biomarker signatures; and (f) instructions for using the kit to diagnose the disease in the subject. In some embodiments, the internal standard is a stable-isotope labeled peptide standard. In some embodiments, the protease is trypsin. In some embodiments, the one or more reference biomarker signatures are from one or more subjects having one or more diseases. In some embodiments, the one or more reference biomarker signatures are from one or more healthy subjects. In some embodiments, the protein biomarker signature comprises one or more proteins listed in Table 15. In some embodiments, the proteins are one or more proteins listed in Table 15.

In some embodiments, one or more proteases are any one or more of trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof. In some embodiments, the protease is trypsin.

In some embodiments, the method further comprises adding one or more internal standards to the sample. In some embodiments, the internal standard comprises one or more stable isotope-labeled peptide standard, one or more isotopically labeled peptides, one or more isotopically labeled proteins, or any combination thereof.

Assays

In various embodiments, the present invention provides an assay for identifying one or more proteins in a sample from a subject, the assay comprising: obtaining the sample from the subject; contacting the the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; and correlating the one or more peptides to one or more proteins so as to identify one or more proteins in the sample, wherein the one or more proteins are listed in Table 15.

In various embodiments, the present invention provides an assay for determining the presence of one or more proteins in a sample from a subject, the assay comprising: obtaining the sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; and correlating the one or more peptides to one or more proteins so as to determine the presence of one or more proteins in the sample, wherein the one or more proteins are listed in Table 15. In some embodiments, the method further comprises measuring and/or detecting the presence of one or more proteins in the sample from the subject.

In various embodiments, the present invention provides an assay for determining the amount or level of one or more proteins in a sample from a subject, the assay comprising: obtaining the sample from the subject; contacting the sample with one or more proteases so as to obtain a digested sample, wherein the digested sample comprises one or more peptides; analyzing the digested sample by mass spectrometry so as to obtain mass spectrometry data; correlating the mass spectrometry data to the one or more peptides; and correlating the one or more peptides to one or more proteins so as to determine the amount or level of one or more proteins in the sample, wherein the one or more proteins are listed in Table 15. In some embodiments, the method further comprises measuring and/or detecting the amount or level of one or more proteins in the sample from the subject.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 5. In some embodiments, the mass spectrometry data comprises one or more Q1/Q3 mass value pairs, wherein the Q1/Q3 mass value pairs are correlated to the one or more peptides, and the one or more peptides are correlated to the one or more proteins according to Table 5.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 7. In some embodiments, the mass spectrometry data comprises one or more precursor ions and one or more product ions, wherein the precursor ions and the product ions are correlated to the one or more peptides, and the one or more peptides are correlated to the one or more proteins according to Table 7.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 8. In some embodiments, the mass spectrometry data comprises one or more precursor ions and one or more product ions, wherein the precursor ions and the product ions are correlated to the one or more peptides, and the one or more peptides are correlated to the one or more proteins according to Table 8.

In some embodiments, the mass spectrometry is parallel reaction monitoring (PRM) mass spectrometry. In some embodiments, the one or more peptides are correlated to the one or more proteins according to Table 9. In some embodiments, the mass spectrometry data comprises one or more precursor ions and one or more product ions, wherein the precursor ions and the product ions are correlated to the one or more peptides, and the one or more peptides are correlated to the one or more proteins according to Table 9.

In some embodiments, the mass spectrometry is parallel reaction monitoring (PRM) mass spectrometry. In some embodiments, the one or more peptides are correlated to the one or more proteins according to Table 10. In some embodiments, the mass spectrometry data comprises one or more precursor ions and one or more product ions, wherein the precursor ions and the product ions are correlated to the one or more peptides, and the one or more peptides are correlated to the one or more proteins according to Table 10.

In some embodiments, the mass spectrometry is data dependent acquisition (DDA) mass spectrometry. In some embodiments, the one or more peptides are correlated to the one or more proteins according to Table 11. In some embodiments, the mass spectrometry data comprises one or more precursor ions, wherein the precursor ions are correlated to the one or more peptides, and the one or more peptides are correlated to the one or more proteins according to Table 11.

In some embodiments, the mass spectrometry is data dependent acquisition (DDA) mass spectrometry. In some embodiments, the one or more peptides are correlated to the one or more proteins according to Table 12. In some embodiments, the mass spectrometry data comprises one or more precursor ions and one or more product ions, wherein the precursor ions and the product ions are correlated to the one or more peptides, and the one or more peptides are correlated to the one or more proteins according to Table 12.

In some embodiments, the mass spectrometry is data independent acquisition (DIA) mass spectrometry. In some embodiments, the one or more peptides are correlated to the one or more proteins according to Table 13. In some embodiments, the mass spectrometry data comprises one or more precursor ions and one or more product ions, wherein the precursor ions and the product ions are correlated to the one or more peptides, and the one or more peptides are correlated to the one or more proteins according to Table 13.

In some embodiments, the mass spectrometry is data independent acquisition (DIA) mass spectrometry. In some embodiments, the one or more peptides are correlated to the one or more proteins according to Table 14. In some embodiments, the mass spectrometry data comprises one or more precursor ions and one or more product ions, wherein the precursor ions and the product ions are correlated to the one or more peptides, and the one or more peptides are correlated to the one or more proteins according to Table 14.

In some embodiments, one or more proteases are any one or more of trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof. In some embodiments, the protease is trypsin.

In some embodiments, the method further comprises adding one or more internal standards to the sample. In some embodiments, the internal standard comprises one or more stable isotope-labeled peptide standard, one or more isotopically labeled peptides, one or more isotopically labeled proteins, or any combination thereof.

Mass Spectrometry

Selected Reaction Monitoring (SRM) and Multiple Reaction Monitoring (MRM)

The terms “SRM” and “MRM” are used interchangeably herein.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 5. In some embodiments, the mass spectrometry data comprises one or more Q1/Q3 mass value pairs, wherein the Q1/Q3 mass value pairs are correlated to the one or more peptides, and the one or more peptides are correlated to the one or more proteins according to Table 5.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 7.

In some embodiments, the mass spectrometry is selected reaction monitoring (SRM) mass spectrometry or multiple reaction monitoring (MRM) mass spectrometry. In some embodiments, the mass spectrometry data comprises one or more precursor ions and one or more product ions, wherein the precursor ions and the product ions are correlated to the one or more peptides, and the one or more peptides are correlated to the one or more proteins according to Table 7. In some embodiments, the precursor ions are observed precursor ions, and the product ions are observed product ions.

In some embodiments, the mass spectrometry is selected reaction monitoring (SRM) mass spectrometry or multiple reaction monitoring (MRM) mass spectrometry. In some embodiments, the one or more peptides are correlated to the one or more proteins according to Table 8.

In some embodiments, the mass spectrometry is selected reaction monitoring (SRM) mass spectrometry or multiple reaction monitoring (MRM) mass spectrometry. In some embodiments, the mass spectrometry data comprises one or more precursor ions and one or more product ions, wherein the precursor ions and the product ions are correlated to the one or more peptides, and the one or more peptides are correlated to the one or more proteins according to Table 8. In some embodiments, the precursor ions are expected precursor ions, and the product ions are expected product ions.

Parallel Reaction Monitoring (PRM)

In some embodiments, the mass spectrometry is parallel reaction monitoring (PRM) mass spectrometry. In some embodiments, the one or more peptides are correlated to the one or more proteins according to Table 9.

In some embodiments, the mass spectrometry is parallel reaction monitoring (PRM) mass spectrometry. In some embodiments, the mass spectrometry data comprises one or more precursor ions and one or more product ions, wherein the precursor ions and the product ions are correlated to the one or more peptides, and the one or more peptides are correlated to the one or more proteins according to Table 9. In some embodiments, the precursor ions are observed precursor ions, and the product ions are observed product ions.

In some embodiments, the mass spectrometry is parallel reaction monitoring (PRM) mass spectrometry. In some embodiments, the one or more peptides are correlated to the one or more proteins according to Table 10.

In some embodiments, the mass spectrometry is parallel reaction monitoring (PRM) mass spectrometry. In some embodiments, the mass spectrometry data comprises one or more precursor ions and one or more product ions, wherein the precursor ions and the product ions are correlated to the one or more peptides, and the one or more peptides are correlated to the one or more proteins according to Table 10. In some embodiments, the precursor ions are expected precursor ions, and the product ions are expected product ions.

Data Dependent Acquisition (DDA)

In some embodiments, the mass spectrometry is data dependent acquisition (DDA) mass spectrometry. In some embodiments, the one or more peptides are correlated to the one or more proteins according to Table 11.

In some embodiments, the mass spectrometry is data dependent acquisition (DDA) mass spectrometry. In some embodiments, the mass spectrometry data comprises one or more precursor ions, wherein the precursor ions are correlated to the one or more peptides, and the one or more peptides are correlated to the one or more proteins according to Table 11. In some embodiments, the precursor ions are observed precursor ions.

In some embodiments, the mass spectrometry is data dependent acquisition (DDA) mass spectrometry. In some embodiments, the one or more peptides are correlated to the one or more proteins according to Table 12.

In some embodiments, the mass spectrometry is data dependent acquisition (DDA) mass spectrometry. In some embodiments, the mass spectrometry data comprises one or more precursor ions and one or more product ions, wherein the precursor ions and the product ions are correlated to the one or more peptides, and the one or more peptides are correlated to the one or more proteins according to Table 12. In some embodiments, the precursor ions are expected precursor ions, and the product ions are expected product ions.

Data Independent Acquisition (DIA)

In some embodiments, the mass spectrometry is data independent acquisition (DIA) mass spectrometry. In some embodiments, the one or more peptides are correlated to the one or more proteins according to Table 13.

In some embodiments, the mass spectrometry is data independent acquisition (DIA) mass spectrometry. In some embodiments, the mass spectrometry data comprises one or more precursor ions and one or more product ions, wherein the precursor ions and the product ions are correlated to the one or more peptides, and the one or more peptides are correlated to the one or more proteins according to Table 13. In some embodiments, the precursor ions are observed precursor ions, and the product ions are observed product ions.

In some embodiments, the mass spectrometry is data independent acquisition (DIA) mass spectrometry. In some embodiments, the one or more peptides are correlated to the one or more proteins according to Table 14.

In some embodiments, the mass spectrometry is data independent acquisition (DIA) mass spectrometry. In some embodiments, the mass spectrometry data comprises one or more precursor ions and one or more product ions, wherein the precursor ions and the product ions are correlated to the one or more peptides, and the one or more peptides are correlated to the one or more proteins according to Table 14. In some embodiments, the precursor ions are expected precursor ions, and the product ions are expected product ions.

In some embodiments, the mass spectrometer is a triple quadrupole mass spectrometer. In some embodiments the mass spectrometer is a Triple-Time Of Flight (Triple-TOF) mass spectrometer configured for SWATH or a Q-Exactive mass spectrometer (Thermo Scientific), or any instrument with sufficiently high scan speed and a quadrupole mass filter to perform data independent acquisition. Examples of triple quadrupole mass spectrometers (TQMS) that can perform MRM/SRM/SIM include but are not limited to: QTRAP® 6500 and 5500 System (Sciex); Triple QTriple Quad 6500 System (Sciex); Agilent 6400 Series Triple Quadrupole LC/MS systems; Thermo Scientific™ TSQ™ Triple Quadrupole system; quadrupole time-of-flight (QTOF) mass spectrometers, or hybrid quadrupole-orbitrap (QOrbitrap) mass spectrometers to carry out the peptides/proteins quantitation. Examples of quadrupole time-of-flight (QTOF) mass spectrometers include but are not limited to: TripleTOF® 6600 or 5600 System (Sciex); X500R QTOF System (Sciex); 6500 Series Accurate-Mass Quadrupole Time-of-Flight (Q-TOF) (Agilent); or Xevo G2-XS QT of Quadrupole Time-of-Flight Mass Spectrometry (Waters). Examples of hybrid quadrupole-orbitrap (QObitrap) mass spectrometers include but are not limited to: Q Exactive™ Hybrid Quadrupole-Orbitrap Mass Spectrometer (the Thermo Scientific); or Orbitrap Fusion™ Tribrid™ (the Thermo Scientific).

In some embodiments, the mass spectrometry technique is tandem mass spectrometry (MS/MS). In some embodiments, the mass spectrometry technique is liquid chromatography-tandom mass spectrometry (LC-MS/MS). In some embodiments, the mass spectrometry technique is liquid chromatography-selected reaction monitoring-mass spectrometry (LC-SRM-MS). In some embodiments, the mass spectrometry technique is liquid chromatography-multiple reaction monitoring-mass spectrometry (LC-MRM-MS). In some embodiments, the mass spectrometry technique is selected reaction monitoring. In some embodiments, the mass spectrometry technique is multiple reaction monitoring. In some embodiments, the mass spectrometry technique is parallel reaction monitoring (PRM). In some embodiments, the mass spectrometry technique is data-independent analysis (DIA). In some embodiments, the mass spectrometry technique is data-dependent analysis (DDA).

In various embodiments, the samples are biological samples or complex biological samples. In exemplary embodiments, the complex samples include, but are not limited to urine, blood, blood fractions, tissues and/or tissue extracts, cells, body fluids, waters, food, terrain and/or synthetic preparations.

In various embodiments, the peptides are derived by proteolysis or chemical cleavage of the polypeptide. In an embodiment, a protease is utilized to cleave the polypeptide into peptides. For example, the protease is trypsin. In additional embodiments, proteases or cleavage agents may be used including but not limited to trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof. In some embodiments, the protease is trypsin.

In various embodiments, the peptides are derived by proteolysis or chemical cleavage of the protein. In an embodiment, a protease is utilized to cleave the protein into peptides. For example, the protease is trypsin. In additional embodiments, proteases or cleavage agents may be used including but not limited to trypsin, chymotrypsin, endoproteinase Lys-C, endoproteinase Asp-N, pepsin, thermolysin, papain, proteinase K, subtilisin, clostripain, exopeptidase, carboxypeptidase, cathepsin C, cyanogen bromide, formic acid, hydroxylamine, or NTCB, or a combination thereof.

In various other embodiments, a list of candidate peptides to be targeted for detection on the analytical instrument is generated by modeling protein cleavage. In exemplary embodiments, a list of candidate peptides to be targeted for detection on the analytical instrument is generated by modeling trypsin digestion of the polypeptide. In some embodiments, the list of candidate peptides is narrowed by eliminating peptides that, for example, cannot be detected on the analytical instrument. In some embodiments, a list of candidate peptides is narrowed by eliminating: a peptide that has not been previously detected on a mass spectrometer, a peptide susceptible to a modification that interferes with accurate quantitation, a miscleaved peptide comprising an internal protease recognition site, a peptide with relatively inaccessible ends evidenced by the presence of miscleaved peptides, a peptide that is not unique to the sequence of the protein of interest, a peptide not present in the mature protein, or a combination thereof.

In an embodiment, the detection of a peptide is improved by changing the conditions for fragmenting that peptide prior to detecting a multiplicity of the peptides with the mass spectrometer. In exemplary embodiments, the fragmentation condition is the collision energy.

In various other embodiments, the method further comprises adding a stable isotope-labeled peptide to the sample prior to mass spectrometry. In some embodiments, the absolute amount of a peptide in the sample is determined by comparing the MS signals of natural and stable isotope-labeled peptides.

In other embodiments, the comprehensive list of candidate peptides is narrowed by eliminating peptides. In other embodiments, conventional criteria are used to eliminate peptides from the comprehensive list of candidate peptides by eliminating peptides that: (i) were never detected by MS on any instrument, (ii) are not unique to the sequence of the protein of interest, (iii) are not located within the mature protein, (iv) contain amino acid residues such as methionine, cysteine, and/or asparagine that are subjected to posttranslational modifications that interfere with accurate quantitation by mass spectrometry, (v) are miscleaved or partially cleaved, (vi) are post-translationally modified in vivo, (vii) and/or a combination thereof.

In various other embodiments, transitions for each peptide with high and reproducible peak intensities are identified. In other embodiments, the collision energy for each transition is optimized. In other embodiments, mass spectrometry comprises selected reaction monitoring (SRM), or multiple reaction monitoring (MRM). In other embodiments, SRM or MRM is performed on a triple quadrapole mass spectrometer. In other embodiments, the peptides uniquely associated with the polypeptide of interest are those with high correlations, strong signals, high signal/noise and/or sequences unique to the protein of interest.

Selected-ion monitoring (SIM) or selected reaction monitoring (SRM) or multiple reaction monitoring (MRM) provide the simplest method set up and the most selective and sensitive quantification. SRM/MRM/SIM is a method used in tandem mass spectrometry in which an ion of a particular mass is selected in the first stage of a tandem mass spectrometer and an ion product of a fragmentation reaction of the precursor ion is selected in the second mass spectrometer stage for detection. Examples of triple quadrupole mass spectrometers (TQMS) that can perform MRM/SRM/SIM include but are not limited to: QTRAP® 6500 and 5500 System (Sciex); Triple QTriple Quad 6500 System (Sciex); Agilent 6400 Series Triple Quadrupole LC/MS systems; or Thermo Scientific™ TSQ™ Triple Quadrupole system.

In addition to MRM, the choice of peptides can also be quantified through Parallel-Reaction Monitoring (PRM). Parallel reaction monitoring (PRM) is the application of SRM with parallel detection of all transitions in a single analysis using a high resolution mass spectrometer. PRM provides high selectivity, high sensitivity and high-throughput to quantify selected peptide (Q1), hence quantify proteins. Again, multiple peptides can be specifically selected for each protein. PRM methodology uses the quadrupole of a mass spectrometer to isolate a target precursor ion, fragments the targeted precursor ion in the collision cell, and then detects the resulting product ions in the Orbitrap mass analyzer. Quantification is carried out after data acquisition by extracting one or more fragment ions with 5-10 ppm mass windows. PRM uses a quadrupole time-of-flight (QTOF) or hybrid quadrupole-orbitrap (QOrbitrap) mass spectrometer to carry out the peptides/proteins quantitation. Examples of QTOF include but are not limited to: TripleTOF® 6600 or 5600 System (Sciex); X500R QTOF System (Sciex); 6500 Series Accurate-Mass Quadrupole Time-of-Flight (Q-TOF) (Agilent); or Xevo G2-XS QT of Quadrupole Time-of-Flight Mass Spectrometry (Waters). Examples of QObitrap include but are not limited to: Q Exactive™ Hybrid Quadrupole-Orbitrap Mass Spectrometer (the Thermo Scientific); or Orbitrap Fusion™ Tribrid™ (the Thermo Scientific).

Non-limiting advantages of PRM include elimination of most interferences, provides more accuracy and attomole-level limits of detection and quantification, enables the confident confirmation of the peptide identity with spectral library matching, reduces assay development time since no target transitions need to be preselected, ensures UHPLC-compatible data acquisition speeds with spectrum multiplexing and advanced signal processing.

In various other embodiments, stable isotope-labeled peptide standards for absolute quantification are used. In other embodiments, the peptide labeled with a stable isotope is used as an internal standard to obtain absolute quantification of the polypeptide of interest. In other embodiments, the peptides are quantified and then the amount of the parent protein present is inferred before digesting the sample with trypsin. In other embodiments, MS responses are used to determine an upper limit of quantification (ULOQ) and a lower limit of quantification (LLOQ).

In various embodiments, the MS data comprises raw MS data obtained from a mass spectrometer and/or processed MS data in which peptides and their fragments (e.g., transitions and MS peaks) are already identified, analyzed and/or quantified. In various embodiments, the MS data is Selective Reaction Monitoring (SRM) data or Parallel-Reaction Monitoring (PRM) data and/or Multiple Reaction Monitoring (MRM) data. In various embodiments, the MS data is Shotgun CID MS data, Original DIA MS Data, MSE MS data, p2CID MS Data, PAcIFIC MS Data, AIF MS Data, XDLA MS Data, SWATH MS data, or FT-ARM MS Data, or a combination thereof.

In some embodiments, acquiring MS data does not require operating a mass spectrometer. For examples, MS data can be acquired from MS experiments run previously and/or MS databases. In some embodiments, previously acquired SWATH MS data can be queried with a more comprehensive library to identify additional MS peaks derived from different and macromolecules.

In various embodiments, acquiring MS data comprises operating a TripleTOF mass spectrometer, a triple quadrupole mass spectrometer, a liquid chromatography-mass spectrometry (LC-MS) system, a gas chromatography-mass spectrometry (GC-MS) system, or a tandem mass spectrometry (MS/MS) system, a dual time-of-flight (TOF-TOF) mass spectrometer, or a combination thereof.

In various embodiments, acquiring MS data comprises operating a mass spectrometer. Examples of the mass spectrometer include but are not limited to high-resolution instruments such as Triple-TOF, Orbitrap, Fourier transform, and tandem time-of-flight (TOF/TOF) mass spectrometers; and high-sensitivity instruments such as triple quadrupole, ion trap, quadrupole TOF (QTOF), and Q trap mass spectrometers; and their hybrid and/or combination. High-resolution instruments are used to maximize the detection of peptides with minute mass-to-charge ratio (m/z) differences. Conversely, because targeted proteomics emphasize sensitivity and throughput, high-sensitivity instruments are used. In some embodiments, the mass spectrometer is a TripleTOF mass spectrometer. In some embodiments, the mass spectrometer is a triple quadrupole mass spectrometer.

In various embodiments, the MS data is collected by a targeted acquisition method. Examples of the targeted acquisition method include but are not limited to Selective Reaction Monitoring (SRM) and/or Multiple Reaction Monitoring (MRM) methods. In various embodiments, acquiring MS data comprises acquiring Selective Reaction Monitoring (SRM) data and/or Multiple Reaction Monitoring (MRM) data.

In various embodiments, the MS data is collected by a data independent acquisition (DIA) method. In various embodiments, the MS data is collected by data dependent acquisition (DDA) method.

Non-limiting examples of mass spectrometry techniques include collision-induced dissociation (CID), higher-energy collisional dissociation (HCD), electron-transfer dissociation (ETD), etc.

In various embodiments, the MS data is collected by a data independent acquisition method. Examples of the independent acquisition (DIA) method including but not limited to Shotgun CID (see. e.g., Purvine et al. 2003), Original DIA (see e.g., Venable et al. 2004), MS^{E} (see e.g., Silva et al. 2005), p2CID (see e.g., Ramos et al. 2006), PAcIFIC (see e.g., Panchaud et al. 2009), AIF (see e.g., Geiger et al. 2010), XDLA (see e.g., Carvalho et al. 2010), SWATH (see e.g., Gillet et al. 2012), and FT-ARM (see e.g., Weisbrod et al. 2012). More information can be found in, for example, Chapman et al. (Multiplexed and data-independent tandem mass spectrometry for global proteome profiling, Mass Spectrom Rev. 2014 November-December; 33(6):452-70). In various embodiments, acquiring MS data comprises acquiring Shotgun CID MS data, Original DIA MS Data, MS^{E} MS data, p2CID MS Data, PAcIFIC MS Data, AIF MS Data, XDLA MS Data, SWATH MS data, or FT-ARM MS Data, or a combination thereof. In certain embodiments, acquiring MS data comprises acquiring MS data comprises acquiring SWATH MS data.

In various embodiments, the sample is food, water, cheek swab, blood, serum, plasma, urine, saliva, semen, cell sample, tissue sample, or tumor sample, or a combination thereof. In some embodiments the sample is plasma, serum, cerebrospinal fluid (CSF), a tissue extract, or a biopsy sample.

Abbreviations: MS, Mass Spectrometry; LC-MS/MS, liquid chromatography-tandem mass spectrometry; LC-SRM-MS, liquid chromatography selected reaction monitoring mass spectrometry; OGS, N-octyl glucoside; MMTS, Methyl methanethiosulfonate; TCEP, Tris-(2-carboxyethyl)-phosphine; FA, Formic acid; TPCK treated trypsin, Trypsin treated with L-(tosylamido-2-phenyl) ethyl chloromethyl ketone; β-Gal, β-galactosidase; CV %. Coefficient of variation; LC-SRM-MS, Liquid chromatography-selected reaction monitoring mass spectrometry; SIL peptide, Stable Isotope-Labeled Peptide; DIA-MS, Data Independent acquisition mass spectrometry; SPE, solid phase extraction; IQ panel, internal quality panel.

The automated proteomics sample preparation workflow was first developed and tested with a robust LC-SRM-MS acquisition method for albumin (serum albumin), the highest abundance plasma protein, and β-galactosidase (β-gal), an exogenous protein used for quality control. For compatibility with an automated workflow, we selected reagents that have negligible non-specific side reactions, are stable in ambient light, are LC-MS/MS friendly, and can be stored as frozen aliquots. To monitor consistency (to detect and normalize for any variability arising from the SRM analysis), we added intact β-gal protein and a mixture of stable-isotope labeled (SIL) peptide standards (SIL albumin and β-gal peptides) before the reduction and alkylation reactions.

As a preliminary test, we automated the reagent addition and mixing steps for a digestion of 5 μL plasma samples with a 16-hour off-deck trypsin incubation followed by manual solid phase extraction (SPE). The variability of the peak area ratios (native/SIL peptide signal) for β-gal and albumin were an unacceptably high 25.6% CV and 27.1% CV, respectively (set B in Table 2a). The variability (coefficient of variation (CVs)) of the peak area ratios (native/SIL peptide signal) for β-gal and albumin were an unacceptably high 25.6% CV and 27.1% CV, respectively (set B in Table 2a).

To determine which steps in this procedure contributed substantially to technical variation, we pooled samples and then re-aliquoted at different stages in the process (Table 2a and Table 2b). When samples were pooled and re-aliquoted immediately before LC-MS/MS (set D), the average % CV was 4.1% for β-gal and 3.3% for albumin, indicating that the final LC-MS/MS step is highly reproducible. Pooling and re-aliquoting before the SPE desalting step (set C) resulted in an average % CV of 14.8% for β-gal and 18.2% for albumin, indicating that SPE desalting accounted for nearly half of the variability. We tested two online desalting methods as replacements for SPE. The % CVs for β-gal and albumin dropped to 10.0% and 8.0%, respectively, for simple online diversion (set E) and to 10.0% and 7.0%, respectively, with a trap column (set F).

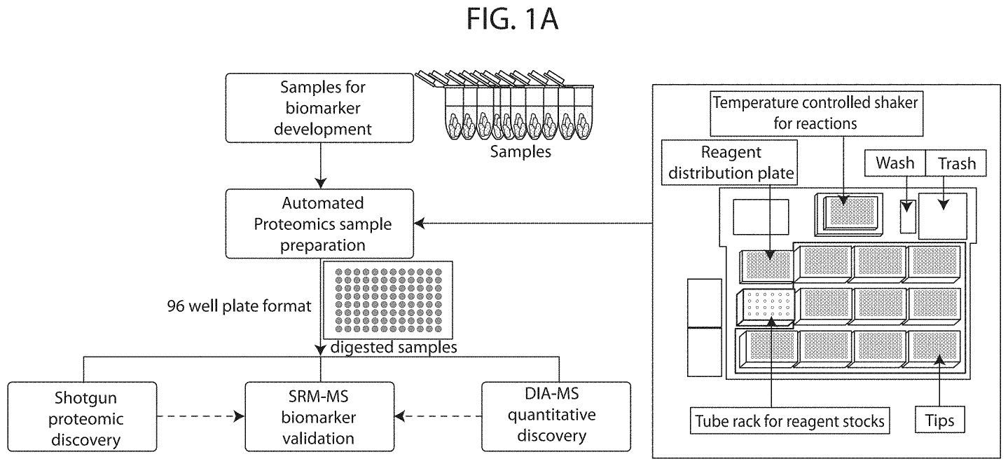

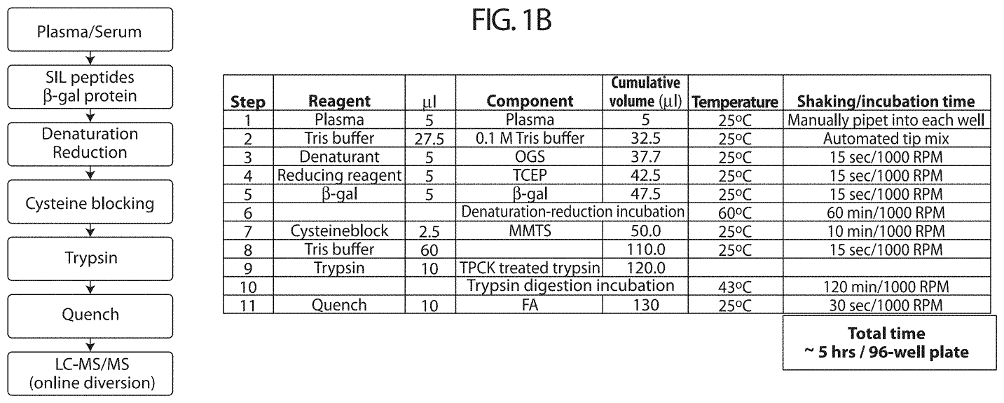

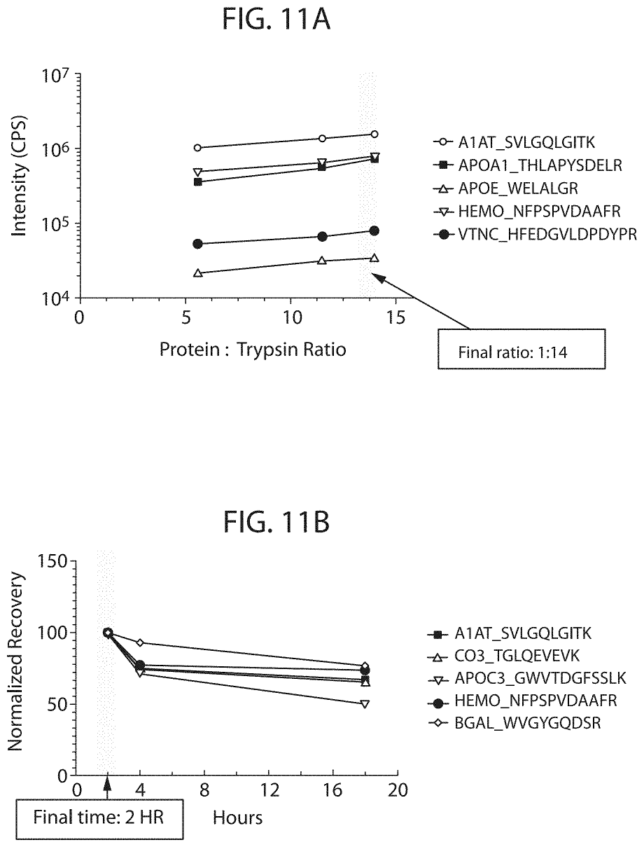

Optimizing Trypsin Digestion Conditions. In an effort to save time and simplify the procedure, we optimized the digestion workflow by testing various reagents and concentrations, incubation times, and trypsin to substrate ratios. In the revised process, the trypsin to plasma protein ratio was optimized for a two-hour incubation at 37° C. For the initial optimization tests, the digestion plate was removed from the automated workstation, sealed, and shaken at 1000 rpm for two benchtop incubation steps: (1) denaturation and reduction for 1 hour at 60° C. and (2) trypsin digestion at 37° C. After these incubations, the plate was centrifuged briefly and then returned to the automated workstation for additional processing. The trypsin/substrate ration was varied by digesting 2, 4, or 5 μL plasma with a constant amount of trypsin (25 μg) for 2 hours. Assuming a plasma protein concentration of 70 mg/mL, this corresponds to trypsin/substrate (w/w) ratios of 1:5.6, 1:11.2, and 1:14. All three ratios yielded similar results for albumin and β-gal (FIG. 11A), so we chose to use 5 μL of plasma as it allowed us to have more material for additional downstream experiments. Next, the digestion time was varied by quenching reactions with formic acid (FA) and the stroing them at −80° C. until all time points were collected. The signal intensity of all five albumin and β-gal peptides was highest after a 2 hour digestion (FIG. 11B). By automating this streamlined procedure on a Biomek NV Workstation (set G) with a 2 hour digestion at a 1:14 (w/w) trypsin/substrate ratio, the average % CV for the complete process was further reduced to 2.6% for albumin and 5.5% for β-gal. FIG. 1A-FIG. 1B illustrates the schema and protocol for the final automated sample preparation workflow.

Reproducibility of the Automated Digestion Workflow. To validate the automated proteomics sample preparation workflow, we first evaluated reproducibility across multiple proteins and multiple days for serum albumin, β-gal and five additional well-studied plasma biomarkers: complement C3, alpha-1-antitrypsin, alpha-1-acid glycoprotein 1, hemopexin, and apolipoprotein (Table 3). We processed five replicate samples (pooled normal human serum and pooled normal human plasma) on five different days. Intra-day CVs were calculated by comparing the results of five replicate sample preparations run on the same day; mean intra-day CVs for the seven proteins ranged from 5.5-8.9% in serum and from 3.9-6.2% for plasma (or from 3.9-7.2% for plasma) (Table 1, top). Inter-day CVs were calculated by comparing each replicate run on separate days; mean inter-day CVs for the seven proteins ranged between 5.8-10.6% in serum and between 3.9-6.0% in plasma (Table 1 and Tables 4a-4c). Total CVs were calculated from the square root of the sum of squares of the mean intra- and inter-day CVs; these ranged from 9.2-12.3% for serum and 6.4-8.3% for pooled plasma (or 6.4-9.4% for pooled plasma) (Table 1, bottom). All of the total CVs were less than 20%, satisfying the best practice acceptance criterion for assay validation of LC-MS/MS protein quantitation¹⁵.

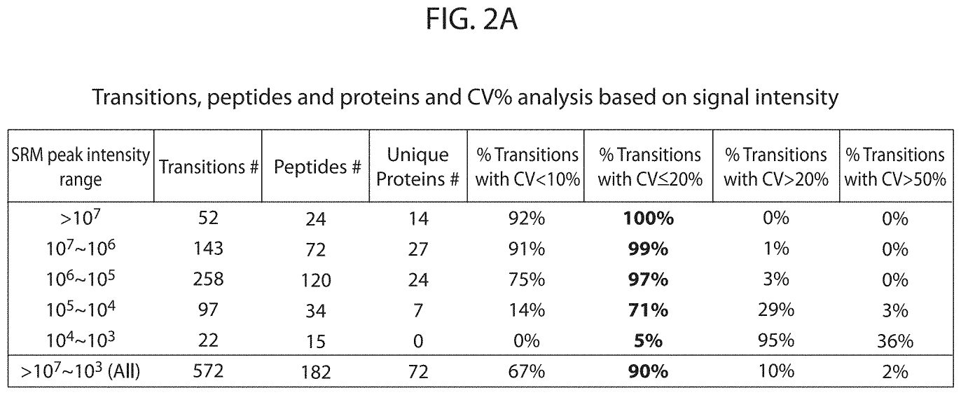

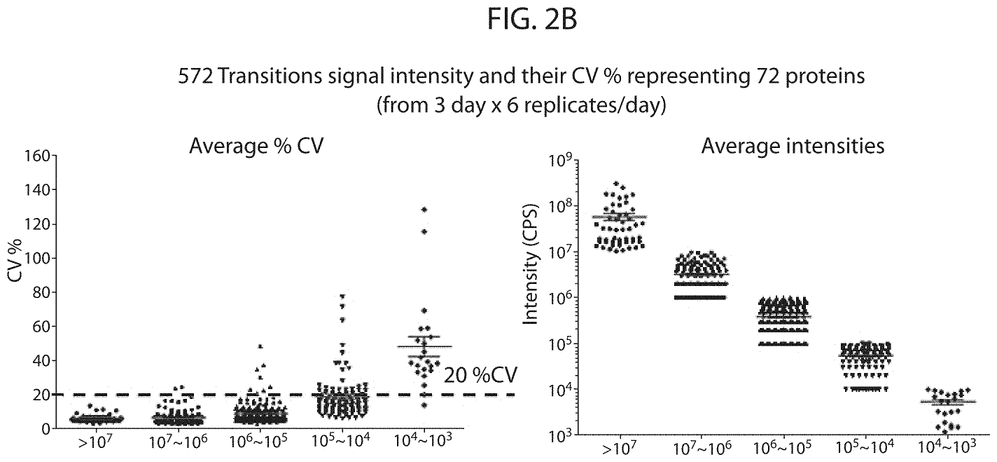

Reproducibility in a Highly Multiplexed SRM Assay. We next validated the general applicability of the optimized workflow with an expanded LC-SRM-MS method targeting 72 high-abundance plasma proteins (Table 5). This internal quality (IQ) SRM-panel monitors a total of 572 transitions from 182 peptides in a single scheduled 50-minute LC run (Table 5). 90% of the IQ transitions had total CVs below 20%, as measured from six replicate automated sample preparations on three independent days (FIG. 2A). The SRM signal intensities ranged from 1×10³ to >1×10⁷ cps (FIG. 2B, right panel), and the transitions with greater intensity had higher precision (FIG. 2B, left panel).

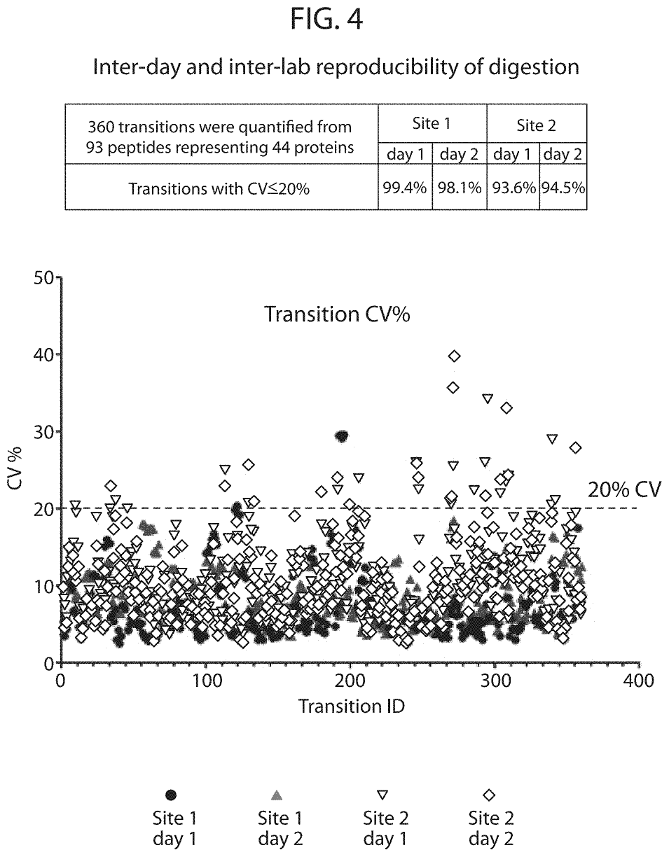

Reproducibility between laboratories. Reproducibility between laboratories has emerged as a major concern impeding wider acceptance of quantitative MS assays¹⁶. Eliminating the variability associated with manual sample processing in different environments could improve transferability of proteomic LC-MS/MS assays. As part of validation, we implemented an identical automated proteomics sample preparation workflow for plasma in two different laboratories (CSMC and SCIEX), followed by LC-SRM-MS measurement of 93 peptides from 44 proteins on a QTRAP® 6500 system with either micro flow LC (SCIEX) or high flow LC (CSMC). Overall, more than 93% of the peptides had CVs below 20% on at both sites on two separate days (FIG. 4)¹⁷⁻¹⁹.

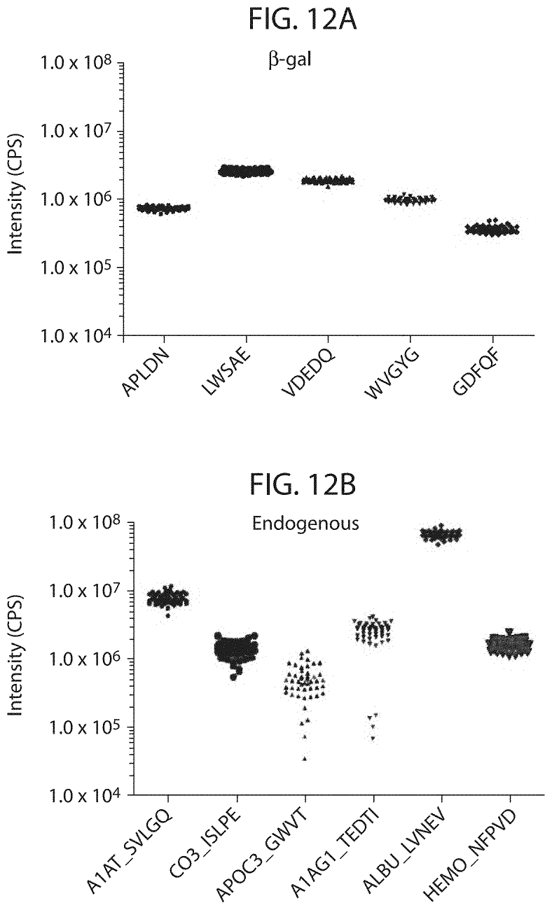

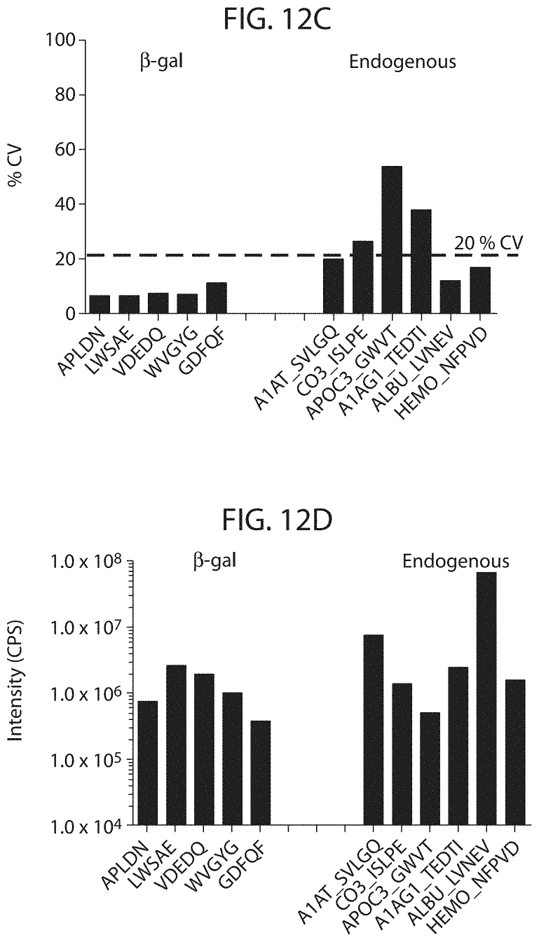

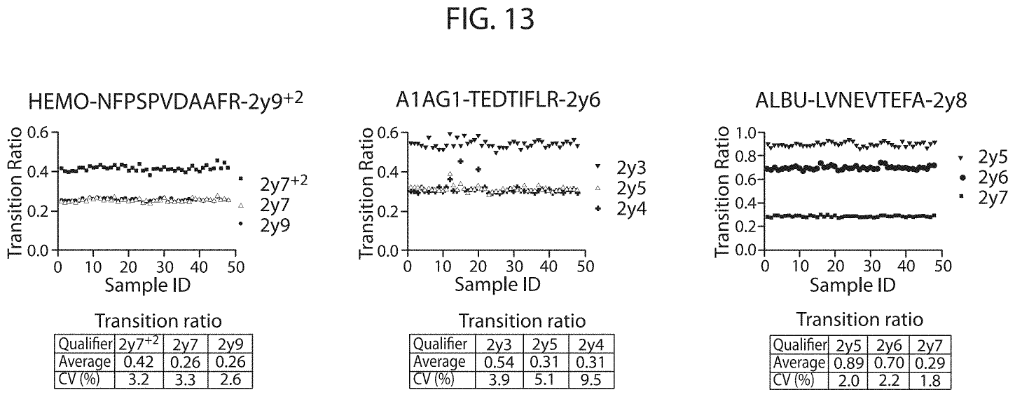

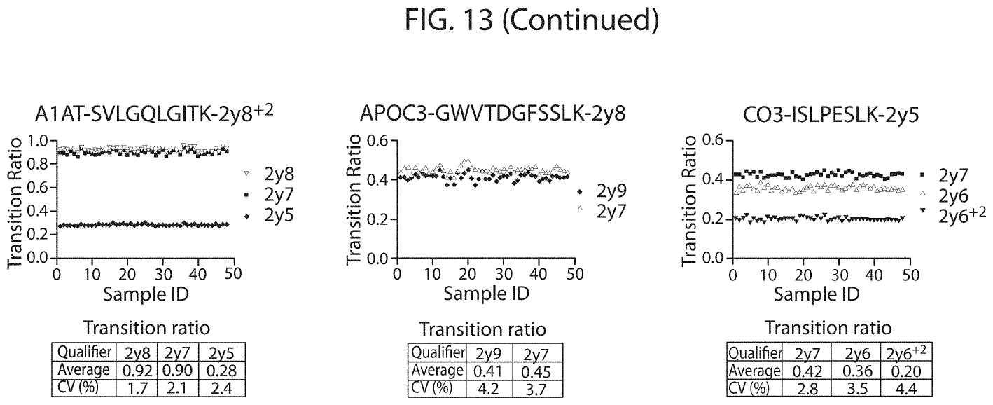

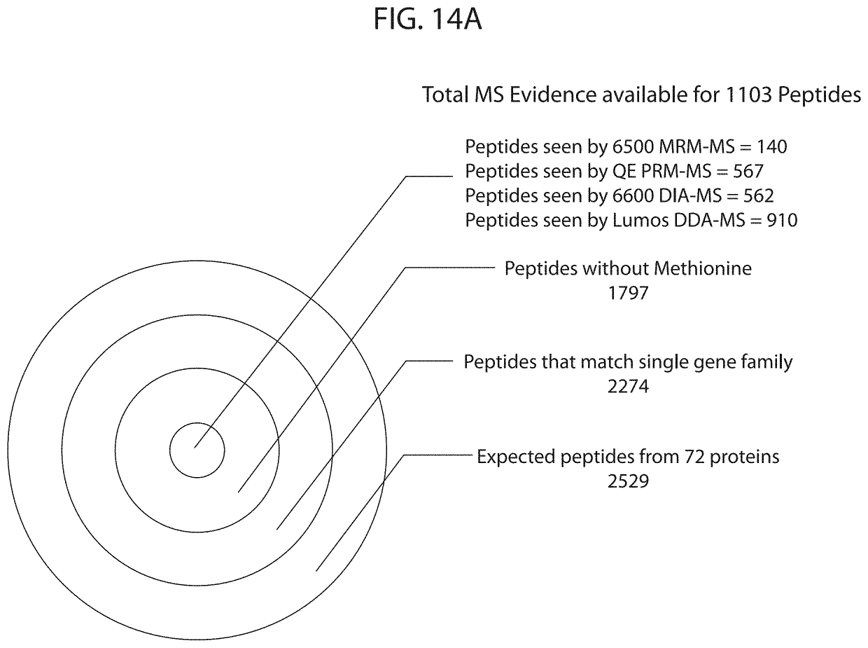

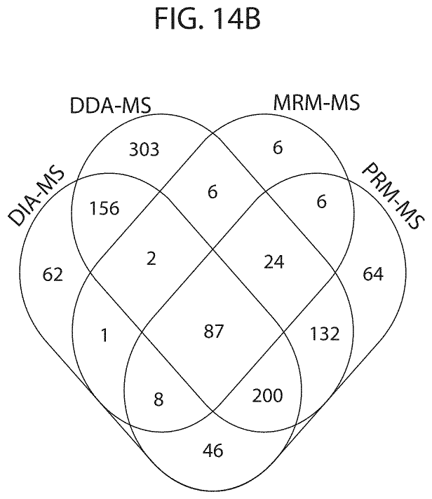

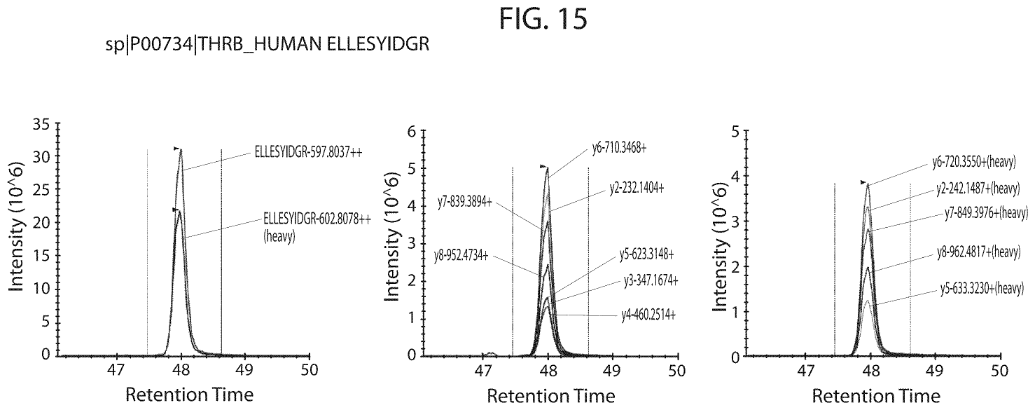

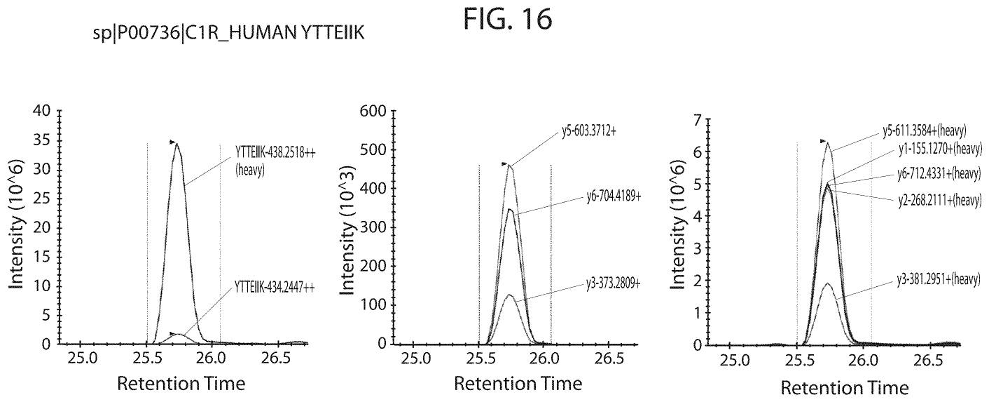

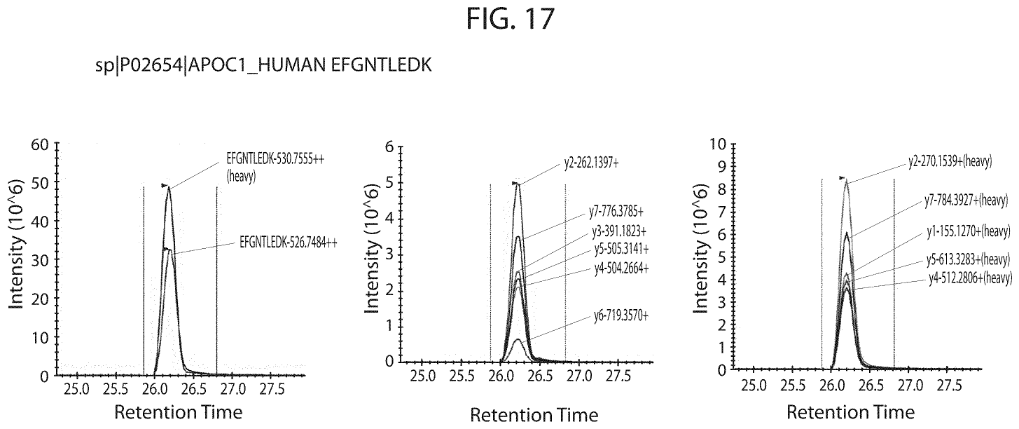

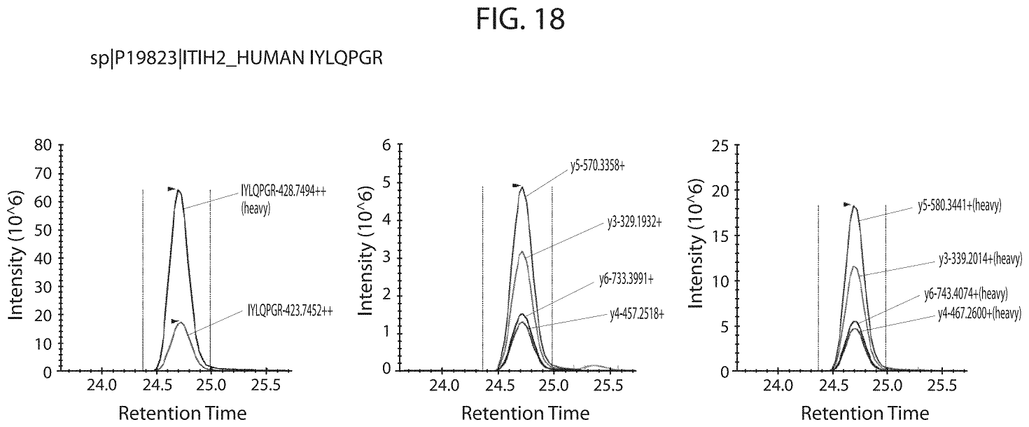

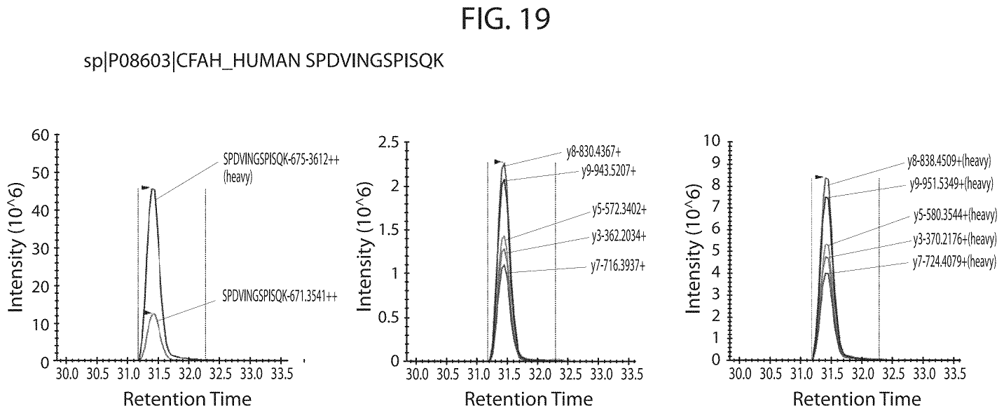

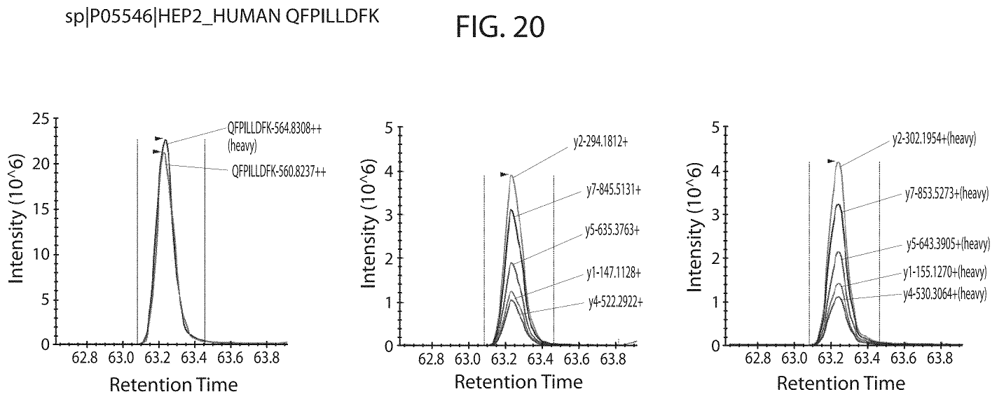

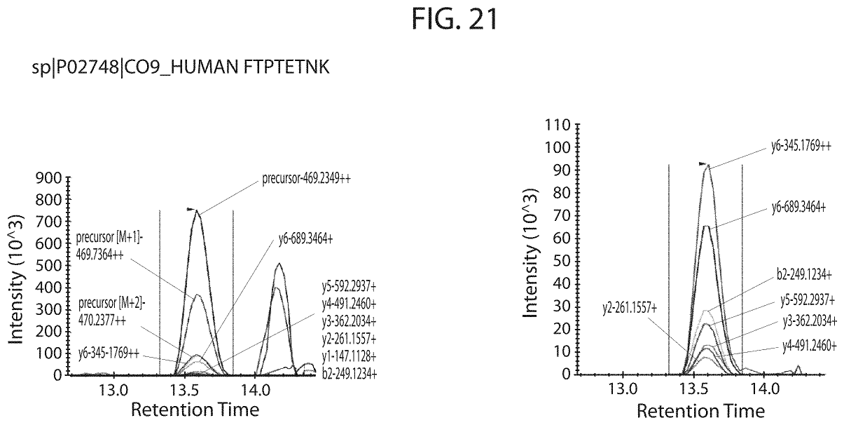

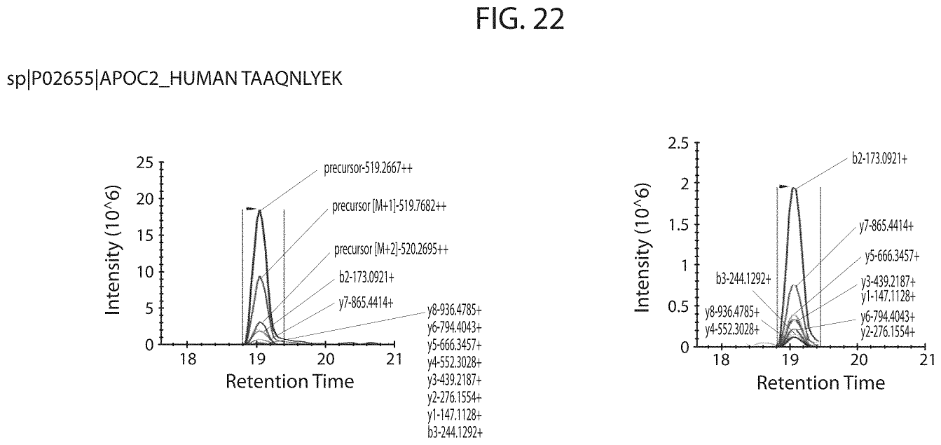

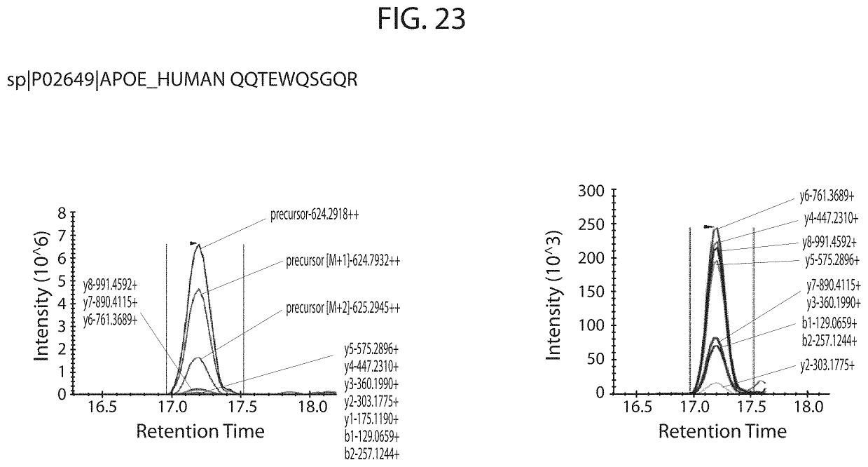

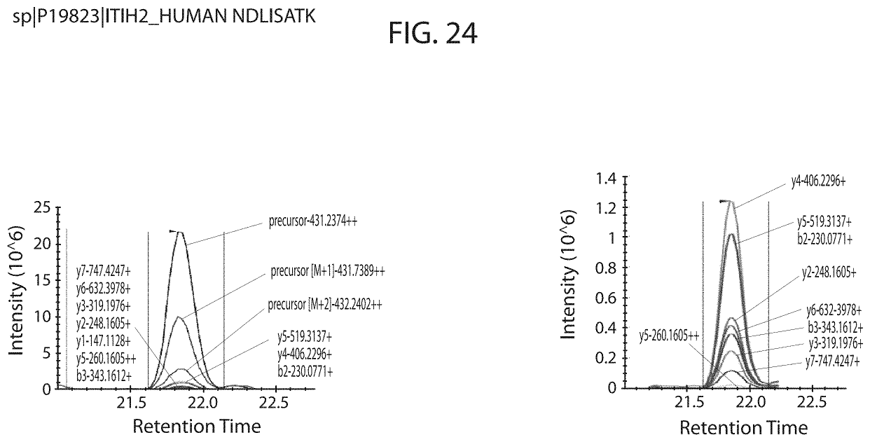

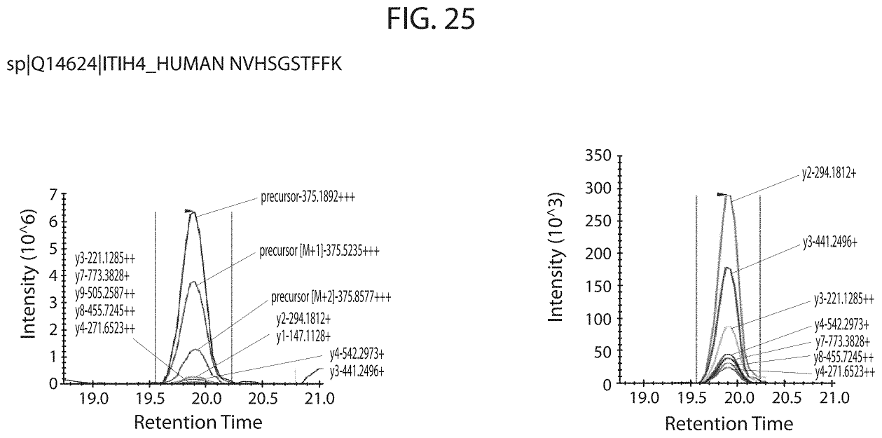

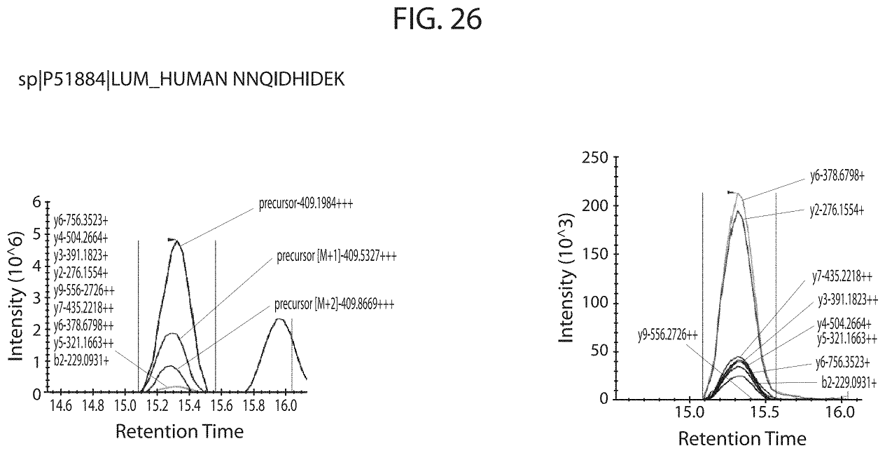

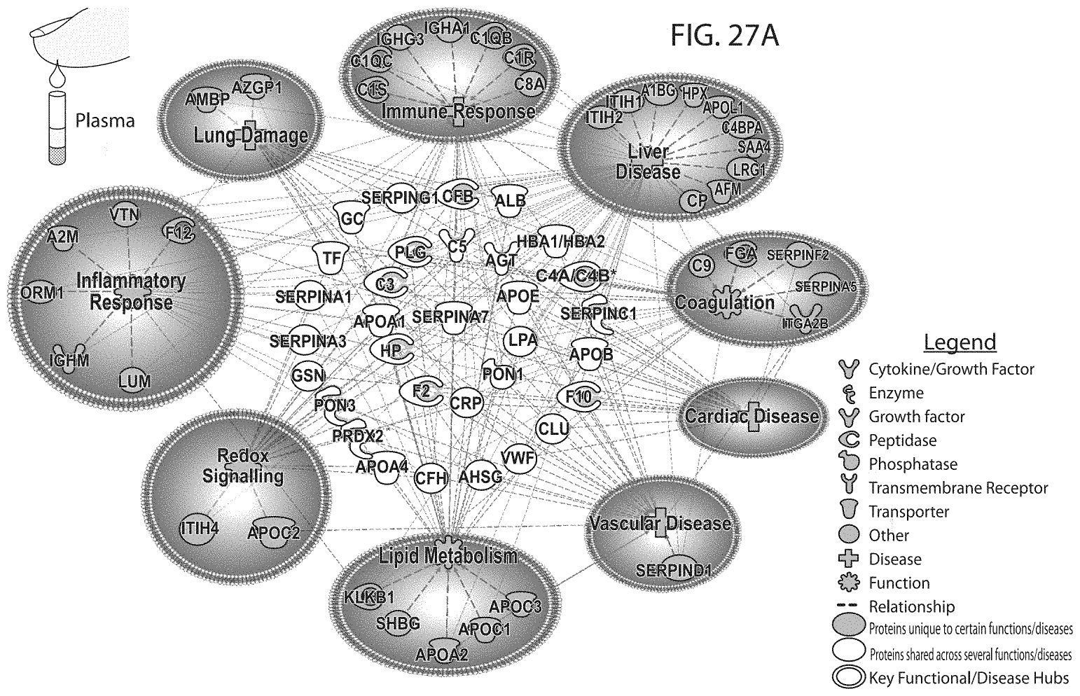

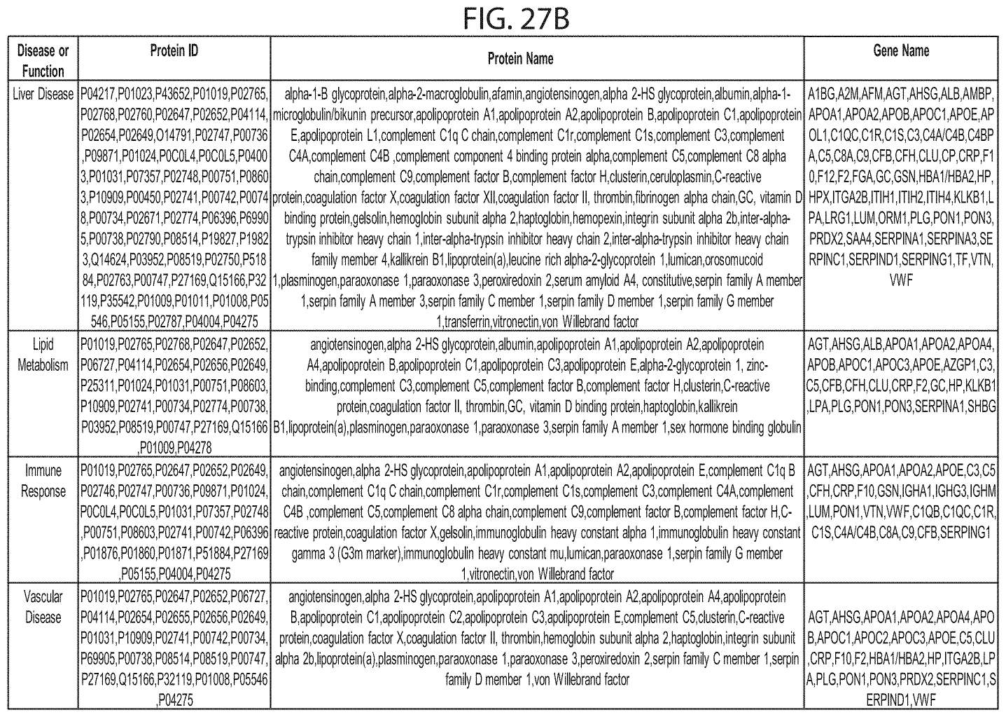

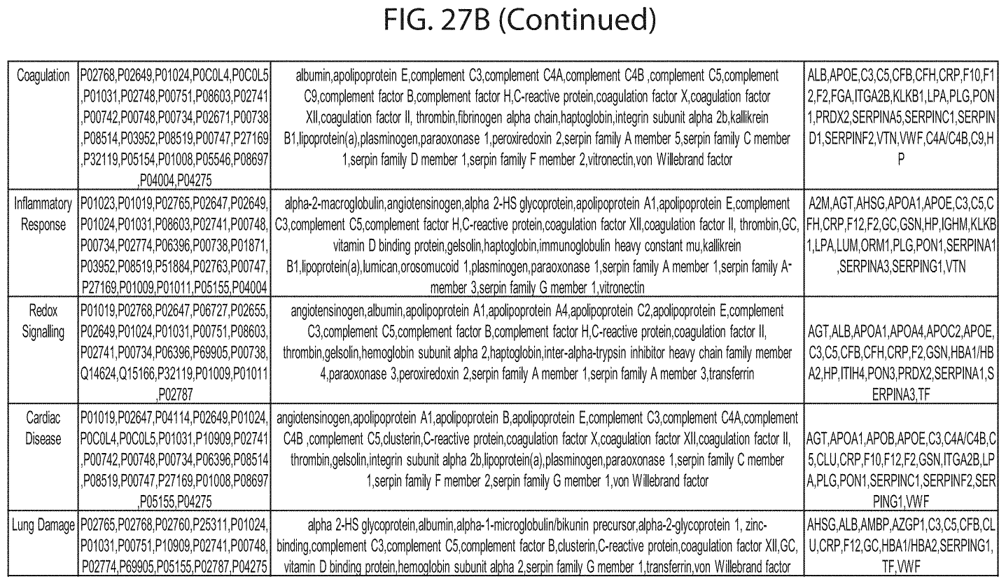

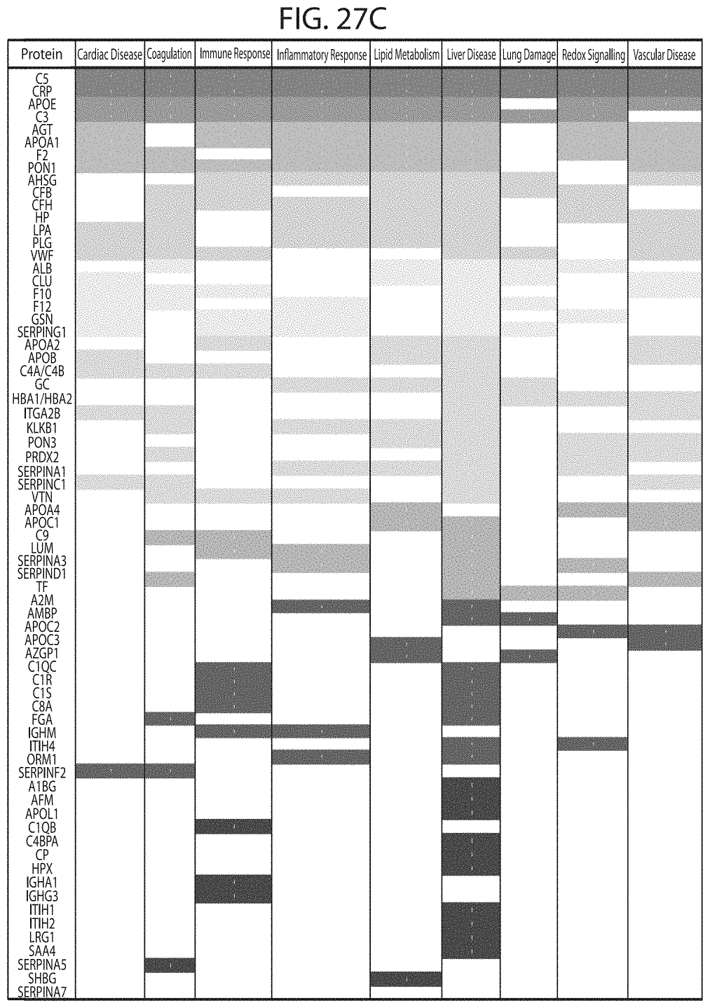

Reproducibility Across Plasma Samples. Even with an automated workflow, plasma samples from different individuals might vary in ways that effect accurate quantification. To evaluate matrix effects on the accuracy of MS quantification, the automated workflow as tested on 48 human plasma samples (24 individuals with known coronary artery disease and 24 age and gender matched controls). All samples were processed on a single 96-well plate, and the resulting peptides were analyzed with the highly multiplexed SRM assay. In this analysis, data for peptides derived from β-gal that had been spiked into each sample before processing show the technical variation, which includes effects of the sample matrix on sample processing and analysis (FIG. 12A). Five β-gal peptides had CVs ranging from 6.7 to 10.5% (FIG. 12C). Variations in the amounts of peptides derived from endogenous plasma proteins include a combination of technical variation and biological variation in protein levels (FIG. 12B). Exemplary proteotypic peptides derived from endogenous plasma proteins had CVs ranging from 12.0 to 54.1%, consistent with the expected biological variation between individuals (FIG. 12C). These results demonstrate that the analytical variance is low enough to enable measurements of the biological variation.

Analytical Selectivity. Despite the highly selective nature of SRM, inference is possible due to the complex and variable composition of plasma. To test for interference, three to five transitions were monitored for each peptide. The best transition, generally a y ion with a high signal and a symmetrical peak, was designated as the quantifier. Other transitions were used for qualification. The relative signal intensities of the qualifier and quantifier transitions should be constant unless there are interferences. Indeed, for six exemplary peptides measured in 48 plasma samples, the qualifier/quantifier ratio for at least one qualifier transition had a CV of less than 4%. Furthermore, all of the qualifier/quantifier ratios had CVs lower than the 20% standard set by the Clinical and Laboratory Standards Institute. Thus, results for the processed plasma samples were not compromised by interferences.

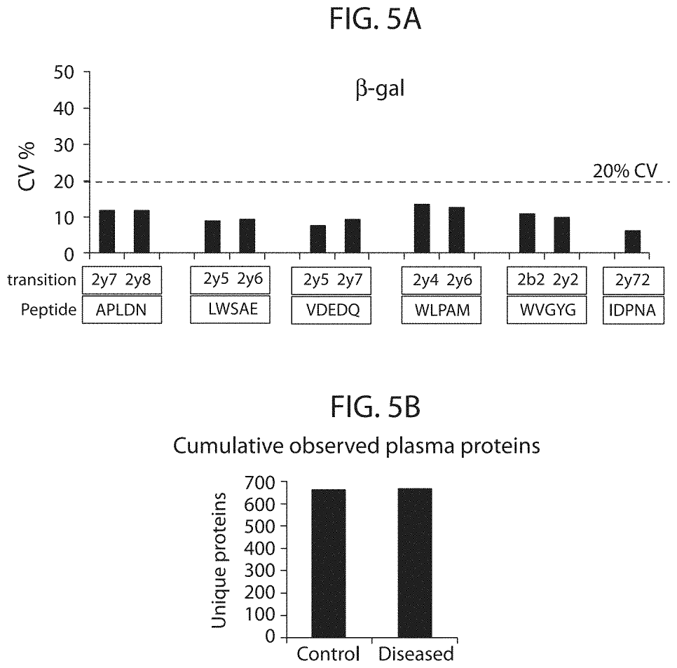

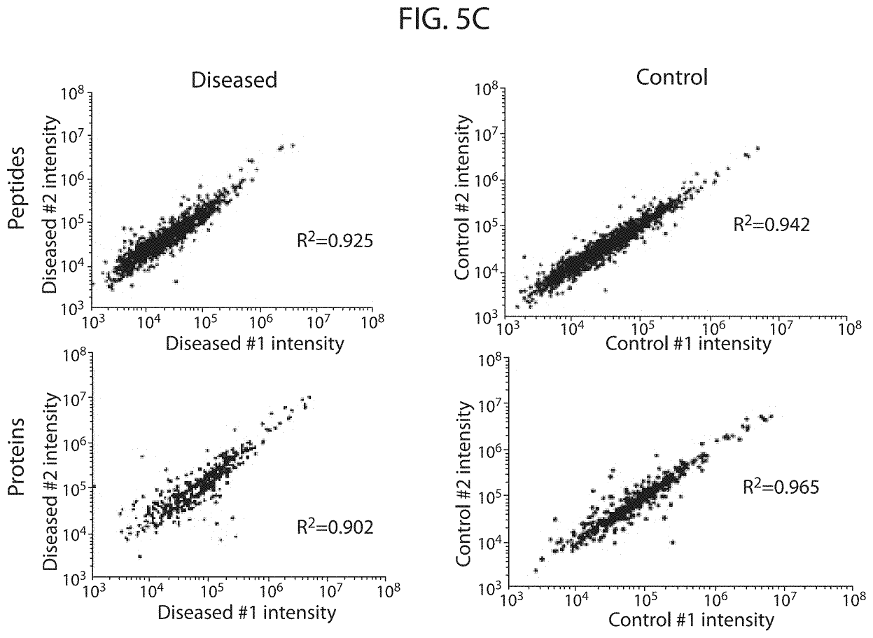

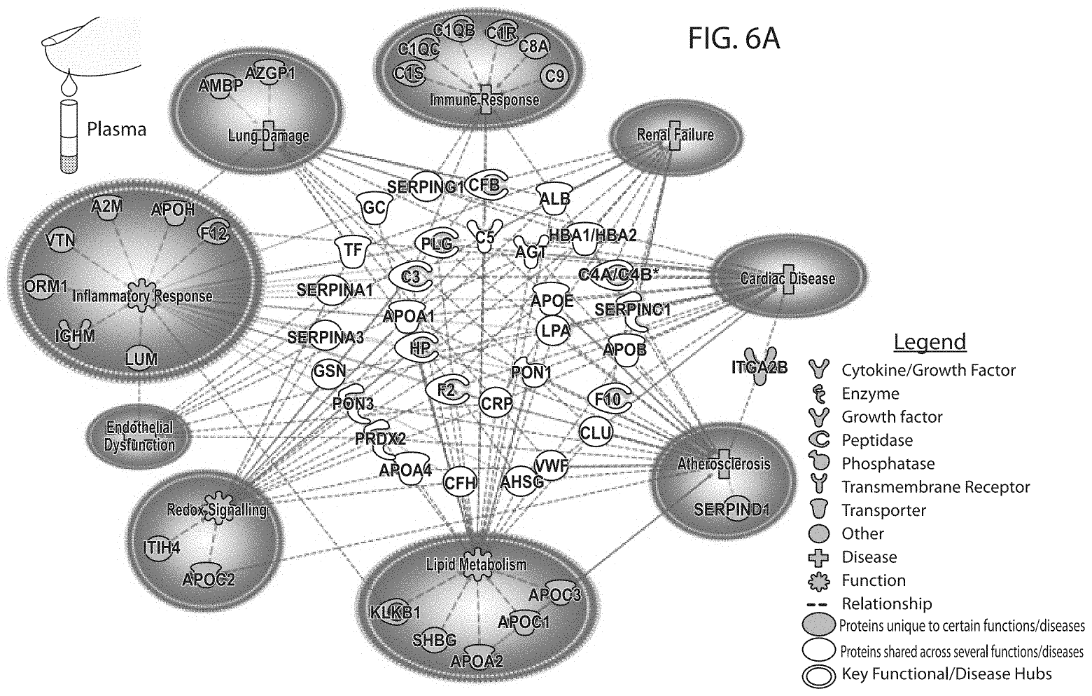

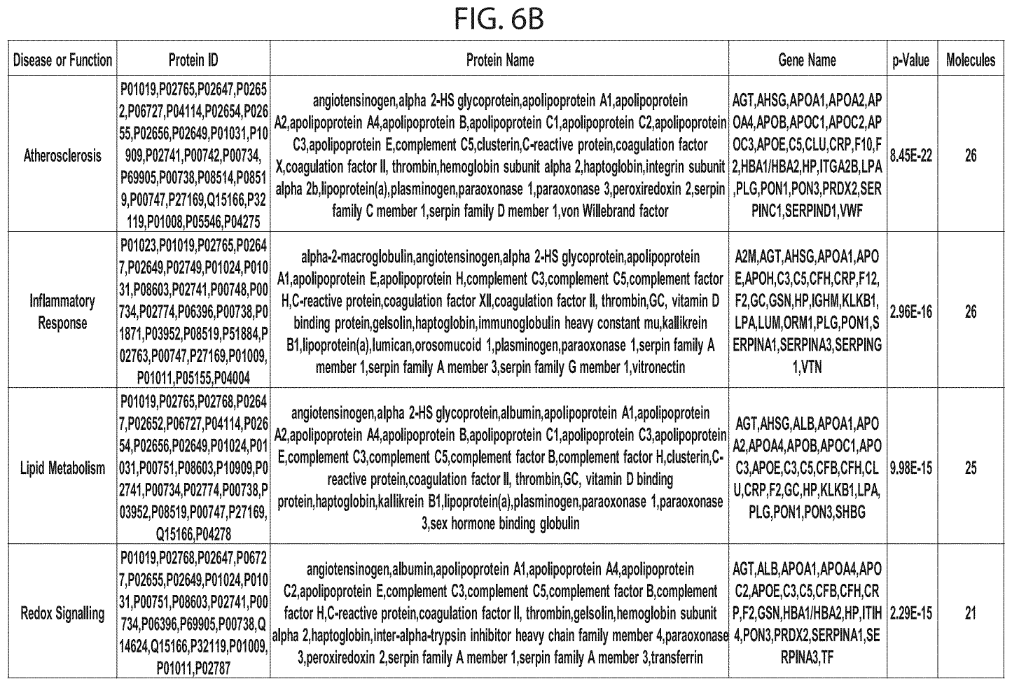

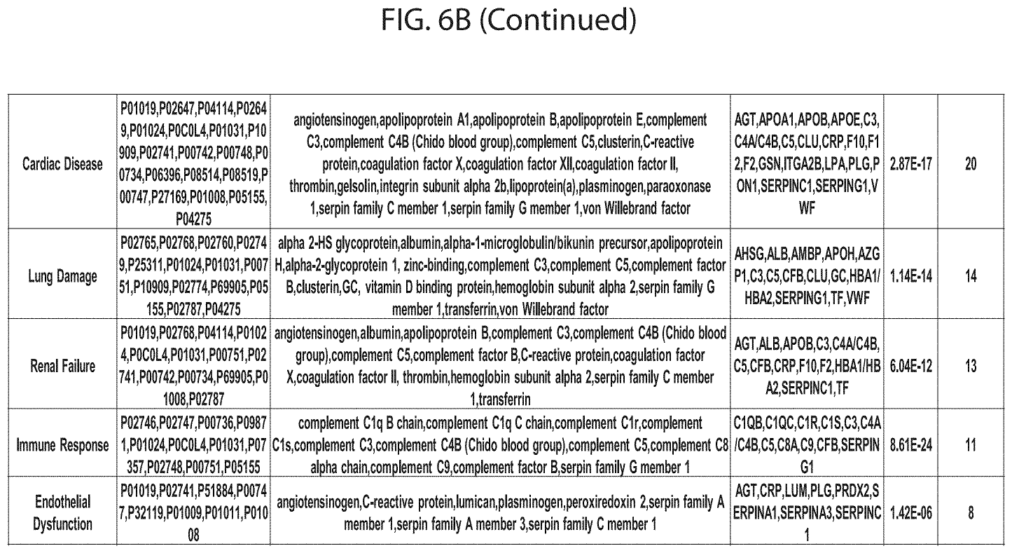

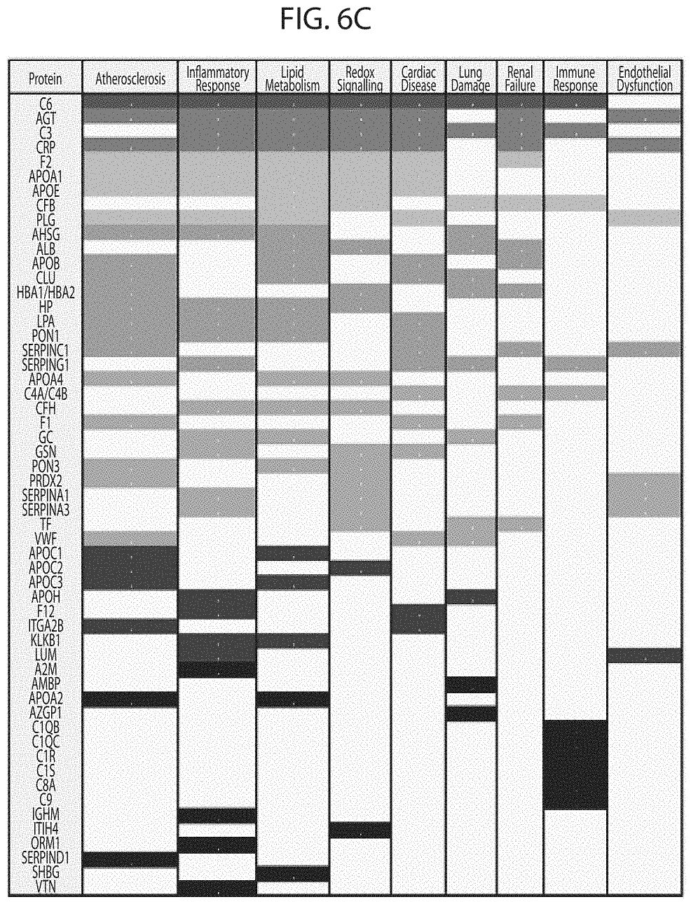

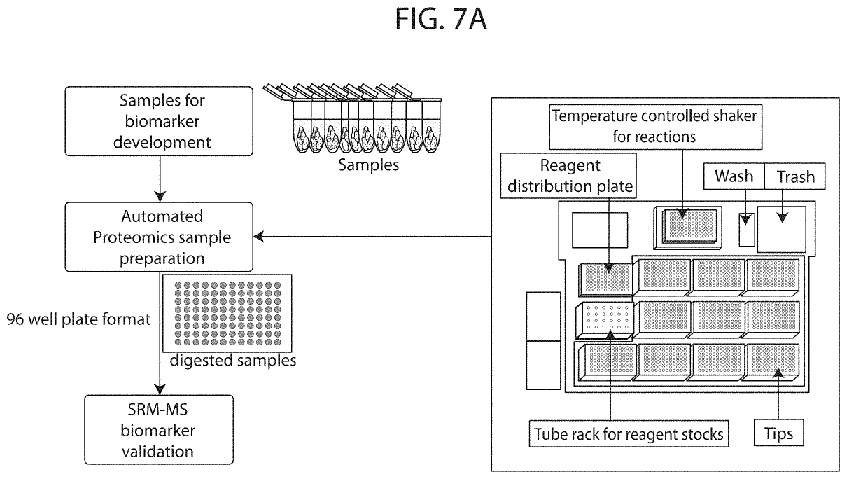

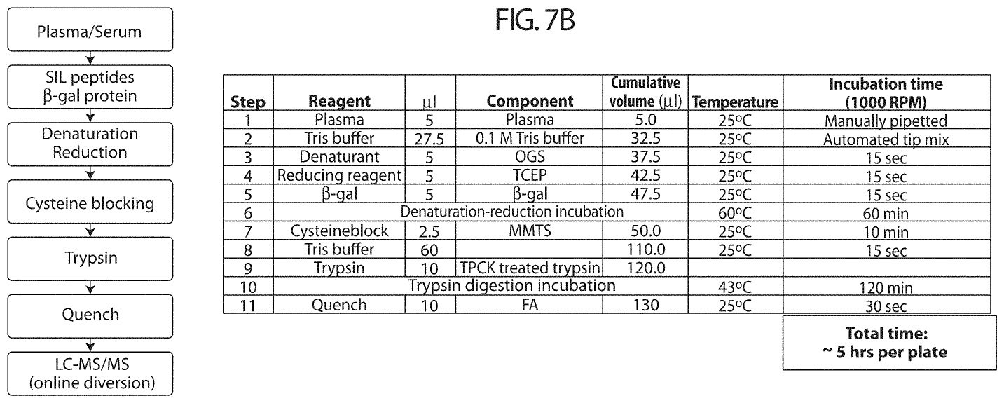

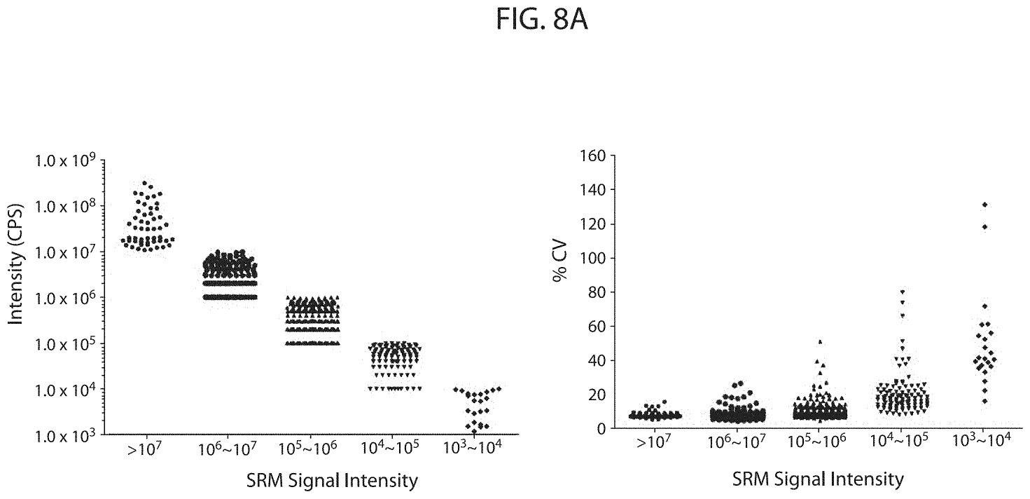

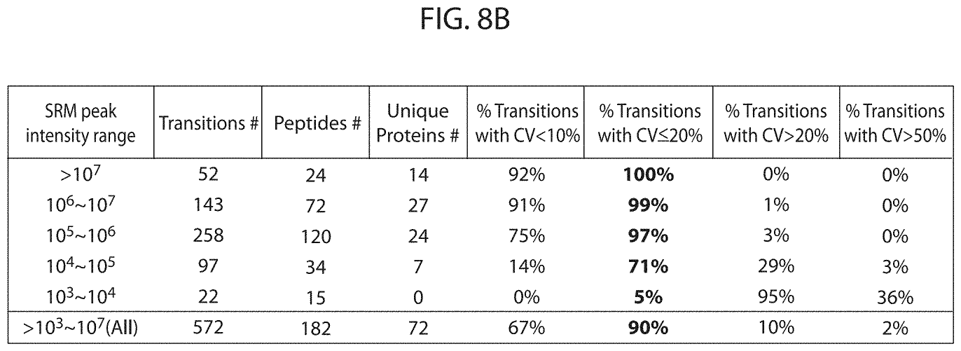

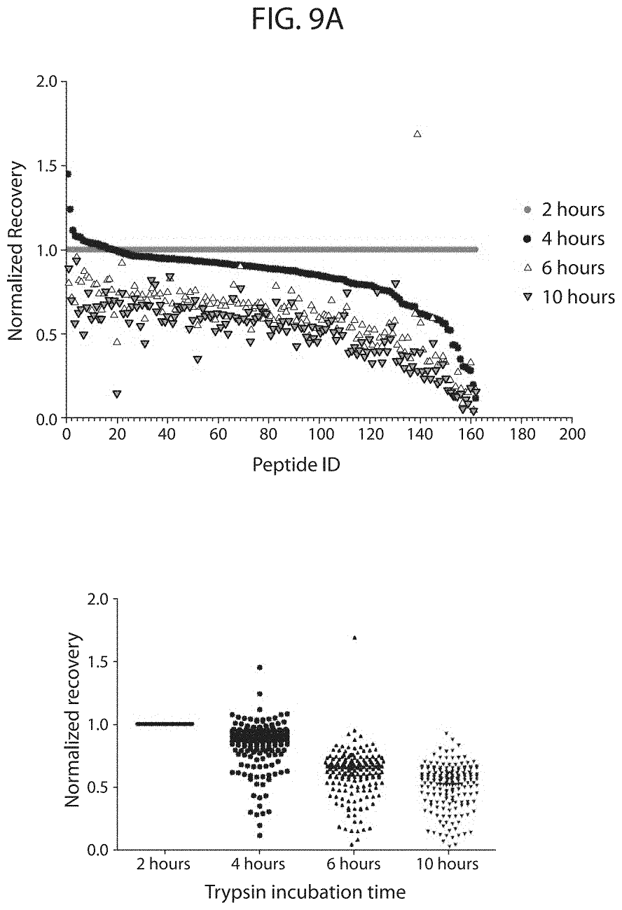

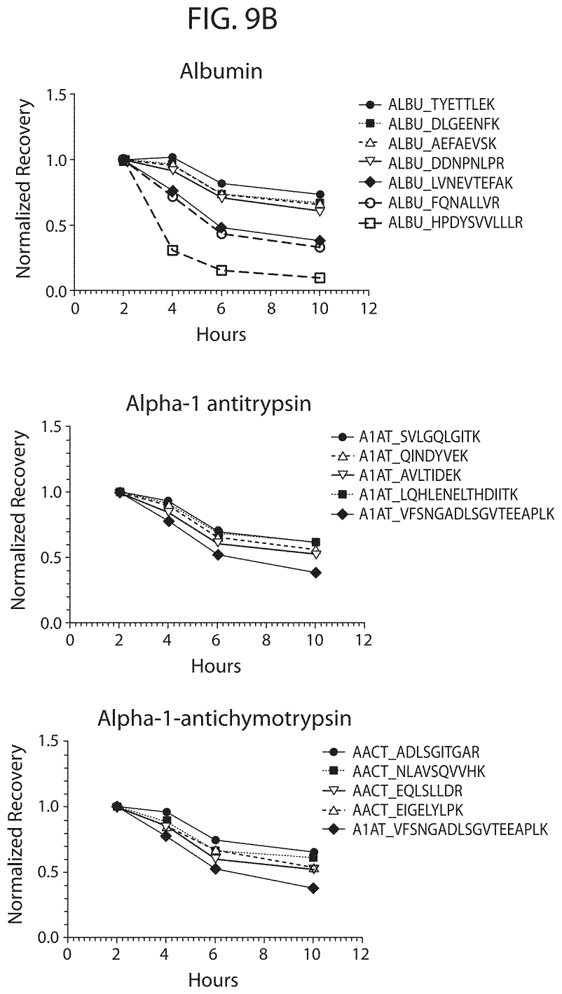

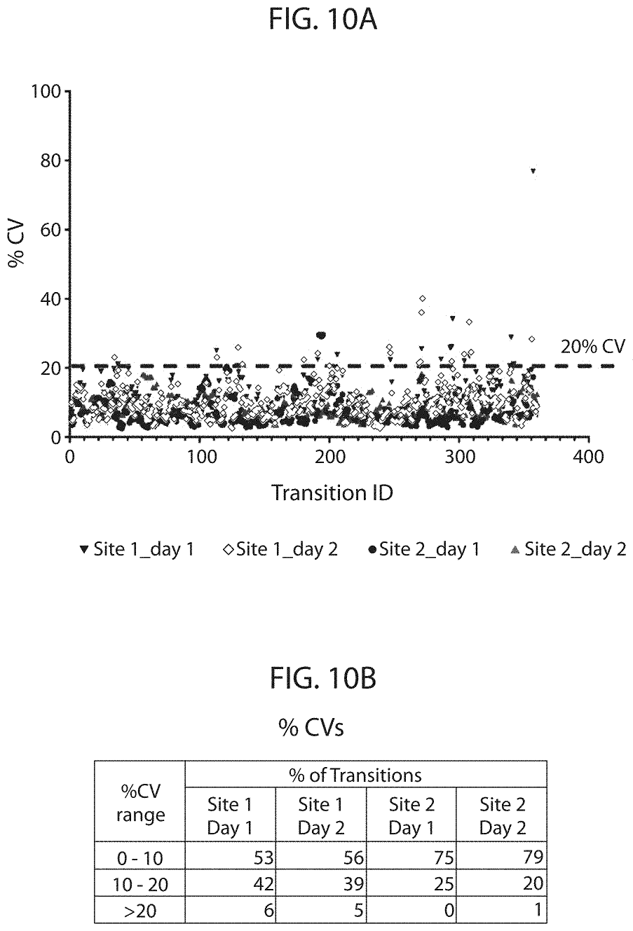

Data Independent Acquisition-Mass Spectrometry (DIA-MS). Next, we evaluated the applicability of our automated workflow for quantitative global biomarker discovery by DIA-MS^{18,19}. Digested plasma samples from healthy controls (n=8), and individuals with lower urinary tract pathology (n=8) were analyzed by SWATH® Acquisition on a TripleTOF® 6600 MS instrument, with three injections per sample. Overall, we observed 2205 peptides and 14262 ion transitions representing 668 proteins (FIG. 5B). The % CV of spiked β-gal in the samples prepared for SWATH analysis ranged from 6.2% to 13.5%, confirming reproducibility of the automated workflow (FIG. 5A). Furthermore, a comparison between two diseased and two control samples revealed high correlations between the quantified peptides and proteins (FIG. 5C). These correlations suggest that we can obtain quantitative information on thousands of peptides from hundreds of proteins by combining automated sample processing with data-independent MS²⁰.

There are many thousands of protease cleavage sites within the complex mixture of proteins in serum, plasma and other biological samples, and each protein has unique properties affecting cleavage site accessibility and the stability of the resulting peptides. It is therefore impossible to design a sample processing procedure that is optimal for every protein. The best alternative is to be as consistent as possible. The automated workflow presented here provides a solution for consistent enzymatic digestion. While the vast majority of peptides meet the best practice acceptance criterion for precision, selection of peptides/transitions with MS peak intensities above a certain threshold (such as 1×10⁴ cps for our IQ SRM panel implemented on a QTRAP 6500 MS) can drive CVs below 5% (as illustrated in FIG. 1). Combining the precision and reproducibility provided by automation with robust LC-SRM-MS methods will make it feasible to simultaneously investigate the validity of scores of candidate biomarkers across a large number of samples. Similarly, the uniformity and precision of our automated proteomics sample preparation workflow allows new technologies such as DIA MS to quantify many proteins simultaneously in a biomarker discovery study. In conclusion, application of our automated proteomics sample preparation in both targeted and global protein quantitation provides for improved reproducibility and throughput at all stages of the biomarker development pipeline.

In various embodiments, the automated sample preparation workflow presented herein increases throughput and vastly reduces the potential for errors resulting from inconsistent pipetting. When combined with online diversion for the final cleanup, the CV for the entire procedure was reduced to less than 20% for the vast majority (90%) of 572 transitions representing 182 peptides from 72 proteins.

Lengthy table referenced here
US20190369114A1-20191205-T00001
Please refer to the end of the specification for access instructions.

Lengthy table referenced here
US20190369114A1-20191205-T00002
Please refer to the end of the specification for access instructions.

Lengthy table referenced here
US20190369114A1-20191205-T00003
Please refer to the end of the specification for access instructions.

Lengthy table referenced here
US20190369114A1-20191205-T00004
Please refer to the end of the specification for access instructions.

Lengthy table referenced here
US20190369114A1-20191205-T00005
Please refer to the end of the specification for access instructions.

Lengthy table referenced here
US20190369114A1-20191205-T00006
Please refer to the end of the specification for access instructions.

Lengthy table referenced here
US20190369114A1-20191205-T00007
Please refer to the end of the specification for access instructions.

Lengthy table referenced here
US20190369114A1-20191205-T00008
Please refer to the end of the specification for access instructions.

Lengthy table referenced here
US20190369114A1-20191205-T00009
Please refer to the end of the specification for access instructions.

Lengthy table referenced here
US20190369114A1-20191205-T00010
Please refer to the end of the specification for access instructions.

Lengthy table referenced here
US20190369114A1-20191205-T00011
Please refer to the end of the specification for access instructions.

Lengthy table referenced here
US20190369114A1-20191205-T00012
Please refer to the end of the specification for access instructions.

Lengthy table referenced here
US20190369114A1-20191205-T00013
Please refer to the end of the specification for access instructions.

Lengthy table referenced here
US20190369114A1-20191205-T00014
Please refer to the end of the specification for access instructions.

Lengthy table referenced here
US20190369114A1-20191205-T00015
Please refer to the end of the specification for access instructions.

Lengthy table referenced here
US20190369114A1-20191205-T00016
Please refer to the end of the specification for access instructions.

Lengthy table referenced here
US20190369114A1-20191205-T00017
Please refer to the end of the specification for access instructions.

Lengthy table referenced here
US20190369114A1-20191205-T00018
Please refer to the end of the specification for access instructions.

The invention is further illustrated by the following examples which are intended to be purely exemplary of the invention, and which should not be construed as limiting the invention in any way. The following examples are illustrative only, and are not intended to limit, in any manner, any of the aspects described herein. The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### EXAMPLES

Reagents and Supplies:

Protein Preparation Kit containing denaturant (20% octyl-beta-glucopyranoside (OGS)), reducing reagent (50 mM Tris-(2-carboxyethyl)-phosphine (TCEP)), cysteine blocking reagent (200 mM methyl methane thiosulfate (MMTS)), and digestion buffer (0.1M Tris pH 8.3, 4 mM CaCl₂), and TPCK treated trypsin (Trypsin treated with L-(tosylamido-2-phenyl) ethyl chloromethyl ketone, 500 μg vials) were purchased from SCIEX. Tris base, CaCl₂, octyl-beta-glucopyranoside (OGS), and recombinant E. coli β-galactosidase (β-gal) were purchased from Sigma Aldrich. Tris(2-carboxyethyl)phosphine (TCEP), methyl methane thiosulfate (MMTS), acetonitrile (ACN) and formic acid (FA), 99.5+%, Optima LC/MS Grade were purchased from Thermo Fisher Scientific. 1 mL deep well plates were purchased from Beckman Coulter. Hardshell PCR plates were purchased from Bio-Rad. Xbridge Peptide BEH30 C18 2.1 mm×100 mm, 3.5 μm columns were purchased from Waters. Research grade ¹⁵N-labeled synthetic peptides were purchased from New England Peptide.

Pooled healthy human EDTA plasma was purchased from Bioreclamation. Pooled healthy human serum was purchased from Scantibodies Laboratory. Control remnant plasma samples from 24 individual ‘healthy’ patients were procured from the Cedars-Sinai Medical Center Core Laboratory. These specimens were deemed normal because every CBC parameter and comprehensive metabolic panel test were within normal ranges. Plasma samples from 48 post-menopausal women donors (mean age 62 years): 24 individuals without coronary artery disease (controls), and 24 individuals with known coronary artery disease (CAD) verified by quantitative coronary angiography as a component of ongoing collaborative research projects. These 48 “control” and “CAD” samples were used to aid in evaluating the influence of biological variability on the quality of workflow performance.

Multisite Testing.

Laboratory site 1 (Cedars Sinai Medical Center) used a high-flow LC-MS system as described above. Laboratory site 2 (SCIEX, Redwood City, Calif.) used a nanoLC 425 system (SCIEX) coupled to a QTRAP 6500 (SCIEX) with a microflow mode (5 μL/min) halopeptide (0.3×15 cm) LC column (Eksigent Technology).

### Example 1

Methods

Methods and any associated materials are available in online supplemental materials and methods.

Online Supplemental Materials and Methods:

Manual Protein Digestion:

Reagents were from the Protein Preparation Kit 100 Assay (SCIEX, Framingham, Mass.). The reaction was assembled with hand-held pipetters in a 1.5 mL microfuge tube. The reaction mix contained buffer, denaturant, reducing agent, β-gal (Sigma Aldrich, St Louis, Mo.), and SIL peptides (New England Peptide, Gardner, MA). 45 μL reaction mix was added to 5 μL healthy pooled plasma (Bioreclamation LLC, Hicksville, N.Y.). Proteins were denatured and reduced for 1 hour at 60° C. 2.5 alkylating reagent was added and the proteins were further incubated for 10 minutes at 25° C. After adding 60 μL buffer, 10 μL trypsin in 0.1% formic acid (FA) was added and the proteins were digested for 16-18 hours at 37° C. with a trypsin:substrate ratio of 1:20.

Solid Phase Extraction:

96-well Oasis HLB plates were used with a dedicated vacuum manifold (Waters Milford, Mass.). The HLB sorbent was wetted with methanol and then washed 3 times with 1 mL 0.1% formic acid. Each trypsin digest was acidified first with 300 μL of 0.1% FA and then with 400 μL of 4% phosphoric acid. The dilute acidified digests were slowly loaded (˜2 drops/s) onto the sorbent. The wells were washed 3 times with 1 mL 0.1% FA. Purified plasma peptides were eluted with 1 mL of 80% acetonitrile (ACN) in 0.1% FA, dried down using a SPD2010 SpeedVac system (Thermo Scientific, Waltham, Mass.), re-suspended in 2% ACN, and then transferred to autosampler vials for injection into the LC-MS system.

Digestion Optimization:

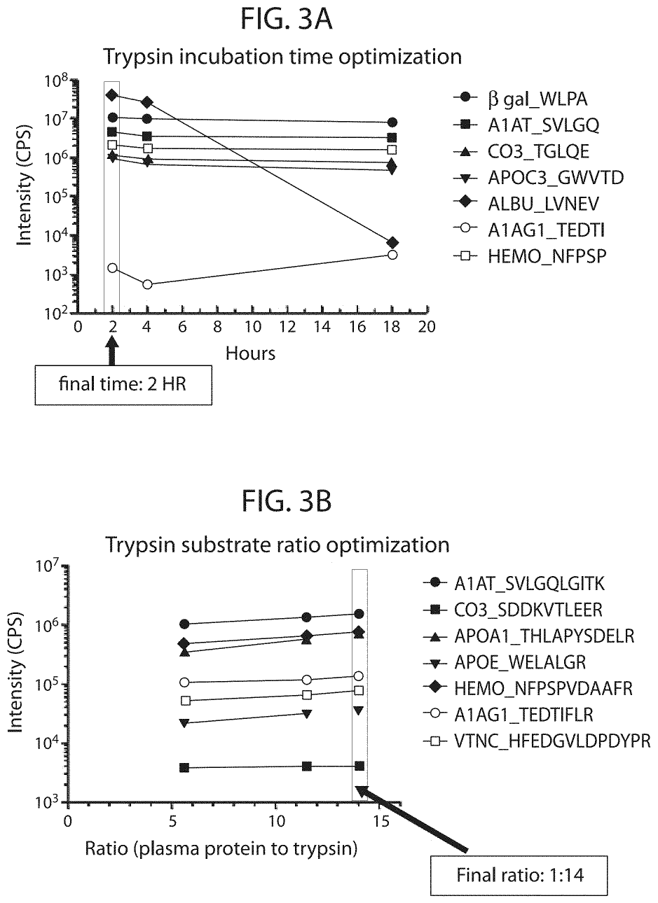

For optimization, samples were processed using automation (see “Automated Protein Digestion”) and the digestion plate was taken off-deck and sealed with a CAPMAT (Beckman Coulter, Indianapolis, Ind.) for two bench-top incubation steps: a) reduction for 1 hour at 60° C. and b) trypsin digestion at 37° C. while shaking at 1000 RPM. After these incubations, the plate was spun briefly and then returned to the Biomek for additional processing. The trypsin digestion conditions were adjusted in an effort to save time and simplify the procedure. After 2, 4 and 18 hours, we quenched digested plasma with formic acid and stored them at −80° C. until all time points were collected (FIG. 3A). Interestingly, the measured amount of albumin peptide LVNEVTEFAK (SEQ ID NO: 76) decreased after 18 hours, possibly due to protease activity in the plasma. Based on these results, the final method incorporated a 2-hour digestion. We next tested trypsin:substrate ratios of 1:5.6 (2 μL plasma), 1:11.5 (4 μL plasma) and 1:14 (5 μL plasma), assuming 70 μg protein per μl plasma (FIG. 3B). All three ratios yielded similar results, so we choose to use 5 μL of plasma as it allowed us to load more onto each LC-MS/MS run. For a two-hour digest of 5 μL plasma, the % CV for the complete process was 4.6% (set Gin Table 2b).

Online Desalting Optimization:

Online desalting was investigated as an alternative to SPE with the potential to reduce technical variation by eliminating the need to manually transfer samples and buffers to the SPE plate, control the vacuum pressure to adjust the flow rate, and dry and re-dissolve the eluted peptides. Two methods were tested. In simple online diversion, crude peptides were injected onto the C18 analytical column and the 2% organic flow-through was diverted to waste prior to gradient elution of peptides into the MS. Alternatively, a trap column was inserted upstream of the analytical column, and the trap column flow through was diverted to waste prior to gradient elution of peptides onto the analytical column. The advantage of the trap column is that it protects the more expensive analytical column from exposure to impurities. The advantage of the online diversion is that is a relative simpler solution. Replacing SPE with online desalting reduced the % CV from 26% to 9.1% for simple online diversion (set E in Table 3) or 9.4% for the trap column (set F, in Table 3). The final protocol used simple online diversion.

Automated Protein Digestion:

Samples were loaded into a deep well titer plate (Beckman Coulter) with single- or multi-channel pipettes (Eppendorf Hauppauge, N.Y.). The plate was then sealed with X-Pierce™ sealing film (Sigma Aldrich). All other liquid transfers were done on the Biomek NX^{P} Span-8 Laboratory Automation Workstation operated with Biomek software version 4.1 (Beckman Coulter). The instrument included a Shaking Peltier ALP (Inheco, Martinsried, Germany) with a deep well adapter for heating and mixing of samples. The plate was shaken at 1000 RPM for 15 seconds after each reagent addition. Protein Preparation Kit 100 Assay (SCIEX) was adapted for denaturation, reduction and cysteine blocking. For each reaction, the following were added sequentially to 5 μL plasma: 27.5 μL buffer (0.1M Tris pH 8.3, 4 mM CaCl₂), 5 μL Denaturant (20% N-octyl-glucoside, OGS), 5 μL internal standards (2 μg/μL β-galactosidase, Sigma Cat # G3153, 500 pmol/μL for both β-galactosidase and HSA SIL peptides), and 5 μL Reducing Reagent (50 mM TCEP). After all additions were made, the plate was shaken at 1000 RPM at 60° C. for 60 minutes. Next, 2.5 μL Cysteine Blocking Reagent (200 mM methyl methane-thiosulfonate (MMTS)) was added and the plate was shaken for 10 minutes at 1000 RPM. Finally, 10 μL of trypsin (2.5 μg/μL Trypsin (TPCK treated, SCIEX) in 0.1% FA) was added and the 96 well plate was incubated/shaken at 43° C. for 2 hours at 1000 RPM. After incubation, 10 μl of 10% FA was added to quench the reaction. The plate was spun for 5 minutes at 3400 RPM at 4° C. and 10 μL of the supernatant was transferred to 90 μL 2.2% ACN in 0.1% FA prior to SRM analysis (see bottom panel in FIG. 1A-FIG. 1B for protocol details).

SRM:

Tryptic plasma peptides and internal standards were analyzed on a Prominence UFLCXR HPLC system (Shimadzu, Japan) with a Waters Xbridge BEH30 C18 2.1 mm×100 mm, 3.5 μm column (Waters) flowing at 0.25 mL/min and 36° C. coupled to a QTRAP® 6500 or 5500 MS (SCIEX) with a Turbo V source. Analyst® software (version 1.6.2 for the QTRAP 6500 or 1.5.2 for the QTRAP 5500) was used to control the LC-MS/MS system and for data acquisition. Mobile phase A consisted of 2% ACN, 98% water, and 0.1% formic acid and mobile phase B of 95% ACN, 5% water, and 0.1% formic acid. The flow rate was 200 pt/min for QTRAP 5500 and 250 μL/min for QTRAP 6500. After loading, the column was equilibrated with 5% B for 5 minutes. Peptides were then eluted over 30 minutes with a linear 5% to 35% gradient of buffer B. The column was washed with 98% B for 10 minutes and then returned to 5% B for 5 minutes before loading the next sample. For online diversion, a two phase switching valve was used to divert the post-column eluent to waste before it entered the ion source. For trap diversion, a C18 trap column (Phenomenex, Torrance, Calif.) was inserted upstream of the Xbridge analytical column and the salt fraction was diverted to waste before entering the analytical column. The SRM MS parameters are shown in Table 5. Each sample was injected on the mass spectrometer in triplicate. All SRM data were processed using MultiQuant™ 2.1 Software (SCIEX).

### Example 2

Plasma from a Small Research Cohort of Lower Urinary Tract Pathology for SWATH-MS:

For investigation the utility of automated proteomics sample preparation workflow in SWATH-MS analysis, we selected 16 plasma samples from a lower urinary tract pathology study. We selected 8 controls and 8 individuals with lower urinary tract (diseased) pathology. Peripheral blood samples were obtained by sterile venipuncture into heparin sulfate-coated Vacutainer tubes (Becton Dickinson and Co. Franklin Lakes, N.J.). After centrifugation at 1500×g for 10 minutes, the plasma fraction was decanted off of the cellular pellet and aliquoted into cryotubes and stored at −80° C. until analysis.

SWATH-MS:

For the SWATH® acquisition experiment, we digested 5 μL plasma from 8 controls and 8 diseased plasma samples. The digestion was performed with the following modifications to the standard automated workflow: 1) cysteine residues were alkylated with iodoacetamide (with foil covering the digestion plate) for compatibility with our spectral library; 2) the trypsin digestion time was extended to 3 hours; and 3) the samples were manually desalted by SPE. We re-suspended the dried peptides at approximately 0.5 μg digested plasma proteins/μL in a solution of indexed retention time standard peptides (iRTs, Biognosys) in 0.1% FA. To confirm the reproducibility of sample preparation, we quantified β-gal by SRM in a 0.5 μg sample. For SWATH, we separated peptides from 2 μg plasma protein on a NanoLC™ 415 System (SCIEX) operating in trap-elute mode at microflow rates. A 0.3×150 cm ChromXP™ column (SCIEX) was used with a short gradient (3-35% solvent B in 60 min, 35-85% gradient for 3 min, hold at 85% for 5 min, re-equilibrate at 3% B for 7 min (Solvent B: 100% ACN, 0.1% FA in water) at 5 μL/min (total run time 75 min). The MS analysis was performed on a TripleTOF® 6600 system (SCIEX) using a DuoSpray™ Source with a 25 μm I.D. hybrid electrode (SCIEX). Variable window SWATH acquisition methods were built using Analyst® TF Software 1.7. A full MS1 scan (400-1250 Da) was acquired followed by 100 Q1 variable isolation windows across the mass range (400-1250 Da) for improved data quality through increased specificity. Variable sized Q1 windows optimized based on precursor density further increased specificity while ensuring broad mass range coverage (1 MS1 scan at 250 ms dwell followed by 100 VW SWATH MS/MS scans at 30 msec accumulation per window). Mass spectrometry parameters were: GS1=5, GS2=20, curtain gas=25, voltage=5500, temperature=100, rolling collision energy with collision energy spread (CES) of 5 per SWATH window, declustering potential=80.

Targeted Data Analysis for SWATH-MS:

SWATH-MS *.wiff files from the data-independent acquisition were first converted to profile mzML using ProteoWizard v.3.0.6002. The whole process of SWATH-targeted data analysis was carried out using OpenSWATH v.2.0.0 running on an internal computing cluster. OpenSWATH utilizes a target-decoy scoring system (PyProphet v.0.13.3) such as mProphet to estimate the identification of FDR. Based on the Twin Study Library, OpenSWATH first identified the peak groups from all individual SWATH maps at a global peptide FDR of 1% and aligned them between SWATH maps based on the clustering behaviors of retention time in each run with a non-linear alignment algorithm. For this analysis, the MS runs were realigned to each other using the LOcally WEighted Scatterplot Smoothing method and the peak group clustering was performed using the LocalMST method. Specifically, only those peptide peak groups that deviate within 3 standard deviations from the retention time were reported and considered for alignment with the max FDR quality of 5% (quality cutoff to still consider a feature for alignment). Next, to obtain high-quality quantitative data at the protein level, we discarded those proteins whose peptides were shared between multiple different proteins (non-proteotypic peptides). Quantitative peptide and protein level summary outputs were then used for all downstream biological analysis.

### Example 3

Quantifier Transition Validation.

Three to five transitions were measured for each peptide. The transition with the highest peak area was selected as the quantifier and other transitions were designated as qualifiers. Transition ratios were calculated by dividing the average peak area (n=2 digestions) of a qualifier by the quantifier.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

### REFERENCES

1. McDonald, W. H. & Yates, J. R., 3rd. Shotgun proteomics and biomarker discovery. Dis Markers 18, 99-105 (2002).
2. Anderson, L. & Hunter, C. L. Quantitative mass spectrometric multiple reaction monitoring assays for major plasma proteins. Molecular & Cellular Proteomics 5, 573-588 (2006).
3. Toga, A. W. et al. Big biomedical data as the key resource for discovery science. J Am Med Inform Assoc 22, 1126-1131, doi:10.1093/jamia/ocv077 (2015).
4. Domon, B. & Aebersold, R. Options and considerations when selecting a quantitative proteomics strategy. Nat Biotechnol 28, 710-721, doi:10.1038/nbt.1661 (2010).
5. Smith, R. D. Mass spectrometry in biomarker applications: from untargeted discovery to targeted verification, and implications for platform convergence and clinical application. Clin Chem 58, 528-530, doi:10.1373/clinchem.2011.180596 (2012).
6. Liu, Y. et al. Quantitative variability of 342 plasma proteins in a human twin population. Mol Syst Biol 11, 786, doi:10.15252/msb.20145728 (2015).
7. McIntosh, M. & Fitzgibbon, M. Biomarker validation by targeted mass spectrometry. Nat Biotechnol 27, 622-623, doi:10.1038/nbt0709-622 (2009).
8. Kennedy, J. J. et al. Demonstrating the feasibility of large-scale development of standardized assays to quantify human proteins. Nat Methods 11, 149-155, doi:10.1038/nmeth.2763 (2014).
9. Kruger, T., Lehmann, T. & Rhode, H. Effect of quality characteristics of single sample preparation steps in the precision and coverage of proteomic studies—a review. Anal Chim Acta 776, 1-10, doi:10.1016/j.aca.2013.01.020 (2013).
10. Dittrich, J., Becker, S., Hecht, M. & Ceglarek, U. Sample preparation strategies for targeted proteomics via proteotypic peptides in human blood using liquid chromatography tandem mass spectrometry. Proteomics Clin Appl 9, 5-16, doi:10.1002/prca.201400121 (2015).
11. Addona, T. A. et al. Multi-site assessment of the precision and reproducibility of multiple reaction monitoring-based measurements of proteins in plasma. Nat Biotechnol 27, 633-641, doi:10.1038/nbt.1546 (2009).
12. Lowenthal, M. S., Liang, Y., Phinney, K. W. & Stein, S. E. Quantitative bottom-up proteomics depends on digestion conditions. Anal Chem 86, 551-558, doi:10.1021/ac4027274 (2014).
13. van den Broek, I. et al. Automated Multiplex LC-MS/MS Assay for Quantifying Serum Apolipoproteins A-I, B, C-I, C-II, C-III, and E with Qualitative Apolipoprotein E Phenotyping. Clin Chem 62, 188-197, doi:10.1373/clinchem.2015.246702 (2016).
14. Zhang, Y. V. & Rockwood, A. Impact of automation on mass spectrometry. Clin Chim Acta 450, 298-303, doi:10.1016/j.cca.2015.08.027 (2015).
15. Grant, R. P. & Hoofnagle, A. N. From lost in translation to paradise found: enabling protein biomarker method transfer by mass spectrometry. Clin Chem 60, 941-944 (2014).
16. Hoofnagle, A. N. Quantitative clinical proteomics by liquid chromatography-tandem mass spectrometry: assessing the platform. Clin Chem 56, 161-164, doi:10.1373/clinchem.2009.134049 (2010).
17. Christie Hunter, Q. F., Mike Kowalski, Jennifer E. Van Eyk. Automating Protein Digestion for Reproducible Proteomics, Technote, SCIEX, 2014.
18. Sajic, T., Liu, Y. & Aebersold, R. Using data-independent, high-resolution mass spectrometry in protein biomarker research: perspectives and clinical applications. Proteomics Clin Appl 9, 307-321, doi:10.1002/prca.201400117 (2015).
19. Croft, N. P. et al. Simultaneous Quantification of Viral Antigen Expression Kinetics Using Data-Independent (DIA) Mass Spectrometry. Molecular & cellular proteomics: MCP 14, 1361-1372, doi:10.1074/mcp.M114.047373 (2015).
20. Shao, S., Guo, T. & Aebersold, R. Mass spectrometry-based proteomic quest for diabetes biomarkers. Biochim Biophys Acta 1854, 519-527, doi:10.1016/j.bbapap.2014.12.012 (2015).

LENGTHY TABLES
The patent application contains a lengthy table section. A copy of the table is available in electronic form from the USPTO web site (http://seqdata.uspto.gov/?pageRequest=docDetail&DocID=US20190369114A1). An electronic copy of the table will also be available from the USPTO upon request and payment of the fee set forth in 37 CFR 1.19(b)(3).

1. A protein panel for assessing and/or determining the state of health of a subject, the protein panel comprising one or more proteins listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integral alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

2. The protein panel of claim 1, wherein the one or more proteins are biomarkers for one or more functions or combination thereof, and
wherein the functions are inflammatory response, lipid metabolism, redox signaling, immune response, endothelial dysfunction, or coagulation.

3. (canceled)

4. (canceled)

5. The protein panel of claim 1, wherein the one or more proteins are biomarkers for one or more diseases or combination thereof.

6. The protein panel of claim 5, wherein the disease is cardiovascular disease.

7. The protein panel of claim 5, wherein the disease is cardiovascular disease and the proteins are
any one or more of Angiotensinogen (UniProt Accession No. P01019) (SEQ ID NO: 14), Apolipoprotein A-I (UniProt Accession No. P02647) (SEQ ID NO: 21), Apolipoprotein B-100 (UniProt Accession No. P04114) (SEQ ID NO: 43), Apolipoprotein E (UniProt Accession No. P02649) (SEQ ID NO: 22), Complement C3 (UniProt Accession No. P01024) (SEQ ID NO: 16), Complement C4-A (UniProt Accession No. P0C0L4) (SEQ ID NO: 59), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Gelsolin (UniProt Accession No. P06396) (SEQ ID NO: 51), Integrin alpha-Iib (UniProt Accession No. P08514) (SEQ ID NO: 54), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Antithrombin-III (UniProt Accession No. P01008) (SEQ ID NO: 11), Plasma protease C1 inhibitor (UniProt Accession No. P05155) (SEQ ID NO: 48), von Willebrand factor (UniProt Accession No. P04275) (SEQ ID NO: 45), or combinations thereof, or
any one or more of Angiotensinogen (UniProt Accession No. P01019) (SEQ ID NO: 14), Apolipoprotein A-I (UniProt Accession No. P02647) ((SEQ ID NO: 21), Apolipoprotein B-100 (UniProt Accession No. P04114) (SEQ ID NO: 43), Apolipoprotein E (UniProt Accession No. P02649) (SEQ ID NO: 22), Complement C3 (UniProt Accession No. P01024) (SEQ ID NO: 16), Complement C4-A (UniProt Accession No. P0C0L4) (SEQ ID NO: 59), Complement C4-B (UniProt Accession No. P0C0L5) (SEQ ID NO: 60), Complement C5 (UniProt Accession No. P01031) (SEQ ID NO: 17), Clusterin (UniProt Accession No. P10909) (SEQ ID NO: 61), C-reactive protein (UniProt Accession No. P02741) (SEQ ID NO: 28), Coagulation factor X (UniProt Accession No. P00742) (SEQ ID NO: 7), Coagulation factor XII (UniProt Accession No. P00748) (SEQ ID NO: 9), Prothrombin (UniProt Accession No. P00734) (SEQ ID NO: 4), Gelsolin (UniProt Accession No. P06396) (SEQ ID NO: 51), Integrin alpha-Iib (UniProt Accession No. P08514) (SEQ ID NO: 54), Apolipoprotein(a) (UniProt Accession No. P08519) (SEQ ID NO: 55), Plasminogen (UniProt Accession No. P00747) (SEQ ID NO: 8), Serum paraoxonase/arylesterase 1 (UniProt Accession No. P27169) (SEQ ID NO: 65), Antithrombin-III (UniProt Accession No. P01008) (SEQ ID NO: 11), Alpha-2-antiplasmin (UniProt Accession No. P08697) (SEQ ID NO: 57), Plasma protease C1 inhibitor (UniProt Accession No. P05155) (SEQ ID NO: 48), von Willebrand factor (UniProt Accession No. P04275) (SEQ ID NO: 45), or combinations thereof.

8. (canceled)

9. (canceled)

10. A method for assessing and/or determining the state of health of a subject, the method comprising:
obtaining a sample from a subject,
contacting the sample with one or more proteases so as to obtain a digested sample, wherein the digested sample comprises one or more peptides;
analyzing the digested sample by mass spectrometry so as to obtain mass spectrometry data;
correlating the mass spectrometry data to the one or more peptides; and
correlating the one or more peptides to one or more proteins, wherein the one or more proteins are listed in the following Table:

| | UniProt | SEQ |
|---|---|---|
| Protein Name | Accession No. | ID NO: |
| Apolipoprotein L1 | O14791 | 1 |
| Ceruloplasmin | P00450 | 2 |
| Beta-galactosidase | P00722 | 3 |
| Prothrombin | P00734 | 4 |
| Complement C1r subcomponent | P00736 | 5 |
| Haptoglobin | P00738 | 6 |
| Coagulation factor X | P00742 | 7 |
| Plasminogen | P00747 | 8 |
| Coagulation factor XII | P00748 | 9 |
| Complement factor B | P00751 | 10 |
| Antithrombin-III | P01008 | 11 |
| Alpha-1-antitrypsin | P01009 | 12 |
| Alpha-1-antichymotrypsin | P01011 | 13 |
| Angiotensinogen | P01019 | 14 |
| Alpha-2-macroglobulin | P01023 | 15 |
| Complement C3 | P01024 | 16 |
| Complement C5 | P01031 | 17 |
| Ig gamma-3 chain C region | P01860 | 18 |
| Ig mu chain C region | P01871 | 19 |
| Ig alpha-1 chain C region | P01876 | 20 |
| Apolipoprotein A-I | P02647 | 21 |
| Apolipoprotein E | P02649 | 22 |
| Apolipoprotein A-II | P02652 | 23 |
| Apolipoprotein C-I | P02654 | 24 |
| Apolipoprotein C-II | P02655 | 25 |
| Apolipoprotein C-III | P02656 | 26 |
| Fibrinogen | P02671 | 27 |
| C-reactive protein | P02741 | 28 |
| Complement C1q subcomponent subunit B | P02746 | 29 |
| Complement C1q subcomponent subunit C | P02747 | 30 |
| Complement component C9 | P02748 | 31 |
| Leucine-rich alpha-2-glycoprotein | P02750 | 32 |
| Protein AMBP | P02760 | 33 |
| Alpha-1-acid glycoprotein 1 | P02763 | 34 |
| Alpha-2-HS-glycoprotein | P02765 | 35 |
| Serum albumin | P02768 | 36 |
| Vitamin D-binding protein | P02774 | 37 |
| Serotransferrin | P02787 | 38 |
| Hemopexin | P02790 | 39 |
| Plasma kallikrein | P03952 | 40 |
| C4b-binding protein alpha chain | P04003 | 41 |
| Vitronectin | P04004 | 42 |
| Apolipoprotein B-100 | P04114 | 43 |
| Alpha-1B-glycoprotein | P04217 | 44 |
| von Willebrand factor | P04275 | 45 |
| Sex hormone-binding globulin | P04278 | 46 |
| Plasma serine protease inhibitor | P05154 | 47 |
| Plasma protease C1 inhibitor | P05155 | 48 |
| Thyroxine-binding globulin | P05543 | 49 |
| Heparin cofactor 2 | P05546 | 50 |
| Gelsolin | P06396 | 51 |
| Apolipoprotein A-IV | P06727 | 52 |
| Complement component C8 alpha chain | P07357 | 53 |
| Integral alpha-Iib | P08514 | 54 |
| Apolipoprotein(a) | P08519 | 55 |
| Complement factor H | P08603 | 56 |
| Alpha-2-antiplasmin | P08697 | 57 |
| Complement C1s subcomponent | P09871 | 58 |
| Complement C4-A | P0C0L4 | 59 |
| Complement C4-B | P0C0L5 | 60 |
| Clusterin | P10909 | 61 |
| Inter-alpha-trypsin inhibitor heavy chain H2 | P19823 | 62 |
| Inter-alpha-trypsin inhibitor heavy chain H1 | P19827 | 63 |
| Zinc-alpha-2-glycoprotein | P25311 | 64 |
| Serum paraoxonase/arylesterase 1 | P27169 | 65 |
| Peroxiredoxin-2 | P32119 | 66 |
| Serum amyloid A-4 protein | P35542 | 67 |
| Afamin | P43652 | 68 |
| Lumican | P51884 | 69 |
| Hemoglobin subunit alpha | P69905 | 70 |
| Inter-alpha-trypsin inhibitor heavy chain H4 | Q14624 | 71 |
| Serum paraoxonase/lactonase 3 | Q15166 | 72 |

and; comparing the presence or level of the one or more proteins in the sample from the subject to the presence or level of the one or more proteins in a reference sample so as to assess and/or determine the state of health of the subject.

11. (canceled)

12. (canceled)

13. (canceled)

14. (canceled)

15. (canceled)

16. The method of claim 10, wherein the mass spectrometry is selected reaction monitoring (SRM) mass spectrometry or multiple reaction monitoring (MRM) mass spectrometry.

17. The method of claim 16, wherein the one or more peptides are correlated to the one or more proteins according to Table 5, or
wherein the mass spectrometry data comprises one or more Q1/Q3 mass value pairs, wherein the Q1/Q3 mass value pairs are correlated to the one or more peptides, and the one or more peptides are correlated to the one or more proteins according to Table 5.

18. (canceled)

19. The method of claim 16, wherein the one or more peptides are correlated to the one or more proteins according to Table 7, or
wherein the mass spectrometry data comprises one or more precursor ions and one or more product ions, wherein the precursor ions and the product ions are correlated to the one or more peptides, and the one or more peptides are correlated to the one or more proteins according to Table 7.

20. (canceled)

21. The method of claim 16, wherein the one or more peptides are correlated to the one or more proteins according to Table 8, or
wherein the mass spectrometry data comprises one or more precursor ions and one or more product ions, wherein the precursor ions and the product ions are correlated to the one or more peptides, and the one or more peptides are correlated to the one or more proteins according to Table 8.

22. (canceled)

23. The method of claim 10, wherein the mass spectrometry is parallel reaction monitoring (PRM) mass spectrometry.

24. The method of claim 23, wherein the one or more peptides are correlated to the one or more proteins according to Table 9, or wherein the mass spectrometry data comprises one or more precursor ions and one or more product ions, wherein the precursor ions and the product ions are correlated to the one or more peptides, and the one or more peptides are correlated to the one or more proteins according to Table 9.

25. (canceled)

26. The method of claim 23, wherein the one or more peptides are correlated to the one or more proteins according to Table 10, or
wherein the mass spectrometry data comprises one or more precursor ions and one or more product ions, wherein the precursor ions and the product ions are correlated to the one or more peptides, and the one or more peptides are correlated to the one or more proteins according to Table 10.

27. (canceled)

28. The method of claim 10, wherein the mass spectrometry is data dependent acquisition (DDA) mass spectrometry.

29. The method of claim 28, wherein the one or more peptides are correlated to the one or more proteins according to Table 11, or wherein the mass spectrometry data comprises one or more precursor ions, wherein the precursor ions are correlated to the one or more peptides, and the one or more peptides are correlated to the one or more proteins according to Table 11.

30. (canceled)

31. The method of claim 28, wherein the one or more peptides are correlated to the one or more proteins according to Table 12, or wherein the mass spectrometry data comprises one or more precursor ions and one or more product ions, wherein the precursor ions and the product ions are correlated to the one or more peptides, and the one or more peptides are correlated to the one or more proteins according to Table 12.

32. (canceled)

33. The method of claim 10, wherein the mass spectrometry is data independent acquisition (DIA) mass spectrometry.

34. The method of claim 33, wherein the one or more peptides are correlated to the one or more proteins according to Table 13, or wherein the mass spectrometry data comprises one or more precursor ions and one or more product ions, wherein the precursor ions and the product ions are correlated to the one or more peptides, and the one or more peptides are correlated to the one or more proteins according to Table 13.

35. (canceled)

36. The method of claim 33, wherein the one or more peptides are correlated to the one or more proteins according to Table 14, or wherein the mass spectrometry data comprises one or more precursor ions and one or more product ions, wherein the precursor ions and the product ions are correlated to the one or more peptides, and the one or more peptides are correlated to the one or more proteins according to Table 14.

37. (canceled)
